(19) United States
(12) Patent Application Publication
(10) Pub. No.: US 2009/0233928 A1
(43) Pub. Date: Sep. 17, 2009

(54) IMIDAZOLO-5-YL-2-ANILO-PYRIMIDINES AS AGENTS FOR THE INHIBITION OF CELL PROLIFERATION

(76) Inventors: David Andrews, Cheshire (GB); Maurice Raymond Finlay, Cheshire (GB); Clive Green, Cheshire (GB); Clifford Jones, Cheshire (GB); Vibha Oza, Acton, MA (US)

Correspondence Address: MORGAN LEWIS & BOCKIUS LLP, 1111 PENNSYLVANIA AVENUE NW, WASHINGTON, DC, 20004, US

(21) Appl. No.: 11/817,389

(22) PCT Filed: Mar. 7, 2006
(86) PCT No.: PCT/GB2006/000813

§ 371 Date: Nov. 25, 2008

(30) Foreign Application Priority Data
Mar. 8, 2005 (GB) 0504753.5

(51) Int. Cl. A61K 31/5377; C07D 401/14; A61K 31/506; C07D 413/14

(52) U.S. Cl. 514/235.8; 544/297; 514/275; 544/122

(57) ABSTRACT

Compounds of the formula (I):

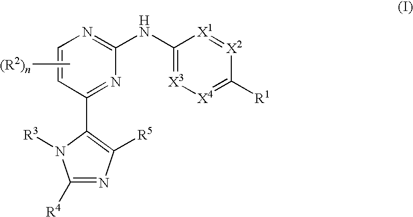

wherein variable groups are as defined within and a pharmaceutically acceptable salts and in vivo hydrolysable esters are described. Also described are processes for their preparation and their use as medicaments, particularly medicaments for producing a cell cycle inhibitory (anti-cell-proliferation) effect in a warm-blooded animal, such as man.

The invention relates to pyrimidine derivatives, or pharmaceutically acceptable salts or in vivo hydrolysable esters thereof, which possess cell-cycle inhibitory activity and are accordingly useful for their anti-cell-proliferation (such as anti-cancer) activity and are therefore useful in methods of treatment of the human or animal body. The invention also relates to processes for the manufacture of said pyrimidine derivatives, to pharmaceutical compositions containing them and to their use in the manufacture of medicaments of use in the production of an anti-cell-proliferation effect in a warm-blooded animal such as man.

The cell cycle is fundamental to the survival, regulation and proliferation of cells and is highly regulated to ensure that each step progresses in a timely and orderly manner. The progression of cells through the cell cycle arises from the sequential activation and de-activation of several members of the cyclin-dependent kinase (CDK) family. The activation of CDKs is dependent on their interaction with a family of intracellular proteins called cyclins. Cyclins bind to CDKs and this association is essential for CDK activity (such as CDK1, CDK2, CDK4 and/or CDK6) within the cell. Different cyclins are expressed and degraded at different points in the cell cycle to ensure that activation and inactivation of CDKs occurs in the correct order for progression through the cell cycle.

Moreover, CDKs appear to be downstream of a number of oncogene signalling pathways. Deregulation of CDK activity by upregulation of cyclins and/or deletion of endogenous inhibitors appears to be an important axis between mitogenic signalling pathways and proliferation of tumour cells.

Accordingly it has been recognised that an inhibitor of cell cycle kinases, particularly inhibitors of CDK1, CDK2 and/or CDK4 (which operate at the G2/M, G1/S-S-G2/M and G1-S phases respectively) should be of value as an active inhibitor of cell proliferation, such as growth of mammalian cancer cells.

The inhibition of cell cycle kinases is expected to be of value in the treatment of disease states associated with aberrant cell cycles and cell proliferation such as cancers (solid tumours and leukemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

WO 02/20512, WO 03/076435, WO 03/076436, WO 03/076434, WO 03/076433 and WO 04/101549 describe certain 2-anilino-4-imidazolylpyrimidine derivatives that inhibit the effect of cell cycle kinases. The present invention is based on the discovery that a novel group of pyrimidines inhibit the effects of cell cycle kinases showing activity against CDK1, CDK2 and CDK4, particularly CDK2 and CDK4, and thus possess anti-cell-proliferation properties. We have surprisingly found that these compounds possess beneficial properties in terms of one or more of their pharmacological activity (particularly as compounds which inhibit the before mentioned CDKs) and/or pharmacokinetic, efficacious, metabolic and toxicological profiles that make them particularly suitable for in vivo administration to a warm blooded animal, such as man. In particular these compounds have very high levels of cell and enzyme potency and high levels of exposure in vivo.

Accordingly, the present invention provides a compound of formula (I):

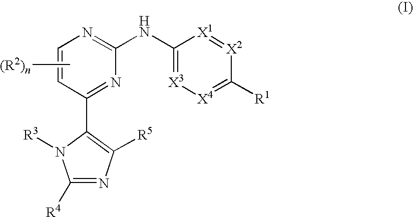

wherein:

R¹ is sulphamoyl, carbamoyl, a group -R⁶-R⁷ or a nitrogen linked 4-7 membered saturated ring which optionally contains an additional nitrogen, oxygen or sulphur atom; wherein said ring may be optionally substituted on carbon by one or more R⁸; and wherein if said ring contains an additional nitrogen atom that nitrogen may be optionally substituted by R⁹;

one of X¹, X², X³ and X⁴ is selected from ═N—, the other three X¹, X², X³ or X⁴ are independently selected from ═N— or ═C(R¹⁰)—;

R² is halo, nitro, cyano, hydroxy, trifluoromethyl, trifluoromethoxy, amino, carboxy, carbamoyl, mercapto, sulphamoyl, C₁₋₃alkyl, C₂₋₃alkenyl, C₂₋₃alkynyl, C₁₋₃alkoxy, C₁₋₃alkanoyl, N—(C₁₋₃alkyl)amino, N,N—(C₁₋₃alkyl)₂amino, C₁₋₃alkanoylamino, N—(C₁₋₃alkyl)carbamoyl, N,N—(C₁₋₃alkyl)₂carbamoyl, C₁₋₃alkylS(O)ₐ wherein a is 0 to 2, N—(C₁₋₃alkyl)sulphamoyl or N,N—(C₁₋₃alkyl)₂sulphamoyl; wherein R² may be optionally substituted on carbon by one or more R¹¹;

n is 0 to 2, wherein the values of R² may be the same or different;

R³ is hydrogen, C₁₋₆alkyl, C₂₋₆alkenyl, C₂₋₆alkynyl, carbocyclyl or heterocyclyl; wherein R³ may be optionally substituted on carbon by one or more R¹²; and wherein if said heterocyclyl contains an —NH— moiety that nitrogen may be optionally substituted by a group selected from R¹³;

R⁴, R⁵ and R⁸ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, C₃₋₈cycloalkyl or a 4-7 membered saturated heterocyclic group; wherein R⁴, R⁵ and R⁸ independently of each other may be optionally substituted on carbon by one or more R¹⁴; and wherein if said 4-7 membered saturated heterocyclic group contains an —NH— moiety that nitrogen may be optionally substituted by a group selected from R¹⁵;

R⁶ is —O—, —C(O)—, —N(R¹⁶)C(O)—, —C(O)N(R¹⁷)—, —S(O)ᵣ—, —OC(O)N(R¹⁸)SO₂—, —SO₂N(R¹⁹)— or —N(R²⁰)SO₂—; wherein R¹⁶, R¹⁷, R¹⁸, R¹⁹ and R²⁰ are independently hydrogen or C₁₋₆alkyl optionally substituted by one or more R²¹ and r is 0-2;

R⁷ is selected from C₁₋₁₆alkyl, C₂₋₆alkenyl, C₂₋₆alkynyl, carbocyclyl or heterocyclyl; wherein R⁷ may be optionally substituted on carbon by one or more R²²; and wherein if said heterocyclyl contains an —NH— moiety that nitrogen may be optionally substituted by a group selected from R²³;

R¹⁰ is selected from hydrogen, halo, nitro, cyano, hydroxy, amino, carboxy, carbamoyl, mercapto, C₁₋₆alkyl, C₁₋₆alkoxy, C₂₋₆alkenyl or C₂₋₆alkynyl;

R¹², R²¹ and R²² are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkoxyC₁₋₆alkoxy, C₁₋₆alkoxyC₁₋₆alkoxyC₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₁₋₆alkyl-R²⁴—, heterocyclylC₁₋₆alkyl-R²⁵—, carbocyclyl-R²⁶— or heterocyclyl-R²⁷—; wherein R¹², R²¹ and R²² independently of each other may be optionally substituted on carbon by one or more R²⁸; and wherein if said heterocyclyl contains an —NH— moiety that nitrogen may be optionally substituted by a group selected from R²⁹;

R²⁴, R²⁵, R²⁶ and R²⁷ are independently selected from —O—, —N(R³⁰)—, —C(O)—, —N(R³¹)C(O)—, —C(O)N(R³²)—, —S(O)ₛ—, —SO₂N(R³³)— or —N(R³⁴)SO₂—; wherein R³⁰, R³¹, R³², R³³ and R³⁴ are independently selected from hydrogen or C₁₋₆alkyl and s is 0-2;

R⁹, R¹³, R¹⁵, R²³ and R²⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; wherein R⁹, R¹³, R¹⁵, R²³ and R²⁹ independently of each other may be optionally substituted on carbon by one or more R³⁵; and

R¹¹, R¹⁴, R³⁵ and R²⁸ are independently selected from halo, nitro, cyano, hydroxy, trifluoromethoxy, trifluoromethyl, amino, carboxy, carbamoyl, mercapto, sulphamoyl, methyl, ethyl, cyclopropyl, cyclobutyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt or an in vivo hydrolysable ester thereof.

According to a further aspect of the present invention there is provided a compound of formula (I) wherein:

R¹ is sulphamoyl, carbamoyl, a group -R⁶-R⁷ or a nitrogen linked 4-7 membered saturated ring which optionally contains an additional nitrogen, oxygen or sulphur atom; wherein said ring may be optionally substituted on carbon by one or more R⁸; and wherein if said ring contains an additional nitrogen atom that nitrogen may be optionally substituted by R⁹;

one of X¹, X², X³ and X⁴ is selected from ═N—, the other three X¹, X², X³ or X⁴ are independently selected from ═N— or ═C(R¹⁰)—;

R² is halo, nitro, cyano, hydroxy, trifluoromethyl, trifluoromethoxy, amino, carboxy, carbamoyl, mercapto, sulphamoyl, C₁₋₃alkyl, C₂₋₃alkenyl, C₂₋₃alkynyl, C₁₋₃alkoxy, C₁₋₃alkanoyl, N—(C₁₋₃alkyl)amino, N,N—(C₁₋₃alkyl)₂amino, C₁₋₃alkanoylamino, N—(C₁₋₃alkyl)carbamoyl, N,N—(C₁₋₃alkyl)₂carbamoyl, C₁₋₃alkylS(O)ₐ wherein a is 0 to 2, N—(C₁₋₃alkyl)sulphamoyl or N,N—(C₁₋₃alkyl)₂sulphamoyl; wherein R² may be optionally substituted on carbon by one or more R¹¹;

n is 0 to 2, wherein the values of R² may be the same or different;

R³ is hydrogen, C₁₋₆alkyl, C₂₋₆alkenyl, C₂₋₆alkynyl, carbocyclyl or heterocyclyl; wherein R³ may be optionally substituted on carbon by one or more R¹²; and wherein if said heterocyclyl contains an —NH— moiety that nitrogen may be optionally substituted by a group selected from R¹³;

R⁴, R⁵ and R⁸ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, C₃₋₈cycloalkyl or a 4-7 membered saturated heterocyclic group; wherein R⁴, R⁵ and R⁸ independently of each other may be optionally substituted on carbon by one or more R¹⁴; and wherein if said 4-7 membered saturated heterocyclic group contains an —NH— moiety that nitrogen may be optionally substituted by a group selected from R¹⁵;

R⁶ is —C(O)—, —N(R¹⁶)C(O)—, —C(O)N(R¹⁷)—, —S(O)ᵣ—, —OC(O)N(R¹⁸)SO₂—, —SO₂N(R¹⁹)— or —N(R²⁰)SO₂—; wherein R¹⁶, R¹⁷, R¹⁸, R¹⁹ and R²⁰ are independently hydrogen or C₁₋₆alkyl optionally substituted by one or more R²¹ and r is 0-2;

R⁷ is selected from C₁₋₆alkyl, C₂₋₆alkenyl, C₂₋₆alkynyl, carbocyclyl or heterocyclyl; wherein R⁷ may be optionally substituted on carbon by one or more R²²; and wherein if said heterocyclyl contains an —NH— moiety that nitrogen may be optionally substituted by a group selected from R²³;

R¹⁰ is selected from hydrogen, halo, nitro, cyano, hydroxy, amino, carboxy, carbamoyl, mercapto, C₁₋₆alkyl, C₁₋₆alkoxy, C₂₋₆alkenyl or C₂₋₆alkynyl;

R¹², R²¹ and R²² are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkoxyC₁₋₆alkoxy, C₁₋₆alkoxyC₁₋₆alkoxyC₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₁₋₆alkyl-R²⁴—, heterocyclylC₁₋₆alkyl-R²⁵—, carbocyclyl-R²⁶— or heterocyclyl-R²⁷—; wherein R¹², R²¹ and R²² independently of each other may be optionally substituted on carbon by one or more R²⁸; and wherein if said heterocyclyl contains an —NH— moiety that nitrogen may be optionally substituted by a group selected from R²⁹;

R²⁴, R²⁵, R²⁶ and R²⁷ are independently selected from —O—, —N(R³⁰)—, —C(O)—, —N(R³W)C(O)—, —C(O)N(R³²)—, —S(O)—, —SO₂N(R³³)— or —N(R³⁴)SO₂—; wherein R³⁰, R³¹, R³², R³³ and R³⁴ are independently selected from hydrogen or C₁₋₆alkyl and s is 0-2;

R⁹, R¹³, R¹⁵, R²³ and R²⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; wherein R⁹, R¹³, R¹⁵, R²³ and R²⁹ independently of each other may be optionally substituted on carbon by one or more R³⁵; and

R¹¹, R¹⁴, R³⁵ and R²⁸ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt or an in vivo hydrolysable ester thereof.

In this specification the term “alkyl” includes both straight and branched chain alkyl groups but references to individual alkyl groups such as “propyl” are specific for the straight chain version only. For example, “C₁₋₆alkyl” and “C₁₋₄alkyl” include methyl, ethyl, propyl, isopropyl and t-butyl. However, references to individual alkyl groups such as ‘propyl’ are specific for the straight chained version only and references to individual branched chain alkyl groups such as ‘isopropyl’ are specific for the branched chain version only. A similar convention applies to other radicals, for example “carbocyclylC₁₋₆alkyl-R¹⁸” includes carbocyclylmethyl-R¹⁸, 1-carbocyclylethyl-R¹⁸ and 2-carbocyclylethyl-R¹⁸. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “4-7 membered saturated heterocyclic group” is a saturated monocyclic ring containing 4-7 atoms of which at least one atom is chosen from nitrogen, sulphur or oxygen, which may, unless otherwise specified, be carbon or nitrogen linked, wherein a —CH₂— group can optionally be replaced by a —C(O)— and a sulphur atom may be optionally oxidised to form the S-oxides. Examples and suitable values of the term “4-7 membered saturated heterocyclic group” are morpholino, piperidyl, 1,4-dioxanyl, 1,3-dioxolanyl, 1,2-oxathiolanyl, imidazolidinyl, pyrazolidinyl, piperazinyl, thiazolidinyl, pyrrolidinyl, thiomorpholino, homopiperazinyl and tetrahydropyranyl.

A “nitrogen linked 4-7 membered saturated ring which optionally contains an additional nitrogen, oxygen or sulphur atom” is a saturated monocyclic ring containing 4-7 atoms linked to the X¹-X⁴ containing ring of formula (I) via a nitrogen atom contained in the ring. The ring optionally contains an additional heteroatom selected from nitrogen, sulphur or oxygen, wherein a —CH₂— group can optionally be replaced by a —C(O)—, and the optional sulphur atom may be optionally oxidised to form the S-oxides. Particular examples of a “nitrogen linked 4-7 membered saturated ring which optionally contains an additional nitrogen, oxygen or sulphur atom” are piperazin-1-yl and morpholino, particularly morpholino.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂-group can optionally be replaced by a —C(O)—, a ring nitrogen atom may optionally bear a C₁₋₆alkyl group and form a quaternary compound or a ring nitrogen and/or sulphur atom may be optionally oxidised to form the N-oxide and or the S-oxides. Examples and suitable values of the term “heterocyclyl” are morpholino, piperidyl, pyridyl, pyranyl, pyrrolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. In one aspect of the invention a “heterocyclyl” is a saturated, partially saturated or unsaturated, mono or bicyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl.

An example of “C₁₋₆alkanoyloxy” is acetoxy. Examples of “C₁₋₆alkoxycarbonyl” include methoxycarbonyl, ethoxycarbonyl, n- and t-butoxycarbonyl. Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “N—(C₁₋₆alkyl)amino” include methylamino and ethylamino. Examples of “N,N—(C₁₋₆alkyl)₂amino” include di-N-methylamino, di-(N-ethyl)amino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N,N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are methylaminocarbonyl and ethylaminocarbonyl. Examples of “N,N—(C₁₋₆alkyl)₂carbamoyl” are dimethylaminocarbonyl and methylethylaminocarbonyl. Examples of “C₁₋₆alkylsulphonylamino” include methylsulphonylamino, isopropylsulphonylamino and t-butylsulphonylamino. Examples of “C₁₋₆alkylsulphonyl” include methylsulphonyl, isopropylsulphonyl and t-butylsulphonyl.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

An in vivo hydrolysable ester of a compound of the formula (I) containing carboxy or hydroxy group is, for example, a pharmaceutically acceptable ester which is hydrolysed in the human or animal body to produce the parent acid or alcohol. Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

An in vivo hydrolysable ester of a compound of the formula (I) containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

Some compounds of the formula (I) may have chiral centres and/or geometric isomeric centres (E- and Z-isomers), and it is to be understood that the invention encompasses all such optical, diastereoisomers and geometric isomers that possess CDK inhibitory activity.

The invention relates to any and all tautomeric forms of the compounds of the formula (I) that possess CDK inhibitory activity. In particular the skilled reader will appreciate that when R³ is hydrogen, the imidazole ring as drawn in formula (I) may tautomerise.

It is also to be understood that certain compounds of the formula (I) can exist in solvated as well as unsolvated forms such as, for example, hydrated forms. It is to be understood that the invention encompasses all such solvated forms which possess CDK inhibitory activity.

Particular values of variable groups are as follows. Such values may be used where appropriate with any of the definitions, claims or embodiments defined hereinbefore or hereinafter.

R¹ is carbamoyl, a group -R⁶-R⁷ or a nitrogen linked 4-7 membered saturated ring which optionally contains an additional nitrogen or oxygen atom; wherein if said ring contains an additional nitrogen atom that nitrogen may be optionally substituted by R⁹;

R⁶ is —C(O)—, —C(O)N(R¹⁷)— or —S(O)ᵣ—; wherein R¹⁷ is hydrogen or C₁₋₆alkyl and r is 0 or 2;

R⁷ is selected from C₁₋₆alkyl, carbocyclyl or heterocyclyl; wherein R⁷ may be optionally substituted on carbon by one or more K 2; and wherein if said heterocyclyl contains an —NH— moiety that nitrogen may be optionally substituted by a group selected from R²³;

R²² is N,N—(C₁₋₆alkyl)₂amino;

R⁹ and R²³ are independently selected from C₁₋₄alkyl or C₁₋₄alkanoyl; wherein R⁹ and R²³ independently of each other may be optionally substituted on carbon by one or more R³⁵; and

R³⁵ is hydroxy.

R¹ is carbamoyl, a group -R⁶-R⁷ or a nitrogen linked 4-7 membered saturated ring which optionally contains an additional nitrogen or an oxygen atom wherein if said ring contains an additional nitrogen atom that nitrogen may be optionally substituted by R⁹; wherein

R⁶ is —C(O)N(R¹⁷)— or —S(O)ᵣ—; wherein R¹⁷ is hydrogen or C₁₋₆alkyl and r is 0-2;

R⁷ is selected from C₁₋₆alkyl or carbocyclyl;

R⁹ is selected from C₁₋₄alkyl and C₁₋₄alkanoyl; wherein R⁹ may be optionally substituted on carbon by one or more R³⁵; and

R³⁵ is hydroxy.

R¹ is carbamoyl, a group -R⁶-R⁷, piperazinyl or morpholino; wherein said piperazinyl may be optionally substituted on nitrogen by R⁹;

R⁶ is —C(O)—, —C(O)N(R¹⁷)— or —S(O)ᵣ—; wherein R¹⁷ is hydrogen or methyl and r is 0 or 2;

R⁷ is selected from methyl, ethyl, cyclopropyl, pyrrolidinyl, piperidinyl, 1,4-diazepanyl or piperazinyl; wherein R⁷ may be optionally substituted on carbon by one or more R²²; and wherein said piperidinyl, piperazinyl or 1,4-diazepanyl may be optionally substituted on nitrogen by a group selected from R²³;

R²² is dimethylamino;

R⁹ and R²³ are independently selected from methyl, acetyl or propionyl; wherein R⁹ and R²³ independently of each other may be optionally substituted on carbon by one or more R³⁵; and

R³⁵ is hydroxy.

R¹ is carbamoyl, a group -R⁶-R⁷ or a nitrogen linked 4-7 membered saturated ring which optionally contains an oxygen atom; wherein

R⁶ is —C(O)N(R¹⁷)— or —S(O)ᵣ—; wherein R¹⁷ is hydrogen or C₁₋₆alkyl and r is 0-2.

R⁷ is selected from C₁₋₆alkyl or carbocyclyl.

R¹ is carbamoyl, a group -R⁶-R⁷, morpholino or piperazin-1-yl; wherein

R⁶ is —C(O)N(R¹⁷)— or —S(O)ᵣ—; wherein R¹⁷ is hydrogen or C₁₋₆alkyl and r is 0-2;

R⁷ is selected from C₁₋₆alkyl or cyclopropyl.

R⁹ is selected from C₁₋₄alkyl and C₁₋₄alkanoyl; wherein R⁹ may be optionally substituted on carbon by one or more R³⁵; and

R³⁵ is hydroxy.

R¹ is carbamoyl, a group -R⁶-R⁷ or morpholino; wherein

R⁶ is —C(O)N(R¹⁷)— or —S(O)ᵣ—; wherein R¹⁷ is hydrogen or C₁₋₆alkyl and r is 0-2.

R⁷ is selected from C₁₋₆alkyl or cyclopropyl.

R¹ is carbamoyl, N-methylcarbamoyl, N-ethylcarbamoyl, N,N-dimethylcarbamoyl, methylthio, mesyl, N-cyclopropylcarbamoyl, morpholino, piperazin-1-yl, 4-acetylpiperazin-1-yl, 4-methylpiperazin-1-yl, 4-(2-hydroxypropionyl)piperazin-1-yl or 4-(2-hydroxyacetyl)piperazin-1-yl.

R¹ is carbamoyl, morpholino, N-methylcarbamoyl, N,N-dimethylcarbamoyl, methylthio, mesyl, N-cyclopropylcarbamoyl, N-ethylcarbamoyl, piperazin-1-yl, 4-((R)-2-hydroxypropionyl)piperazin-1-yl, 4-((S)-2-hydroxypropionyl)piperazin-1-yl, 4-(2-hydroxyacetyl)piperazin-1-yl, 4-(acetyl)piperazin-1-yl, morpholinocarbonyl, 4-methylpiperazin-1-ylcarbonyl, 4-methyl-1,4-diazepanylcarbonyl, N-(1-methylpiperidin-4-yl)carbamoyl and (S)-3-dimethylaminopyrrolidin-1-ylcarbonyl.

R¹ is carbamoyl, N-methylcarbamoyl, N-ethylcarbamoyl, N,N-dimethylcarbamoyl, methylthio, mesyl, N-cyclopropylcarbamoyl or morpholino.

X¹ is ═N—.

X² is ═N—.

X³ is ═N—.

X⁴ is ═N—.

X¹ is ═N— and X³, X² and X⁴ are independently selected from ═C(R¹⁰)—.

X³ is ═N— and X¹, X² and X⁴ are independently selected from ═C(R¹⁰)—.

X⁴ is ═N— and X¹, X² and X³ are independently selected from ═C(R¹⁰)—.

X¹ and X⁴ are ═N— and X² and X³ are independently selected from ═C(R¹⁰)—.

X⁴ is ═N— and X¹, X² and X³ are independently selected from ═C(R¹⁰)—; or X³ is ═N— and X¹, X² and X⁴ are independently selected from ═C(R¹⁰)—; or X¹ is ═N— and X³, X² and X⁴ are independently selected from ═C(R¹⁰)—; or X¹ and X⁴ are ═N— and X² and X³ are independently selected from ═C(R¹⁰)—; wherein

R¹⁰ is selected from halo or C₁₋₆alkyl.

X⁴ is ═N— and X¹, X² and X³ are independently selected from ═C(R¹⁰)—; or X¹ is ═N— and X³, X² and X⁴ are independently selected from ═C(R¹⁰)—; or X¹ and X⁴ are ═N— and X² and X³ are independently selected from ═C(R¹⁰)—; wherein

R¹⁰ is selected from hydrogen, halo or C₁₋₆alkyl.

X⁴ is ═N— and X¹, X² and X³ are independently selected from ═C(R¹⁰)—; or X³ is ═N— and X³, X² and X⁴ are independently selected from ═C(R¹⁰)—; or X¹ is ═N— and X³, X² and X⁴ are independently selected from ═C(R¹⁰)—; or X¹ and X⁴ are ═N— and X² and X³ are independently selected from ═C(R¹⁰)—; wherein

R¹⁰ is selected from chloro or methyl.

X⁴ is ═N— and X¹, X² and X³ are independently selected from ═C(R¹⁰)—; or X¹ is ═N— and X³, X² and X⁴ are independently selected from ═C(R¹⁰)—; or X¹ and X⁴ are ═N— and X² and X³ are independently selected from ═C(R¹⁰)—; wherein

R¹⁰ is selected from hydrogen, chloro or methyl.

R¹⁰ is not hydrogen.

R² is halo.

R² is fluoro.

R² is chloro.

R² is fluoro or chloro.

R² is 6-fluoro.

n is 0 or 1.

n is 0.

n is 1.

R³ is C₁₋₆alkyl or carbocyclyl.

R³ is C₁₋₆alkyl.

R³ is isopropyl or cyclopentyl.

R³ is isopropyl.

R⁴ is C₁₋₆alkyl.

R⁴ is C₁₋₆alkyl or carbocyclyl.

R⁴ is methyl.

R⁴ is methyl or cyclopropyl.

R⁵ is hydrogen.

Therefore in a further aspect of the invention there is provided a compound of formula (I) (as depicted above) wherein:

R¹ is carbamoyl, a group -R⁶-R⁷ or a nitrogen linked 4-7 membered saturated ring which optionally contains an additional nitrogen or oxygen atom; wherein if said ring contains an additional nitrogen atom that nitrogen may be optionally substituted by R⁹;

X⁴ is ═N— and X¹, X² and X³ are independently selected from ═C(R¹⁰)—; or X¹ is ═N— and X³, X² and X⁴ are independently selected from ═C(R¹⁰)—; or X¹ and X⁴ are ═N— and X² and X³ are independently selected from ═C(R¹⁰)—;

R² is halo;

n is 0 or 1;

R³ is C₁₋₆alkyl or carbocyclyl;

R⁴ is C₁₋₆alkyl or carbocyclyl;

R⁵ is hydrogen;

R⁶ is —C(O)—, —C(O)N(R¹⁷)— or —S(O)ᵣ—; wherein R¹⁷ is hydrogen or C₁₋₆alkyl and r is 0 or 2;

R⁷ is selected from C₁₋₆alkyl, carbocyclyl or heterocyclyl; wherein R⁷ may be optionally substituted on carbon by one or more R²; and wherein if said heterocyclyl contains an —NH— moiety that nitrogen may be optionally substituted by a group selected from R²³;

R⁹ and R²³ are independently selected from C₁₋₄alkyl or C₁₋₄alkanoyl; wherein R⁹ and R²³ independently of each other may be optionally substituted on carbon by one or more R³⁵;

R¹⁰ is selected from hydrogen, halo or C₁₋₆alkyl;

R²² is N,N—(C₁₋₆alkyl)₂amino;

R³⁵ is hydroxy;

or a pharmaceutically acceptable salt or an in vivo hydrolysable ester thereof.

Therefore in a further aspect of the invention there is provided a compound of formula (I) (as depicted above) wherein:

R¹ is carbamoyl, a group -R⁶-R⁷ or a nitrogen linked 4-7 membered saturated ring which optionally contains an oxygen atom;

X⁴ is ═N— and X¹, X² and X³ are independently selected from ═C(R¹⁰)—; or X³ is ═N— and X¹, X² and X⁴ are independently selected from ═C(R¹⁰)—; or X¹ is ═N— and X³, X² and X⁴ are independently selected from ═C(R¹⁰)—; or X¹ and X⁴ are ═N— and X² and X³ are independently selected from ═C(R¹⁰)—;

R² is halo;

n is 0 or 1;

R³ is C₁₋₆alkyl;

R⁴ is C₁₋₆alkyl;

R⁵ is hydrogen;

R⁶ is —C(O)N(R¹⁷)— or —S(O)ᵣ—; wherein R¹⁷ is hydrogen or C₁₋₆alkyl and r is 0-2; and

R⁷ is selected from C₁₋₆alkyl or carbocyclyl;

R¹⁰ is selected from hydrogen, halo or C₁₋₆alkyl;

or a pharmaceutically acceptable salt or an in vivo hydrolysable ester thereof.

Therefore in a further aspect of the invention there is provided a compound of formula (I) (as depicted above) wherein:

R¹ is carbamoyl, a group -R⁶-R⁷ or a nitrogen linked 4-7 membered saturated ring which optionally contains an oxygen atom;

X⁴ is ═N— and X¹, X² and X³ are independently selected from ═C(R¹⁰)—; or X³ is ═N— and X¹, X² and X⁴ are independently selected from ═C(R¹⁰)—; or X¹ is ═N— and X³, X² and X⁴ are independently selected from ═C(R¹⁰)—; or X¹ and X⁴ are ═N— and X² and X³ are independently selected from ═C(R¹⁰)—;

R² is halo;

n is 0 or 1;

R³ is C₁₋₆alkyl;

R⁴ is C₁₋₆alkyl;

R⁵ is hydrogen;

R⁶ is —C(O)N(R¹⁷)— or —S(O)ᵣ—; wherein R¹⁷ is hydrogen or C₁₋₆alkyl and r is 0-2; and

R⁷ is selected from C₁₋₆alkyl or carbocyclyl;

R¹⁰ is selected from halo or C₁₋₆alkyl;

or a pharmaceutically acceptable salt or an in vivo hydrolysable ester thereof.

Therefore in a further aspect of the invention there is provided a compound of formula (I) (as depicted above) wherein:

R¹ is carbamoyl, a group -R⁶-R⁷ or a nitrogen linked 4-7 membered saturated ring which optionally contains an oxygen atom;

X⁴ is ═N— and X¹, X² and X³ are independently selected from ═C(R¹⁰)—; or X³ is ═N— and X¹, X² and X⁴ are independently selected from ═C(R¹⁰)—; or X¹ is ═N— and X³, X² and X⁴ are independently selected from ═C(R¹⁰)—; or X¹ and X⁴ are ═N— and X² and X³ are independently selected from ═C(R¹⁰)—;

R² is halo;

n is 0 or 1;

R³ is C₁₋₆alkyl;

R⁴ is C₁₋₆alkyl;

R⁵ is hydrogen;

R⁶ is —C(O)N(R¹⁷)— or —S(O)ᵣ—; wherein R¹⁷ is hydrogen or C₁₋₆alkyl and r is 0-2; and

R⁷ is selected from C₁₋₆alkyl or carbocyclyl;

R¹⁰ is selected from hydrogen, halo or C₁₋₆alkyl;

or a pharmaceutically acceptable salt or an in vivo hydrolysable ester thereof.

Therefore in a further aspect of the invention there is provided a compound of formula (I) (as depicted above) wherein

R¹ is carbamoyl, N-methylcarbamoyl, N-ethylcarbamoyl, N,N-dimethylcarbamoyl, methylthio, mesyl, N-cyclopropylcarbamoyl or morpholino;

X⁴ is ═N— and X¹, X² and X³ are independently selected from ═C(R¹⁰)—; or X³ is ═N— and X¹, X² and X⁴ are independently selected from ═C(R¹⁰)—; or X¹ is ═N— and X³, X² and X⁴ are independently selected from ═C(R¹⁰)—; or X¹ and X⁴ are ═N— and X² and X³ are independently selected from ═C(R¹⁰)—;

R² is fluoro;

n is 0 or 1;

R³ is isopropyl;

R⁴ is methyl;

R⁵ is hydrogen; and

R¹⁰ is selected from chloro or methyl;

or a pharmaceutically acceptable salt or an in vivo hydrolysable ester thereof.

Therefore in a further aspect of the invention there is provided a compound of formula (I) (as depicted above) wherein

R¹ is carbamoyl, N-methylcarbamoyl, N-ethylcarbamoyl, N,N-dimethylcarbamoyl, methylthio, mesyl, N-cyclopropylcarbamoyl or morpholino;

X⁴ is ═N— and X¹, X² and X³ are independently selected from ═C(R¹⁰)—; or X³ is ═N— and X¹, X² and X⁴ are independently selected from ═C(R¹⁰)—; or X¹ is ═N— and X³, X² and X⁴ are independently selected from ═C(R¹⁰)—; or X¹ and X⁴ are ═N— and X² and X³ are independently selected from ═C(R¹⁰)—;

R² is fluoro;

n is 0 or 1;

R³ is isopropyl;

R⁴ is methyl;

R⁵ is hydrogen; and

R¹⁰ is selected from hydrogen, chloro or methyl;

or a pharmaceutically acceptable salt or an in vivo hydrolysable ester thereof.

Therefore in a further aspect of the invention there is provided a compound of formula (I) (as depicted above) wherein

R¹ is carbamoyl, morpholino, N-methylcarbamoyl, N,N-dimethylcarbamoyl, methylthio, mesyl, N-cyclopropylcarbamoyl, N-ethylcarbamoyl, piperazin-1-yl, 4-((R)-2-hydroxypropionyl)piperazin-1-yl, 4-((S)-2-hydroxypropionyl)piperazin-1-yl, 4-(2-hydroxyacetyl)piperazin-1-yl, 4-(acetyl)piperazin-1-yl, morpholinocarbonyl, 4-methylpiperazin-1-ylcarbonyl, 4-methyl-1,4-diazepanylcarbonyl, N-(1-methylpiperidin-4-yl)carbamoyl and (S)-3-dimethylaminopyrrolidin-1-ylcarbonyl;

X⁴ is ═N— and X¹, X² and X³ are independently selected from ═C(R¹⁰)—; or X¹ is ═N— and X³, X² and X⁴ are independently selected from ═C(R¹⁰)—; or X¹ and X⁴ are ═N— and X² and X³ are independently selected from ═C(R¹⁰)—;

R² is fluoro or chloro;

n is 0 or 1;

R³ is isopropyl or cyclopentyl;

R⁴ is methyl or cyclopropyl;

R⁵ is hydrogen;

R¹⁰ is selected from hydrogen, chloro or methyl;

or a pharmaceutically acceptable salt or an in vivo hydrolysable ester thereof.

In another aspect of the invention, preferred compounds of the invention are any one of the Examples or a pharmaceutically acceptable salt or an in vivo hydrolysable ester thereof.

In another aspect of the invention, preferred compounds of the invention are any one of Examples 4, 5, 6, 7, 8, 9, 21, 29, 33 or 37, or a pharmaceutically acceptable salt or an in vivo hydrolysable ester thereof.

Preferred aspects of the invention are those which relate to the compound of formula (I) or a pharmaceutically acceptable salt thereof.

Another aspect of the present invention provides a process for preparing a compound of formula (I) or a pharmaceutically acceptable salt or an in vivo hydrolysable ester thereof which process (wherein variable groups are, unless otherwise specified, as defined in formula (I)) comprises of:

Process a) reaction of a pyrimidine of formula (II):

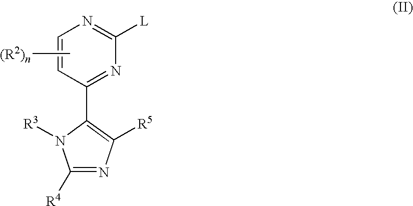

wherein L is a displaceable group; with an aniline of formula (III):

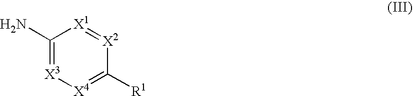

or
Process b) reacting a compound of formula (IV):

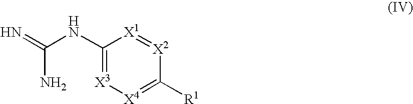

with a compound of formula (V):

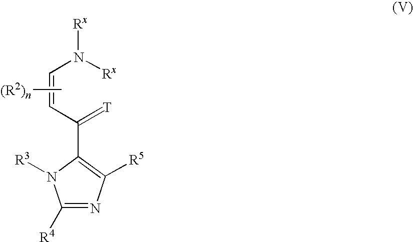

wherein T is O or S; R^{x} may be the same or different and is selected from C₁₋₆alkyl; or
Process c) for compounds of formula (I) wherein R¹ is carbamoyl or —C(O)N(R¹⁷)(R⁷) reacting an acid of formula (VI):

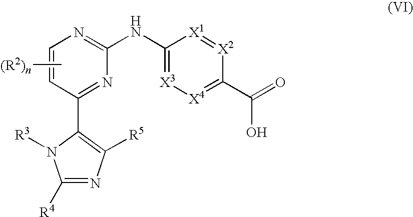

or an activated derivative thereof; with an amine of formula (VII):

HNR⁷′R¹⁷ (VII)

wherein R⁷′ is R⁷ or hydrogen; or
Process d) for compounds of formula (I); reacting a pyrimidine of formula (VIII):

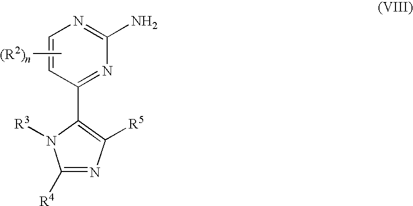

with a compound of formula (IX):

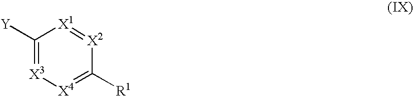

where Y is a displaceable group; and thereafter if necessary:
i) converting a compound of the formula (I) into another compound of the formula (I);
ii) removing any protecting groups;
iii) forming a pharmaceutically acceptable salt or in vivo hydrolysable ester.

L is a displaceable group, suitable values for L are for example, a halogeno or sulphonyloxy group, for example a chloro, bromo, methanesulphonyloxy or toluene-4-sulphonyloxy group.

Y is a displaceable group, suitable values for Y are for example, a halogeno or sulphonyloxy group, for example a bromo, iodo or trifluoromethanesulphonyloxy group. Preferably Y is iodo.

Specific reaction conditions for the above reactions are as follows.

Process a) Pyrimidines of formula (II) and anilines of formula (III) may be reacted together:
i) in the presence of a suitable solvent for example a ketone such as acetone or an alcohol such as ethanol or butanol or an aromatic hydrocarbon such as toluene or N-methyl pyrrolidine, optionally in the presence of a suitable acid for example an inorganic acid such as hydrochloric acid or sulphuric acid, or an organic acid such as acetic acid or formic acid (or a suitable Lewis acid) and at a temperature in the range of 0° C. to reflux, preferably reflux; or
ii) under standard Buchwald conditions (for example see J. Am. Chem. Soc., 118, 7215; J. Am. Chem. Soc., 119, 8451; J. Org. Chem., 62, 1568 and 6066) for example in the presence of palladium acetate, in a suitable solvent for example an aromatic solvent such as toluene, benzene or xylene, with a suitable base for example an inorganic base such as caesium carbonate or an organic base such as potassium-t-butoxide, in the presence of a suitable ligand such as 2,2′-bis(diphenylphosphino)-1,1′-binaphthyl and at a temperature in the range of 25 to 80° C.

Pyrimidines of the formula (II) where L is chloro may be prepared according to Scheme 1:

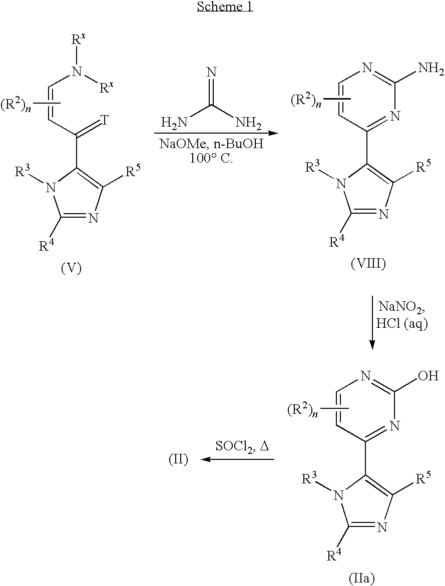

Anilines of formula (III) are commercially available compounds, or they are known in the literature, or they are prepared by standard processes known in the art.

Process b) Compounds of formula (IV) and compounds of formula (V) are reacted together in a suitable solvent such as N-methylpyrrolidinone or butanol at a temperature in the range of 100-200° C., preferably in the range of 150-170° C. The reaction is preferably conducted in the presence of a suitable base such as, for example, sodium hydride, sodium methoxide or potassium carbonate.

Compounds of formula (V) may be prepared according to Scheme 2:

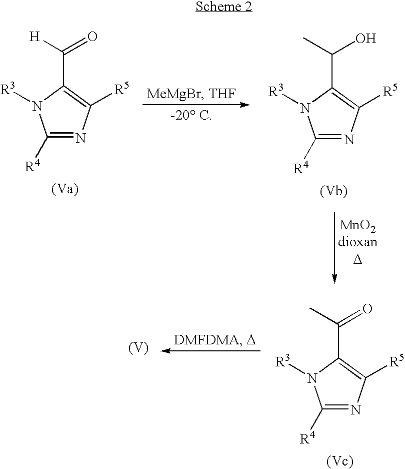

Compounds of formula (IV) and (Va) are commercially available compounds, or they are known in the literature, or they are prepared by standard processes known in the art.

Process c) Acids and amines may be coupled together in the presence of a suitable coupling reagent. Standard peptide coupling reagents known in the art can be employed as suitable coupling reagents, or for example carbonyldiimidazole and dicyclohexyl-carbodiimide, optionally in the presence of a catalyst such as dimethylaminopyridine or 4-pyrrolidinopyridine, optionally in the presence of a base for Example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

Compounds of formula (VI) may be prepared by adapting Process a), b) or c).

Amines of formula (VII) are commercially available compounds, or they are known in the literature, or they are prepared by standard processes known in the art.

Process d) Compounds of formula (VIII) and amines of formula (IX) may be reacted together under standard Buchwald conditions as described in Process a.

The synthesis of compounds of formula (VIII) is described in Scheme 1.

Compounds of formula (IX) are commercially available compounds, or they are known in the literature, or they are prepared by standard processes known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an allylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses anti-cell-proliferation activity such as anti-cancer activity which is believed to arise from the CDK inhibitory activity of the compound. These properties may be assessed, for example, using the procedure set out below:—

### Assay

The following abbreviations have been used:—

HEPES is N-[2-Hydroxyethyl]piperazine-N-[2-ethanesulfonic acid]

### DTT is Dithiothreitol

PMSF is Phenylmethylsulphonyl fluoride

The compounds were tested in an in vitro kinase assay in 96 well format using Scintillation Proximity Assay (SPA—obtained from Amersham) for measuring incorporation of [γ-33-P]-Adenosine Triphosphate into a test substrate (GST-Retinoblastoma protein; GST-Rb). In each well was placed the compound to be tested (diluted in DMSO and water to correct concentrations) and in control wells either roscovitine as an inhibitor control or DMSO as a positive control.

Approximately 0.2 μl of CDK2/Cyclin E partially-purified enzyme (amount dependent on enzyme activity) diluted in 25 μl incubation buffer was added to each well then 20 μl of GST-Rb/ATP/ATP33 mixture (containing 0.5 kg GST-Rb and 0.2 μM ATP and 0.14 μCi [γ-33-P]-Adenosine Triphosphate in incubation buffer), and the resulting mixture shaken gently, then incubated at room temperature for 60 minutes.

To each well was then added 150 μL stop solution containing (0.8 mg/well of Protein A-PVT SPA bead (Amersham)), 20 μM/well of Anti-Glutathione Transferase, Rabbit IgG (obtained from Molecular Probes), 61 mM EDTA and 50 mM HEPES pH 7.5 containing 0.05% sodium azide.

The plates were sealed with Topseal-S plate sealers, left for two hours then spun at 2500 rpm, 1124×g., for 5 minutes. The plates were read on a Topcount for 30 seconds per well.

The incubation buffer used to dilute the enzyme and substrate mixes contained 50 mM HEPES pH7.5, 10 mM MnCl₂, 1 mM DTT, 100 M Sodium vanadate, 100 μM NaF, 10 mM Sodium Glycerophosphate, BSA (1 mg/ml final).

### Test Substrate

In this assay only part of the retinoblastoma protein (Science 1987 Mar. 13; 235(4794):1394-1399; Lee W. H., Bookstein R., Hong F., Young L. J., Shew J. Y., Lee E. Y.) was used, fused to a GST tag. PCR of retinoblastoma gene encoding amino acids 379-928 (obtained from retinoblastoma plasmid ATCC pLRbRNL) was performed, and the sequence cloned into pGEx 2T fusion vector (Smith D. B. and Johnson, K. S. Gene 67, 31 (1988); which contained a tac promoter for inducible expression, internal lac I^{q} gene for use in any E. Coli host, and a coding region for thrombin cleavage—obtained from Pharmacia Biotech) which was used to amplify amino acids 792-928. This sequence was again cloned into pGEx 2T.

The retinoblastoma 792-928 sequence so obtained was expressed in E. Coli (BL21 (DE3) pLysS cells) using standard inducible expression techniques, and purified as follows.

E. coli paste was resuspended in 10 ml/g of NETN buffer (50 mM Tris pH 7.5, 120 mM NaCl, 1 mM EDTA, 0.5% v/v NP-40, 1 mM PMSF, 1 ug/ml leupeptin, 1 ug/ml aprotinin and 1 ug/ml pepstatin) and sonicated for 2×45 seconds per 100 ml homogenate. After centrifugation, the supernatant was loaded onto a 10 ml glutathione Sepharose column (Pharmacia Biotech, Herts, UK), and washed with NETN buffer. After washing with kinase buffer (50 mM HEPES pH 7.5, 10 mM MgCl2, 1 mM DTT, 1 mM PMSF, 1 ug/ml leupeptin, 1 ug/ml aprotinin and 1 ug/ml pepstatin) the protein was eluted with 50 mM reduced glutathione in kinase buffer. Fractions containing GST-Rb(792-927) were pooled and dialysed overnight against kinase buffer. The final product was analysed by Sodium Dodeca Sulfate (SDS) PAGE (Polyacrylamide gel) using 8-16% Tris-Glycine gels (Novex, San Diego, USA).

### CDK2 and Cyclin E

The open reading frames of CDK2 and Cyclin E were isolated by reverse transcriptase-PCR using HeLa cell and activated T cell mRNA as a template and cloned into the insect expression vector pVL1393 (obtained from Invitrogen 1995 catalogue number: V1392-20). CDK2 and cyclin E were then dually expressed [using a standard virus Baculogold co-infection technique] in the insect SF21 cell system (Spodoptera Frugiperda cells derived from ovarian tissue of the Fall Army Worm—commercially available).

### Example Production of Cyclin E/CDK2

The following Example provides details of the production of Cyclin E/CDK2 in SF21 cells (in TC100+10% FBS(TCS)+0.2% Pluronic) having dual infection MOI 3 for each virus of Cyclin E & CDK2.

SF21 cells grown in a roller bottle culture to 2.33×10⁶ cells/ml were used to inoculate 10×500 ml roller bottles at 0.2×10E6 cells/ml. The roller bottles were incubated on a roller rig at 28° C.

After 3 days (72 hrs.) the cells were counted, and the average from 2 bottles found to be 1.86×10E6 cells/ml. (99% viable). The cultures were then infected with the dual viruses at an MOI 3 for each virus.

The viruses were mixed together before addition to the cultures, and the cultures returned to the roller rig 28° C.

After 2 days (48 hrs.) post infection the 5 Litres of culture was harvested. The total cell count at harvest was 1.58×10E6 cells/ml. (99% viable). The cells were spun out at 2500 rpm, 30 mins., 4° C. in Heraeus Omnifuge 2.0 RS in 250 ml. lots. The supernatant was discarded.

### Partial Co-Purification of CDK2 and Cyclin E

Sf21 cells were resuspended in lysis buffer (50 mM Tris pH 8.2, 10 mM MgCl₂, 1 mM DTT, 10 mM glycerophosphate, 0.1 mM sodium orthovanadate, 0.1 mM NaF, 1 mM PMSF, 1 ug/ml leupeptin and 1 ug/ml aprotinin) and homogenised for 2 minutes in a 10 ml Dounce homgeniser. After centrifugation, the supernatant was loaded onto a Poros HQ/M 1.4/100 anion exchange column (PE Biosystems, Hertford, UK). CDK2 and Cyclin E were coeluted at the beginning of a 0-1M NaCl gradient (run in lysis buffer minus protease inhibitors) over 20 column volumes. Co-elution was checked by western blot using both anti-CDK2 and anti-Cyclin E antibodies (Santa Cruz Biotechnology, California, US).

By analogy, assays designed to assess inhibition of CDK1 and CDK4 may be constructed. CDK2 (EMBL Accession No. X62071) may be used together with Cyclin A or Cyclin E (see EMBL Accession No. M73812), and further details for such assays are contained in PCT International Publication No. WO99/21845, the relevant Biochemical & Biological Evaluation sections of which are hereby incorporated by reference.

Although the pharmacological properties of the compounds of the formula (I) vary with structural change, in general activity possessed by compounds of the formula (I) may be demonstrated at IC₅₀ concentrations or doses in the range 250 μM to 1 nM.

When tested in the above in-vitro assay the CDK2 inhibitory activity of Example 22 was measured as IC₅₀=0.0490 μM.

The in vivo activity of the compounds of the present invention may be assessed by standard techniques, for example by measuring inhibition of cell growth and assessing cytotoxicity.

Inhibition of cell growth may be measured by staining cells with Sulforhodamine B (SRB), a fluorescent dye that stains proteins and therefore gives an estimation of amount of protein (i.e. cells) in a well (see Boyd, M. R. (1989) Status of the NCI preclinical antitumour drug discovery screen. Prin. Prac Oncol 10:1-12). Thus, the following details are provided of measuring inhibition of cell growth:—

Cells may be plated in appropriate medium in a volume of 100 ml in 96 well plates; media can be Dulbecco's Modified Eagle media for MCF-7, SK-UT-1B and SK-UT-1. The cells may be allowed to attach overnight, then inhibitor compounds added at various concentrations in a maximum concentration of 1% DMSO (v/v). A control plate can be assayed to give a value for cells before dosing. Cells can be incubated at 37° C., (5% CO₂) for three days.

At the end of three days TCA may be added to the plates to a final concentration of 16% (v/v). Plates may then be incubated at 4° C. for 1 hour, the supernatant removed and the plates washed in tap water. After drying, 100 ml SRB dye (0.4% SRB in 1% acetic acid) may be added for 30 minutes at 37° C. Excess SRB can be removed and the plates washed in 1% acetic acid. The SRB bound to protein can be solubilised in 10 mM Tris pH7.5 and shaken for 30 minutes at room temperature. The ODs may be read at 540 nm, and the concentration of inhibitor causing 50% inhibition of growth determined from a semi-log plot of inhibitor concentration versus absorbance. The concentration of compound that reduced the optical density to below that obtained when the cells may be plated at the start of the experiment gave the value for toxicity.

Typical IC₅₀ values for compounds of the invention when tested in the SRB assay will be in the range 1 mM to 1 nM.

According to a further aspect of the invention there is provided a pharmaceutical composition which comprises a pyrimidine derivative of the formula (I), or a pharmaceutically acceptable salt or in vivo hydrolysable ester thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 5-5000 mg per square meter body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt or in vivo hydrolysable ester thereof, as defined hereinbefore for use in a method of treatment of the human or animal body by therapy.

We have found that the compounds defined in the present invention, or a pharmaceutically acceptable salt or in vivo hydrolysable ester thereof, are effective cell cycle inhibitors (anti-cell proliferation agents), which property is believed to arise from their CDK inhibitory properties. Accordingly the compounds of the present invention are expected to be useful in the treatment of diseases or medical conditions mediated alone or in part by CDK enzymes, i.e. the compounds may be used to produce a CDK inhibitory effect in a warm-blooded animal in need of such treatment. Thus the compounds of the present invention provide a method for treating the proliferation of malignant cells characterised by inhibition of CDK enzymes, i.e. the compounds may be used to produce an anti-proliferative effect mediated alone or in part by the inhibition of CDKs. Such a compound of the invention is expected to possess a wide range of anti-cancer properties as CDKs have been implicated in many common human cancers such as leukaemia and breast, lung, colon, rectal, stomach, prostate, bladder, pancreas and ovarian cancer. Thus it is expected that a compound of the invention will possess anti-cancer activity against these cancers. It is in addition expected that a compound of the present invention will possess activity against a range of leukaemias, lymphoid malignancies and solid tumours such as carcinomas and sarcomas in tissues such as the liver, kidney, prostate and pancreas. In particular such compounds of the invention are expected to slow advantageously the growth of primary and recurrent solid tumours of, for example, the colon, breast, prostate, lungs and skin. More particularly such compounds of the invention, or a pharmaceutically acceptable salt or in vivo hydrolysable ester thereof, are expected to inhibit the growth of those primary and recurrent solid tumours which are associated with CDKs, especially those tumours which are significantly dependent on CDKs for their growth and spread, including for example, certain tumours of the colon, breast, prostate, lung, vulva and skin.

It is further expected that a compound of the present invention will possess activity against other cell-proliferation diseases in a wide range of other disease states including leukaemias, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

Thus according to this aspect of the invention there is provided a compound of the formula (I), or a pharmaceutically acceptable salt or in vivo hydrolysable ester thereof, as defined hereinbefore for use as a medicament; and the use of a compound of the formula (I), or a pharmaceutically acceptable salt or in vivo hydrolysable ester thereof, as defined hereinbefore in the manufacture of a medicament for use in the production of a cell cycle inhibitory (anti-cell-proliferation) effect in a warm-blooded animal such as man. Particularly, an inhibitory effect is produced by preventing entry into, or progression through, the S phase by inhibition of CDK2 and CDK4, especially CDK2, and M phase by inhibition of CDK1.

According to a further feature of the invention, there is provided a compound of the formula (I), or a pharmaceutically acceptable salt or in vivo hydrolysable ester thereof, as defined herein before in the manufacture of a medicament for use in the treatment of cancers (solid tumours and leukaemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation, particularly in the treatment of cancers.

According to a further feature of this aspect of the invention there is provided a method for producing a cell cycle inhibitory (anti-cell-proliferation) effect in a warm-blooded animal, such as man, in need of such treatment which comprises administering to said animal an effective amount of a compound as defined immediately above. Particularly, an inhibitory effect is produced by preventing entry into, or progression through, the S phase by inhibition of CDK2 and CDK4, especially CDK2, and M phase by inhibition of CDK1.

According to a further feature of this aspect of the invention there is provided a method for producing a cell cycle inhibitory (anti-cell-proliferation) effect in a warm-blooded animal, such as man, in need of such treatment which comprises administering to said animal an effective amount of a compound of formula (I) or a pharmaceutically acceptable salt or in vivo hydrolysable ester thereof as defined herein before. Particularly, an inhibitory effect is produced by preventing entry into, or progression through, the S phase by inhibition of CDK2 and CDK4, especially CDK2, and M phase by inhibition of CDK1.

According to an additional feature of this aspect of the invention there is provided a method of treating cancers (solid tumours and leukaemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation, in a warm-blooded animal, such as man, in need of such treatment which comprises administering to said animal an effective amount of a compound of formula (I) or a pharmaceutically acceptable salt or in vivo hydrolysable ester thereof as defined herein before.

Particularly there is provided a method of treating cancer in a warm-blooded animal, such as man, in need of such treatment which comprises administering to said animal an effective amount of a compound of formula (I) or a pharmaceutically acceptable salt or in vivo hydrolysable ester thereof as defined herein before.

In a further aspect of the invention there is provided a pharmaceutical composition which comprises a compound of the formula (I), or a pharmaceutically acceptable salt or in vivo hydrolysable ester thereof, as defined herein before in association with a pharmaceutically-acceptable diluent or carrier for use in the production of a cell cycle inhibitory (anti-cell-proliferation) effect in a warm-blooded animal such as man.

Thus according to this aspect of the invention there is provided a compound of the formula (I), or a pharmaceutically acceptable salt or in vivo hydrolysable ester thereof, as defined hereinbefore for use as a medicament.

In a further aspect of the invention there is provided the use of a compound of the formula (I), or a pharmaceutically acceptable salt or in vivo hydrolysable ester thereof, as defined hereinbefore in the manufacture of a medicament for use in the production of a cell cycle inhibitory effect.

In a further aspect of the invention there is provided the use of a compound of the formula (I), or a pharmaceutically acceptable salt or in vivo hydrolysable ester thereof, as defined hereinbefore in the manufacture of a medicament for use in the production of an anti-cell-proliferation effect.

In a further aspect of the invention there is provided the use of a compound of the formula (I), or a pharmaceutically acceptable salt or in vivo hydrolysable ester thereof, as defined hereinbefore in the manufacture of a medicament for use in the production of a CDK2 or CDK4 inhibitory effect.

In a further aspect of the invention there is provided the use of a compound of the formula (I), or a pharmaceutically acceptable salt or in vivo hydrolysable ester thereof, as defined hereinbefore in the manufacture of a medicament for use in the treatment of cancer.

In a further aspect of the invention there is provided the use of a compound of the formula (I), or a pharmaceutically acceptable salt or in vivo hydrolysable ester thereof, as defined hereinbefore in the manufacture of a medicament for use in the treatment of leukaemia or lymphoid malignancies or cancer of the breast, lung, colon, rectum, stomach, liver, kidney, prostate, bladder, pancreas, vulva, skin or ovary.

According to a further feature of the invention, there is provided the use of a compound of the formula (I), or a pharmaceutically acceptable salt or in vivo hydrolysable ester thereof, as defined herein before in the manufacture of a medicament for use in the treatment of cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

In a further aspect of the invention there is provided a method of producing a cell cycle inhibitory effect, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of producing an anti-cell-proliferation effect, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of producing a CDK2 or CDK4 inhibitory effect, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of treating cancer, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of treating leukaemia or lymphoid malignancies or cancer of the breast, lung, colon, rectum, stomach, liver, kidney, prostate, bladder, pancreas, vulva, skin or ovary, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of treating cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use as a medicament.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the production of a cell cycle inhibitory effect.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the production of an anti-cell-proliferation effect.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the production of a CDK2 or CDK4 inhibitory effect.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the treatment of cancer.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the treatment of leukaemia or lymphoid malignancies or cancer of the breast, lung, colon, rectum, stomach, liver, kidney, prostate, bladder, pancreas, vulva, skin or ovary.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the treatment of cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

In a further aspect of the invention there is provided the use of a compound of the formula (I), or a pharmaceutically acceptable salt or in vivo hydrolysable ester thereof, as defined hereinbefore, in the production of a cell cycle inhibitory effect.

In a further aspect of the invention there is provided the use of a compound of the formula (I), or a pharmaceutically acceptable salt or in vivo hydrolysable ester thereof, as defined hereinbefore, in the production of an anti-cell-proliferation effect.

In a further aspect of the invention there is provided the use of a compound of the formula (I), or a pharmaceutically acceptable salt or in vivo hydrolysable ester thereof, as defined hereinbefore, in the production of a CDK2 or CDK4 inhibitory effect.

In a further aspect of the invention there is provided the use of a compound of the formula (I), or a pharmaceutically acceptable salt or in vivo hydrolysable ester thereof, as defined hereinbefore, in the treatment of cancer.

In a further aspect of the invention there is provided the use of a compound of the formula (I), or a pharmaceutically acceptable salt or in vivo hydrolysable ester thereof, as defined hereinbefore in the treatment of leukaemia or lymphoid malignancies or cancer of the breast, lung, colon, rectum, stomach, liver, kidney, prostate, bladder, pancreas, vulva, skin or ovary.

According to a further feature of the invention, there is provided the use of a compound of the formula (I), or a pharmaceutically acceptable salt or in vivo hydrolysable ester thereof, as defined herein before in the treatment of cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

In a further aspect of the invention there is provided a pharmaceutical composition which comprises a compound of the formula (I), or a pharmaceutically acceptable salt or in vivo hydrolysable ester thereof, as defined herein before in association with a pharmaceutically-acceptable diluent or carrier for use in the treatment of cancers (solid tumours and leukaemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation, in a warm-blooded animal such as man.

In a further aspect of the invention there is provided a pharmaceutical composition which comprises a compound of the formula (I), or a pharmaceutically acceptable salt or in vivo hydrolysable ester thereof, as defined herein before in association with a pharmaceutically-acceptable diluent or carrier for use in the treatment of cancer in a warm-blooded animal such as man.

Preventing cells from entering DNA synthesis by inhibition of essential S-phase initiating activities such as CDK2 initiation may also be useful in protecting normal cells of the body from toxicity of cycle-specific pharmaceutical agents. Inhibition of CDK2 or CDK4 will prevent progression into the cell cycle in normal cells which could limit the toxicity of cycle-specific pharmaceutical agents which act in S-phase, G2 or mitosis. Such protection may result in the prevention of hair loss normally associated with these agents.

Therefore in a further aspect of the invention there is provided a compound of formula (I) as defined above or a pharmaceutically acceptable salt or in vivo hydrolysable ester thereof for use as a cell protective agent.

Therefore in a further aspect of the invention there is provided a compound of formula (I) as defined above or a pharmaceutically acceptable salt or in vivo hydrolysable ester thereof for use in preventing hair loss arising from the treatment of malignant conditions with pharmaceutical agents.

Examples of pharmaceutical agents for treating malignant conditions that are known to cause hair loss include alkylating agents such as ifosfamide and cyclophosphamide; antimetabolites such as methotrexate, 5-fluorouracil, gemcitabine and cytarabine; vinca alkaloids and analogues such as vincristine, vinbalstine, vindesine, vinorelbine; taxanes such as paclitaxel and docetaxel; topoisomerase I inhibitors such as irintotecan and topotecan; cytotoxic antibiotics such as doxorubicin, daunorubicin, mitoxantrone, actinomycin-D and mitomycin; and others such as etoposide and tretinoin.

In another aspect of the invention, the compound of formula (I), or a pharmaceutically acceptable salt or in vivo hydrolysable ester thereof, may be administered in association with a one or more of the above pharmaceutical agents. In this instance the compound of formula (I) may be administered by systemic or non systemic means. Particularly the compound of formula (I) my may administered by non-systemic means, for example topical administration.

Therefore in an additional feature of the invention, there is provided a method of preventing hair loss during treatment for one or more malignant conditions with pharmaceutical agents, in a warm-blooded animal, such as man, which comprises administering to said animal an effective amount of a compound of formula (I), or a pharmaceutically acceptable salt or in vivo hydrolysable ester thereof.

In an additional feature of the invention, there is provided a method of preventing hair loss during treatment for one or more malignant conditions with pharmaceutical agents, in a warm-blooded animal, such as man, which comprises administering to said animal an effective amount of a compound of formula (I), or a pharmaceutically acceptable salt or in vivo hydrolysable ester thereof in simultaneous, sequential or separate administration with an effective amount of said pharmaceutical agent.

According to a further aspect of the invention there is provided a pharmaceutical composition for use in preventing hair loss arising from the treatment of malignant conditions with pharmaceutical agents which comprises a compound of formula (I), or a pharmaceutically acceptable salt or in vivo hydrolysable ester thereof, and said pharmaceutical agent, in association with a pharmaceutically acceptable diluent or carrier.

According to a further aspect of the present invention there is provided a kit comprising a compound of formula (I), or a pharmaceutically acceptable salt or in vivo hydrolysable ester thereof, and a pharmaceutical agent for treating malignant conditions that is known to cause hair loss.

According to a further aspect of the present invention there is provided a kit comprising:

a) a compound of formula (I), or a pharmaceutically acceptable salt or in vivo hydrolysable ester thereof, in a first unit dosage form;
b) a pharmaceutical agent for treating malignant conditions that is known to cause hair loss; in a second unit dosage form; and
c) container means for containing said first and second dosage forms.

According to another feature of the invention there is provided the use of a compound of the formula (I), or a pharmaceutically acceptable salt or in vivo hydrolysable ester thereof, in the manufacture of a medicament for the prevention of hair loss during treatment of malignant conditions with pharmaceutical agents.

According to a further aspect of the present invention there is provided a combination treatment for the prevention of hair loss comprising the administration of an effective amount of a compound of the formula (I), or a pharmaceutically acceptable salt or in vivo hydrolysable ester thereof, optionally together with a pharmaceutically acceptable diluent or carrier, with the simultaneous, sequential or separate administration of an effective amount of a pharmaceutical agent for treatment of malignant conditions to a warm-blooded animal, such as man.

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

The CDK inhibitory activity defined hereinbefore may be applied as a sole therapy or may involve, in addition to a compound of the invention, one or more other substances and/or treatments. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the cell cycle inhibitory treatment defined hereinbefore may be: surgery, radiotherapy or chemotherapy. Such chemotherapy may cover three main categories of therapeutic agent:

(i) other cell cycle inhibitory agents that work by the same or different mechanisms from those defined hereinbefore;
(ii) cytostatic agents such as antioestrogens (for example tamoxifen, toremifene, raloxifene, droloxifene, iodoxyfene), progestogens (for example megestrol acetate), aromatase inhibitors (for example anastrozole, letrazole, vorazole, exemestane), antiprogestogens, antiandrogens (for example flutamide, nilutamide, bicalutamide, cyproterone acetate), LHRH agonists and antagonists (for example goserelin acetate, luprolide), inhibitors of testosterone 5α-dihydroreductase (for example finasteride), anti-invasion agents (for example metalloproteinase inhibitors like marimastat and inhibitors of urokinase plasminogen activator receptor function) and inhibitors of growth factor function, (such growth factors include for example platelet derived growth factor and hepatocyte growth factor such inhibitors include growth factor antibodies, growth factor receptor antibodies, tyrosine kinase inhibitors and serine/threonine kinase inhibitors); and
(iii) antiproliferative/antineoplastic drugs and combinations thereof, as used in medical oncology, such as antimetabolites (for example antifolates like methotrexate, fluoropyrimidines like 5-fluorouracil, purine and adenosine analogues, cytosine arabinoside); antitumour antibiotics (for example anthracyclines like doxorubicin, daunomycin, epirubicin and idarubicin, mitomycin-C, dactinomycin, mithramycin); platinum derivatives (for example cisplatin, carboplatin); alkylating agents (for example nitrogen mustard, melphalan, chlorambucil, busulphan, cyclophosphamide, ifosfamide, nitrosoureas, thiotepa); antimitotic agents (for example vinca alkaloids like vincristine and taxoids like taxol, taxotere); topoisomerase inhibitors (for example epipodophyllotoxins like etoposide and teniposide, amsacrine, topotecan). According to this aspect of the invention there is provided a pharmaceutical product comprising a compound of the formula (I) as defined hereinbefore and an additional anti-tumour substance as defined hereinbefore for the conjoint treatment of cancer.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non limiting examples in which, unless stated otherwise:

(i) temperatures are given in degrees Celsius (° C.); operations were carried out at room or ambient temperature, that is, at a temperature in the range of 18-25° C.;
(ii) organic solutions were dried over anhydrous magnesium sulphate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and reaction times are given for illustration only;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, unless otherwise stated, given in parts per million (ppm) relative to tetramethylsilane (TMS) as an internal standard, determined at 300 MHz using perdeuterio dimethyl sulphoxide (DMSO-d₆) as solvent unless otherwise indicated; ¹⁷F NMR was also measured in DMSO-d₆ unless otherwise stated;
(viii) chemical symbols have their usual meanings; SI units and symbols are used;
(ix) solvent ratios are given in volume:volume (v/v) terms; and
(x) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺;
(xi) unless stated otherwise compounds containing an asymmetrically substituted carbon and/or sulphur atom have not been resolved;
(xii) where a synthesis is described as being analogous to that described in a previous example the amounts used are the millimolar ratio equivalents to those used in the previous example;
(xvi) the following abbreviations have been used:
THF tetrahydrofuran;
DMF N,N-dimethylformamide;
EtOAc ethyl acetate;
MeOH methanol;
ether diethyl ether;
EtOH ethanol;
HATU O-(7-azabenzotriazol-1-yl)-1,1-3,3-tetramethyluronium hexafluorophosphate;
HBTU O-benzotriazol-1-yl-N,N,N′,N′-tetramethyluronium hexafluorophosphate;
DCM dichloromethane;
DMFDMA N,N-dimethylformamide dimethylacetal;
TEA triethylamine;
DIPEA diisopropylethylamine;
EDAC 1-ethyl-3-(3-dimethylaminopropyl)carbodiimide hydrochloride;
BINAP 2,2′-bis(diphenylphosphino)-1,1′-binaphthyl;
HPLC high pressure liquid chromatography;
RPHPLC reverse phase high pressure liquid chromatography;
MPLC medium pressure liquid chromatography;
DDQ 2,3-dichloro-5,6-dicyano-1,4-benzoquinone;
selectflour 1-chloromethyl-4-fluoro-1,4-diazoniabicyclo[2.2.2]octane bis(tetrafluoroborate);
DMSO dimethylsulphoxide; and
xantphos 9,9-dimethyl-4,5-Bis(diphenylphosphino)xanthene;
   xvii) where an Isolute SCX-2 column is referred to, this means an “ion exchange” extraction cartridge for adsorption of basic compounds, i.e. a polypropylene tube containing a benzenesulphonic acid based strong cation exchange sorbent, used according to the manufacturers instructions obtained from International Sorbent Technologies Limited, Dyffryn Business Park, Hengeod, Mid Glamorgan, UK, CF82 7RJ;
   xviii) macroporous polystyrene carbonate resin refers to, Argonaut Technologies MP carbonate resin with the capacity 3.0 Mole equivalents per gram of resin available from Argonaut Technologies, New Road, Hengoed, Mid Glamorgan United Kingdom, CF82 8AU;
   xix) Where a Biotage cartridge/column is referred to, this means a pre-packed chromatography cartridge for separation of compounds in a mixture, i.e. a polypropylene tube containing silica gel, used according to the manufacturers instructions obtained from Biotage UK Ltd., Harforde Court, Foxholes Business Park, John Tate Road, Hertford, SG13 7NW, United Kingdom; and
   xx) Acidic preparative HPLC refers to the use of a Phenomenex 150×21.2 mm Luna 10 micron C18 column using a gradient of 5 to 95 of 0.2% TFA water-acetonitrile over 10 mins at 20 ml/minute flow rate.

### Example 1

### 6-{[4-(1-Isopropyl-2-methyl-1H-imidazol-5-yl)pyrimidin-2-yl]amino}-N-methylnicotinamide

Ethyl 6-{[4-(1-isopropyl-2-methyl-1H-imidazol-5-yl)pyrimidin-2-yl]amino}nicotinate (Method 17; 100 mg, 0.28 mmol) and 33% methylamine/EtOH (4 ml) were heated at 150° C. for 2 h under microwave conditions. The solution was concentrated in vacuo, and the residue was partitioned between DCM and water and sat. sodium hydrogen carbonate and the aqueous layer was extracted with DCM twice. The organics were combined, washed with brine, dried and the solvent was evaporated to give the title compound as a solid which was dried in vac oven overnight at 50° C. (43 mg, 44%). NMR (400 MHz): 1.49 (d, 6H), 2.54 (s, 3H under DMSO signal), 2.80 (d, 3H), 5.93 (septet, 1H), 7.25 (d, 1H), 7.55 (s, 1H), 8.16 (dd, 2H), 8.22 (d, 2H), 8.43 (q, 2H), 8.50 (d, 1H), 8.77 (d, 1H), 10.17 (s, 1H); m/z 352.

### Example 2

### 6-{[4-(1-Isopropyl-2-methyl-1H-imidazol-5-yl)pyrimidin-2-yl]amino}-N,N-dimethylnicotinamide

A solution of 6-{[4-(1-isopropyl-2-methyl-1H-imidazol-5-yl)pyrimidin-2-yl]amino}nicotinic acid (Method 18; 133.7 mg, 0.4 mmol), 33% dimethylamine/EtOH (0.1 ml), HOBt.H₂O (73.5 mg, 0.48 mmol) and DIPEA (0.08 ml, 0.48 mmol) in DMF (4 ml) was cooled to 0° C., and EDAC (92 mg, 0.48 mmol) was added in portions. The mixture was stirred at ambient temperature for 3 hours, then at 90° C. for 2.5 days. The solution was concentrated in vacuo, and the residue was partitioned between DCM and water and sat. sodium hydrogen carbonate. The organic extract was washed with water (2 times), brine and dried. The residue obtained on evaporation was purified by chromatography eluting with MeOH:DCM (1:99 to 5:95) to yield the title compound, after trituration with ether, as a solid which was dried in vac oven overnight at 50° C. (16 mg, 11%). NMR (400 MHz): 1.47 (d, 6H), 2.51 (s, 3H), 2.97 (s, 6H), 5.89 (septet, 1H), 7.23 (d, 1H), 7.53 (s, 1H), 7.84 (dd, 1H), 8.19 (d, 1H), 8.39 (d, 1H), 8.49 (d, 1H), 10.10 (s, 1H), m/z 366.

### Example 3

### 6-{[5-Fluoro-4-(1-isopropyl-2-methyl-1H-imidazol-5-yl)pyrimidin-2-yl]amino}nicotinamide

2-Amino-5-fluoro-4-(1-isopropyl-2-methyl-1H-imidazol-5-yl)pyrimidine (Method 1; 517 mg, 2.2 mmol), 6-chloronicotinamide (313.14 mg, 2 mmol), tris(dibenzylideneacetone) dipalladium(0) (12.8 mg, 0.7 mol %), BINAP (13.7 mg, 1.1 mol %) and caesium carbonate (912.3 mg, 2.8 mmol) in anhydrous 1,4-dioxane (6 ml) were evacuated and refilled with nitrogen (3 times). The reaction was heated under nitrogen at 100° C. overnight. Extra tris(dibenzylideneacetone)dipalladium(0) (12.8 mg, 0.7 mol %) and BINAP (13.7 mg, 1.1 mol %) were added and the reaction mixture was heated under nitrogen at 100° C. for 4.5 h before evaporating under reduced pressure. The residue obtained was partitioned between DCM and water and the aqueous layer was extracted with DCM twice. The precipitate formed was filtered off to give a solid corresponding to the required product plus impurity. The organics were combined, washed with brine, dried and the solvent was evaporated to give a solid corresponding to unreacted SM. The residue filtered off was purified by reverse phase chromatography (acidic prep HPLC system). The product containing fractions were passed through a pre-equilibrated Isolute SCX-2 column, eluting with MeOH, then a 7 molar solution of ammonia in MeOH. Evaporation of solvent gave the title compound as a solid which was dried in vac oven overnight at 50° C. (60 mg, 15%). NMR (400 MHz): 1.48 (d, 6H), 2.56 (s, 3H), 5.71 (septet, 1H), 7.34 (br s, 1H), 7.49 (d, 1H), 7.97 (br s, 1H), 8.09 (d, 1H), 8.19 (dd, 1H), 8.66 (d, 1H), 8.80 (d, 1H), 10.34 (s, 1H); ¹⁷F NMR (376.461 MHz): −145.26 (t, 1F); m/z 356.

### Example 4

### 6-{[5-Fluoro-4-(1-isopropyl-2-methyl-1H-imidazol-5-yl)pyrimidin-2-yl]amino}-N-methylnicotinamide

Methyl 6-{[5-fluoro-4-(1-isopropyl-2-methyl-1H-imidazol-5-yl)pyrimidin-2-yl]amino}nicotinate (Method 22; 295.3 mg, 0.80 mmol) and 33% methylamine/EtOH (5 ml) were heated at 150° C. for 4 h under microwave conditions. The solution was concentrated in vacuo, then the residue was partitioned between DCM and water and sat. sodium hydrogen carbonate and the aqueous layer was extracted with DCM twice. The precipitate formed was filtered off to give a solid corresponding to the required product. The organics were combined, washed with brine and dried. The residue obtained on evaporation of solvent was combined with the solid filtered off from aqueous work up and purified by chromatography eluting with MeOH:DCM (1:99 to 5:95) to give a solid which was dried in vac oven overnight at 50° C. after evaporation of solvent to give the title compound (183 mg, 62%). NMR (400 MHz): 1.48 (d, 6H), 2.55 (s, 3H), 2.80 (d, 3H), 5.72 (septet, 1H), 7.47 (d, 1H), 8.10 (d, 2H), 8.16 (dd, 2H), 8.42 (q, 1H), 8.65 (d, 1H), 8.76 (d, 1H), 10.32 (s, 1H); ¹⁷F NMR (400 MHz): −145.34 (t, 1F); m/z 370.

### Example 5

### 6-{[5-Fluoro-4-(1-isopropyl-2-methyl-1H-imidazol-5-yl)pyrimidin-2-yl]amino}-N,N-dimethylnicotinamide

A solution of 6-{[5-fluoro-4-(1-isopropyl-2-methyl-1H-imidazol-5-yl)pyrimidin-2-yl]amino}nicotinic acid (Method 19; 341 mg, 0.96 mmol), HATU (1.09 g, 2.88 mmol) and DIPEA (0.24 ml, 1.44 mmol) in DMF (10 ml) was stirred at ambient temperature for 1 h, followed by addition of 33% dimethylamine/EtOH (0.51 ml, 2.88 mmol). The reaction mixture was stirred at ambient temperature for 4 h before evaporating under reduced pressure. The residue obtained was partitioned between DCM and water and sat. sodium hydrogen carbonate. The organic extract was washed with water (2 times), brine and dried. The residue obtained on evaporation was purified by chromatography eluting with MeOH:DCM (1:99 to 5:95) to give a solid which was dissolved in MeOH and passed through a pre-equilibrated Isolute SCX-2 column, eluting with MeOH, then a 7 molar solution of ammonia in MeOH. Evaporation of solvent gave the title compound as a solid which was dried in vac oven overnight at 50° C. (180 mg, 42%). NMR (400 MHz): 1.47 (d, 6H), 2.54 (s, 3H), 3.00 (s, 6H), 5.68 (septet, 1H), 7.47 (d, 1H), 7.84 (dd, 1H), 8.06 (d, 1H), 8.38 (d, 1H), 8.64 (d, 1H), 10.25 (s, 1H); ¹⁷F NMR (400 MHz): −145.68 (t, 1F); m/z 384.

### Examples 6 and 7

The following compounds were prepared using the procedure of Example 5 from 6-{[5-fluoro-4-(1-isopropyl-2-methyl-1H-imidazol-5-yl)pyrimidin-2-yl]amino}pyridazine-3-carboxylic acid (Method 20) and the appropriate amine.

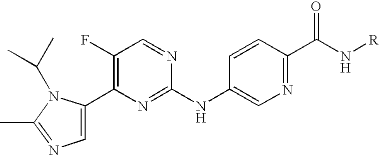

| Ex | R | NMR(400 MHz) | m/z |
|---|---|---|---|
| 6¹ | Cyclo- | 0.68(m, 4 H), 1.46(d, 6 H), 2.53(s, 3 H), | 396 |
| | propyl | 2.88(sextet, 1 H), 5.36(septet, 1 H), 7.40(d, 1 H), | |
| | | 7.96(d, 1 H), 8.33(dd, 1 H), 8.48(d, 1 H), | |
| | | 8.64(d, 1 H), 8.81(d, 1 H), 10.03(s, 1 H); | |
| | | ¹⁷F NMR: −146.53(t, 1 F) | |
| 7² | Et | 1.13(t, 3 H), 1.47(d, 6 H), 3.32(q, 2 H under water | 384 |
| | | signal), 5.38(septet, 1 H), 7.41(d, 1 H), 7.97(d, | |
| | | 1 H), 8.33(dd, 1 H), 8.56(t, 1 H), 8.65(d, 1 H), | |
| | | 8.83(d, 1 H), 10.03(s, 1 H); ¹⁷F | |
| | | NMR: −146.55(t, 1 F) | |

¹Purified by chromatography with MeOH:DCM (1:99 to 7:93) and reverse phase chromatography (acidic prep HPLC) and Isolute SCX-2 column (200 mg, 60%).
²Most crude product filtered off from aqueous work up. Purified by reverse phase chromatography (acidic prep HPLC system) and Isolute SCX-2 column (150 mg, 47%).

### Example 8

### 5-{[5-Fluoro-4-(1-isopropyl-2-methyl-1H-imidazol-5-yl)-pyrimidin-2-yl]amino}-N-methylpyridine-2-carboxamide

To a solution of 2M methylamine/THF (0.16 ml, 3.12 mmol) in THF (2 ml) was added 2M trimethyl aluminium/toluene (1.2 ml, 2.34 mmol) and the system was stirred at room temperature until no effervescence was observed. A solution of ethyl 5-{[5-fluoro-4-(1-isopropyl-2-methyl-1H-imidazol-5-yl)pyrimidin-2-yl]amino}pyridine-2-carboxylate (Method 23; 300 mg, 0.78 mmol) in THF (3 ml) was added dropwise and the reaction mixture was heated at 80° C. for 6 h, then at 110° C. for 2 h. The reaction mixture was diluted with DCM and MeOH and passed through a pre-equilibrated Isolute SCX-2 column. Elution with MeOH then a 7 molar solution of ammonia in MeOH recovered the product. After evaporation of the solvent the residue obtained was partitioned between DCM and water and the aqueous layer was extracted with DCM twice. The organics were combined, washed with brine, dried and the solvent was evaporated to give the title compound as a solid which was dried in vac oven overnight at 50° C. (102 mg, 35%). NMR (400 MHz): 1.46 (d, 6H), 2.53 (s, 3H), 2.82 (d, 3H), 5.38 (septet, 1H), 7.40 (d, 1H), 7.97 (d, 1H), 8.31 (dd, 1H), 8.53 (q, 1H), 8.64 (d, 1H), 8.83 (d, 1H), 10.03 (s, 1H); ¹⁷F NMR (400 MHz): −146.56 (t, 1F); m/z 370.

### Example 9

### 5-{[5-Fluoro-4-(1-isopropyl-2-methyl-1H-imidazol-5-yl)pyrimidin-2-yl]amino}pyridine-2-carboxamide

5-{[5-Fluoro-4-(1-isopropyl-2-methyl-1H-imidazol-5-yl)pyrimidin-2-yl]amino}pyridine-2-carbonitrile (Method 24; 287.8 mg, 0.85 mmol) and 2.5N aq NaOH (0.41 ml, 102 mmol) in THF/H₂O (6 ml/6 ml) were heated under reflux for 2 h. Extra 2.5N aq NaOH (0.1 ml, 0.26 mmol) added and the reaction was heated for 3 h under reflux before evaporating under reduced pressure. The reaction mixture was diluted with water and the resulting precipitate was filtered off and washed with water. The title compound was obtained as a solid that was dried in vac oven overnight at 50° C. (330 mg, 93%). NMR (400 MHz): 1.47 (d, 6H), 2.54 (s, 3H), 5.40 (septet, 1H), 7.40 (d, 1H), 7.42 (s, 1H), 7.90 (s, 1H), 7.98 (d, 1H), 8.30 (dd, 1H), 8.64 (d, 1H), 8.84 (d, 1H), 10.06 (s, 1H); ¹⁷F NMR (400 MHz): −146.60 (t, 1F); m/z 356.

### Example 10

### 6-{[4-(1-Isopropyl-2-methyl-1H-imidazol-5-yl)pyrimidin-2-yl]amino}-N,2-dimethylnicotinamide

The title compound was prepared from 6-{[4-(1-isopropyl-2-methyl-1H-imidazol-5-yl)pyrimidin-2-yl]amino}-2-methylnicotinic acid (Method 21; 370.53 mg, 1.05 mmol) and 33% methylamine/EtOH (0.2 ml, 4.87 mmol) by the procedure of Example 5. The residue obtained on evaporation of solvent was partitioned between DCM and water and sat. sodium hydrogen carbonate and the aqueous layer was extracted with DCM twice. The organics were combined, washed with brine, dried and concentrated. Purification was then performed by reverse phase chromatography (acidic prep HPLC system). The product containing fractions were passed through a pre-equilibrated Isolute SCX-2 column, eluted with MeOH, then a 7 molar solution of ammonia in MeOH. Evaporation of solvent gave the title compound as a solid that was dried in vac oven overnight at 50° C. (67.7 mg, 18%). NMR (400 MHz): 1.48 (d, 6H), 2.58 (s, 3H), 2.77 (d, 3H), 3.28 (d, 3H under water signal), 5.93 (septet, 1H), 7.22 (d, 1H), 7.54 (s, 1H), 7.74 (d, 1H), 8.09 (d, 1H), 8.18 (q, 1H), 8.47 (d, 1H), 9.90 (s, 1H); m/z 366.

### Example 11

### 4-(1-Isopropyl-2-methyl-1H-imidazol-5-yl)-N-[5-(methylthio)pyrazin-2-yl]pyrimidin-2-amine

Dry 1,4-dioxane (15 ml) was added to a mixture of 2-chloro-4-(1-isopropyl-2-methyl-1H-imidazol-5-yl)pyrimidine (Method 6; 501 mg, 2.11 mmol) and 5-(methylthio)pyrazin-2-amine (Method 4; 298 mg, 2.11 mmol) under nitrogen. Tris(dibenzylideneacetone) dipalladium(0) (298 mg), racemic-2,2-bis(diphenylphosphino)-1,1′-binaphthyl (70 mg) and sodium t-butoxide (243 mg, 2.53 mmol) were rapidly added sequentially. The reaction was stirred and heated at 84° C. under nitrogen for 20 hours. The solvent was removed in vacuo and the residue treated with water and ether and the suspension stirred for 30 minutes. The mixture was filtered and the filter washed with water and ether. The crude product was dried and triturated with EtOAc, filtered, washed with EtOAc and dried to give the title compound as a pale brown solid (460 mg, 64%). NMR: 1.43 (d, 6H), 2.48 (s, 3H+ DMSO), 2.53 (s, 3H), 5.82 (m, 1H), 7.2 (d, 2H), 7.5 (s, 1H), 8.32 (s, 1H), 8.43 (d, 1H), 9.17 (s, 1H), 10.11 (s, 1H); m/z 342.

### Example 12

### 4-(1-Isopropyl-2-methyl-1H-imidazol-5-yl)-N-[5-(methylsulfonyl)pyrazin-2-yl]pyrimidin-2-amine

To a stirred solution of 4-(1-isopropyl-2-methyl-1H-imidazol-5-yl)-N-[5-(methylthio)pyrazin-2-yl]pyrimidin-2-amine (Example 11; 150 mg, 0.439 mg) in acetic acid (2.0 ml), 35% hydrogen peroxide in water (0.3 ml) was added. The reaction was stirred and heated at 60° C. for 4 hours. Water was added followed by sodium metabisulphite and the mixture was stirred for 5 minutes. The pH of the mixture was adjusted to 10.5 with 40% sodium hydroxide solution and the mixture extracted with DCM. The organic layer was dried with anhydrous sodium sulphate, filtered and evaporated. The crude product was purified by flash chromatography eluting with MeOH:DCM (3:97) to give the title compound as a white solid (66 mg, 40%). NMR: 1.5 (d, 6H), 2.5 (s, 3H), 3.25 (s, 3H), 5.9 (m, 1H), 7.37 (d, 1H), 7.6 (s, 1H), 8.53 (d, 1H), 8.85 (s, 1H), 9.47 (s, 1H), 11.05 (s, 1H); m/z 374.

### Example 13

### 4-(1-Isopropyl-2-methyl-1H-imidazol-5-yl) -N-[5-(methylthiopyridin-2-yl]pyrimidin-2-amine

A mixture of N-[5-(methylthio)pyridin-2-yl]guanidine (Method 9; 304 mg, 1.67 mmol) and (2E)-3-(dimethylamino)-1-(1-isopropyl-2-methyl-1H-imidazol-5-yl)prop-2-en-1-one (Method 24 of WO 03/076436; 308 mg, 1.39 mmol) in dry 2-methoxyethanol (4.4 ml) was stirred and heated at reflux under nitrogen for 26 hours. The solvent was removed in vacuo and the residue treated with distilled water, filtered, washed with water and ether and dried to give the title compound as a pale yellow solid (333 mg. 70%). NMR: 1.45 (d, 6H), 2.7 (s, 6H+ DMSO), 5.83 (m, 1H), 7.13 (d, 1H), 7.47 (s, 1H), 7.73 (dd, 1H), 8.07 (d, 1H), 8.43 (d, 1H), 9.83 (s, 1H); m/z 341.

### Example 14

### 4-(1-Isopropyl-2-methyl-1H-imidazol-5-yl)-N-[5-(methylsulphonyl)pyridin-2-yl]pyrimidin-2-amine

To stirred suspension of 4-(1-isopropyl-2-methyl-1H-imidazol-5-yl)-N-[5-(methylthio)pyridin-2-yl]pyrimidin-2-amine (Example 13; 249 mg, 0.732 mmol) in MeOH (10 ml), acetone (2.5 ml) and water (1.25 ml), potassium peroxymonosulphate (585 mg, 0.952 mmol) was added. The mixture was stirred vigorously at room temperature for 5 hours. 10% Sodium bisulphite (3 ml) was added to the reaction mixture and it was stirred for 30 minutes. The mixture was concentrated in vacuo and water added. The pH of the suspension was adjusted to 7 with sodium bicarbonate solution and the mixture extracted with DCM. The organics were washed with water and saturated sodium chloride, dried, filtered and evaporated. The crude product was triturated with EtOAc, filtered, washed with EtOAc and dried to give the title compound as a white solid (187 mg, 69%). NMR: 1.5 (d, 6H), 2.53 (s, 3H+ DMSO), 3.25 (3, 3H), 5.9 (m, 1H), 7.3 (d, 1H), 7.57 (s, 1H), 8.2 (d, 1H), 8.35 (d, 1H), 8.52 (d, 1H), 8.75 (s, 1H), 10.59 (s, 1H); m/z 373.

### Example 15

### 4-(1-Isopropyl-2-methyl-1H-imidazol-5-yl)-N-[6-methyl-5-(methylthio)pyridin-2-yl]pyrimidin-2-amine

A mixture of N-[6-methyl-5-(methylthio)pyridin-2-yl]guanidine (Method 12; 224 mg, 1.14 mmol) and (2E)-3-(dimethylamino)-1-(1-isopropyl-2-methyl-1H-imidazol-5-yl)prop-2-en-1-one (Method 24 of WO 03/076436, 210 mg, 0.952 mmol) in dry 2-methoxyethanol (3.2 ml) was stirred and heated at reflux under nitrogen for 20 hours. The solvent was removed in vacuo, and the residue suspended in water and ether. The mixture was stirred 30 minutes, filtered washed with water and ether and dried to give the title compound as an off white solid (240 mg, 71%). NMR: 1.45 (d, 6H), 2.4 (s, 6H), 5.85 (m, 1H), 7.13 (d, 1H), 7.48 (s, 1H), 7.48 (s, 1H), 7.63 (d, 1H), 8.02 (d, 1H), 8.4 (d, 1H), 9.66 (s, 1H); m/z 355.

### Example 16

### 4-(1-Iso-propyl-2-methyl-1H-imidazol-5-yl)-N-[6-methyl-5-(methylsulphonyl)pyridin-2-yl]pyrimidin-2-amine

To stirred suspension of 4-(1-isopropyl-2-methyl-1H-imidazol-5-yl)-N-[6-methyl-5-(methylthio)pyridin-2-yl]pyrimidin-2-amine (Example 15; 178 mg, 0.502 mmol) in MeOH (7.2 ml), acetone (1.8 ml) and water (1.00 ml), potassium peroxymonosulphate (402 mg, 0.65 mmol) was added. The mixture was stirred vigorously at room temperature for 20 hours. 10% sodium bisulphite (3 ml) was added to the reaction mixture and it was stirred for 30 minutes. The mixture was concentrated in vacuo and water added. The pH of the suspension was adjusted to 7 with sodium bicarbonate solution and the mixture filtered and the filter washed with water and the solid dried. The crude product was purified by flash chromatography eluting with MeOH:DCM (2:98). The product off the column was triturated with ether/isohexane, filtered, washed with isohexane and dried to give the title compound as a white solid (39 mg, 20%). NMR: 1.47 (d, 6H), 2.5 (s, 3H+ DMSO), 2.73 (s, 3H), 3.23 (s, 3H), 5.9 (m, 1H), 7.28 (m, 1H), 7.38 (d, 1H), 7.55 (s, 1H), 8.13 (d, 1H), 8.37 (d, 1H), 8.5 (d, 1H), 10.42 (s, 1H); m/z 387.

### Example 17

### 5-Fluoro-4-(1-isopropyl-2-methyl-1H-imidazol-5-yl)-N-[5-(methylsulphonyl)pyridin-2-yl]pyrimidin-2-amine

By the procedure of Example 16,5-fluoro-4-(1-isopropyl-2-methyl-1H-imidazol-5-yl)-N-[5-(methylthio)pyridin-2-yl]pyrimidin-2-amine (Example 18; 169 mg, 0.472 mmol) and potassium peroxymonosulphate (379 mg, 408 mg) to give the title compound as a white solid (84 mg, 46%). NMR: 1.47 (d, 6H), 2.53 (s, 3H), 3.25 (s, 3H), 5.7 (m. 1H), 7.47 (d, 1H), 8.2 (m, 2H), 8.67 (d, 1H), 8.73 (s, 1H), 10.73 (s, 1H); m/z 391.

### Example 18

### 5-Fluoro-4-(1-isopropyl-2-methyl-1H-imidazol-5-yl)-N-[5-(methylthio)pyridin-2-yl]pyrimidin-2-amine

A mixture of N-[5-(methylthio)pyridin-2-yl]guanidine (Method 9; 300 mg, 1.64 mmol) and (2Z)-3-(dimethylamino)-2-fluoro-1-(1-isopropyl-2-methyl-1H-imidazol-5-yl)prop-2-en-1-one (Method 26; 328 mg, 1.37 mmol) in dry 2-methoxyethanol (4.4 ml) was stirred and heated at reflux under nitrogen for 20 hours. The solvent was removed in vacuo and the residue dissolved in DCM and water. The layers were partitioned and separated. The organic layer was washed in turn with water, saturated sodium bicarbonate, water, saturated sodium chloride and dried with anhydrous sodium sulphate, filtered and evaporated. The crude product was purified by flash chromatography eluting with MeOH:DCM (2:98). The product off the column was triturated with ether, filtered washed with ether and dried to give the title compound as a white solid (262 mg, 53%). NMR: 1.43 (d, 6H), 2.47 (s, 3H+ DMSO), 2.53 (s, 3H), 5.62 (m, 1H), 7.42, d, 1H), 7.73 (dd, 1H), 7.95 (d, 1H), 8.23 (d, 1H), 8.57 (d, 1H), 10.0 9 s, 1H); m/z 359.

### Example 19

### 5-Fluoro-4-(1-isopropyl-2-methyl-1H-imidazol-5-yl)-N-[6-methyl-5-(methylthio)pyridin-2-yl]pyrimidin-2-amine

By the procedure of Example 18, N-[6-methyl-5-(methylthio)pyridin-2-yl]guanidine (Method 12; 199 mg, 1.01 mmol) and (2Z)-3-(dimethylamino)-2-fluoro-1-(1-isopropyl-2-methyl-1H-imidazol-5-yl)prop-2-en-1-one (Method 26; 201 mg, 0.84 mmol) were reacted together to give the title compound as a yellow solid (172 mg, 55%). NMR: 1.45 (s, 6H), 2.43 (s, 3H), 2.45 (s, 3H), 2.53 (s, 3H, 5.62 (m, 1H), 7.43 (d, 1H), 7.63 (d, 1H), 7.93 (d, 1H), 8.53 (d, 1H), 9.83 (s, 1H); m/z 373.

### Example 20

### 5-Fluoro-4-(1-isopropyl-2-methyl-1H-imidazol-5-yl)-N-[6-methyl-5-(methylsulphonyl pyridin-2-yl]pyrimidin-2-amine

The title compound was prepared by the procedure of Example 16 using 5-fluoro-4-(1-isopropyl-2-methyl-1H-imidazol-5-yl)-N-[6-methyl-5-(methylthio)pyridin-2-yl]pyrimidin-2-amine (Example 19; 148 mg, 0.398 mmol) and potassium peroxymonosulphate (318 mg, 0.517 mmol). The crude product was purified by flash chromatography eluting with MeOH:DCM (2:98). The product off the column was triturated with ether-isohexane, filtered washed with isohexane and dried to give the title compound as white solid (90 mg, 56%). NMR: 1.48 (d, 6H), 2.55 (s, 3H), 2.73 (s, 3H), 3.23 (s, 3H), 5.65 (m, 1H), 7.47 (d, 1H), 8.13 (s, 2H), 8.65 (d, 1H), 10.58 (s, 1H); m/z 405.

### Example 21

### 5-Fluoro-4-(1-isopropyl-2-methyl-1H-imidazol-5-yl)-N-(6-morpholin-4-ylpyridin-3-yl)pyrimidin-2-amine

N-(6-Morpholin-4-ylpyridin-3-yl)guanidine hydrochloride salt (Method 13; 750 mg, 2.588 mmol, 1.2 eq) and (2Z)-3-(dimethylamino)-2-fluoro-1-(1-isopropyl-2-methyl-1H-imidazol-5-yl)prop-2-en-1-one (Method 26; 513 mg, 2.15 mmol, 1.0 eq) were combined in methoxyethanol (15 ml) and sodium methoxide solution (1.18 ml, 5.16 mmol, 2.4 eq) was added. The mixture was heated to 130° C. for 72 hours. The mixture was concentrated and chromatographed (Biotage 40 S, eluent 0-10% EtOH:EtOAc) and the product containing fractions were concentrated and purified further by prep HPLC (acidic system). The product containing fractions were pooled and adsorbed onto an Isolute SCX-2 cartridge then liberated with 1M ammonia in MeOH solution before concentrating to give the product (216 mg, 25%) as a pale yellow solid. NMR: 1.34 (d, 6H), 2.47 (s, 3H), 3.34 (t, 4H), 3.68 (t, 4H), 5.40 (m, 1H), 7.80 (d, 1H), 7.34 (d, 1H), 7.71 (dd, 1H), 8.27 (d, 1H), 8.43 (d, 1H), 9.19 (s, 1H); m/z 398.

### Example 22

### 4-(1-Isopropyl-2-methyl-1H-imidazol-5-yl)-N-(6-morpholin-4-ylpyridin-3-yl)pyrimidin-2-amine

N-(6-Morpholin-4-ylpyridin-3-yl)guanidine (Method 13; 750 mg, 2.58 mmol, 1.2 eq) and (2E)-3-(dimethylamino)-1-(1-isopropyl-2-methyl-1H-imidazol-5-yl)prop-2-en-1-one (Method 24 of WO 03/076436; 475 mg, 2.15 mmol, 1.0 eq) were combined in methoxyethanol (15 ml) and sodium methoxide solution (1.18 ml, 5.16 mmol, 2.4 eq) was added. The mixture was heated to 130° C. for 72 hours. After this time the mixture was concentrated and chromatographed (Biotage 40S, eluent 0-10% EtOH:EtOAc) and the product containing fractions were concentrated and purified further by prep HPLC (acidic system) The product containing fractions were pooled and adsorbed onto an Isolute SCX-2 cartridge then liberated with 1M ammonia in MeOH solution before concentrating to give the product (204 mg, 26%) as a pale yellow solid. NMR: 1.35 (d, 6H), 2.44 (s, 3H), 3.35 (t, 4H), 3.69 (t, 4H), 5.63 (m, 1H), 6.81 (d, 1H), 6.95 (d, 1H), 7.40 (s, 1H), 7.75 (dd, 1H), 8.29 (d, 1H), 8.30 (s, 1H), 9.13 (s, 1H); m/z 380.

### Example 23

### N-(5-Chloro-6-morpholin-4-ylpyridin-3-yl)-4-(1-isopropyl-2-methyl-1H-imidazol-5-yl)pyrimidin-2-amine

N-(5-Chloro-6-morpholin-4-ylpyridin-3-yl)guanidine bicarbonate salt (Method 16; 450 mg, 1.42 mmol, 1.1 eq) and (2E)-3-(dimethylamino)-1-(1-isopropyl-2-methyl-1H-imidazol-5-yl)prop-2-en-1-one (Method 24 of WO 03/076436; 285 mg, 1.29 mmol, 1.0 eq) were combined in methoxyethanol (15 ml) at room temperature, and the mixture was heated to 130° C. for 72 hours. After this time the mixture was concentrated, chromatographed (Biotage 40S, eluent 0-10% EtOH:EtOAc) and the product containing fractions concentrated and purified further by prep HPLC (acid prep). The product containing fractions were pooled and adsorbed onto an Isolute SCX-2 cartridge then liberated with 1M ammonia in MeOH solution before concentrating to give the product (208 g, 36%) as a pale yellow solid. NMR: 1.40 (d, 6H), 2.46 (s, 3H), 3.14 (t, 4H), 3.72 (t, 4H), 5.57 (m, 1H), 7.06 (d, 1H), 7.42 (s, 1H), 8.13 (d, 1H), 8.38 (d, 1H), 8.45 (d, 1H), 9.49 (s, 1H); m/z 414.

### Example 24

### N-(5-Chloro-6-morpholin-4-ylpyridin-3-yl)-5-fluoro-4-(1-isopropyl-2-methyl-1H-imidazol-5-yl)pyrimidin-2-amine

N-(5-Chloro-6-morpholin-4-ylpyridin-3-yl)guanidine bicarbonate salt (Method 16; 450 mg, 1.42 mmol, 1.1 eq) and (2Z)-3-(dimethylamino)-2-fluoro-1-(1-isopropyl-2-methyl-1H-imidazol-5-yl)prop-2-en-1-one (Method 26; 309 mg, 1.29 mmol, 1.0 eq) were combined in methoxyethanol (15 ml) at room temperature, and the mixture was heated to 130° C. for 72 hours. The mixture was concentrated and chromatographed (Biotage 40S, eluent 0-10% EtOH:EtOAc) the product containing fractions were concentrated and purified further by prep HPLC (acid prep). The product containing fractions were pooled and adsorbed onto an Isolute SCX-2 cartridge then liberated with 1M ammonia in MeOH solution before concentrating to give the product (208 g, 36%) as a pale yellow solid. NMR: 1.39 (d, 6H), 2.49 (s, 3H), 3.14 (t, 4H), 3.72 (t, 4H), 5.33 (m, 1H), 7.35 (d, 1H), 8.01 (d, 1H), 8.43 (d, 1H), 8.53 (d, 1H), 9.57 (s, 1H); m/z 432.

### Example 25

### 4-(1-Isopropyl-2-methyl-1H-imidazol-5-yl)-N-[6-(morpholin-4-ylcarbonyl)pyridin-3-yl]pyrimidin-2-amine

HBTU (0.22 g) was added to a stirred suspension of the lithium salt of 5-[4-(2-methyl-3-propan-2-yl-imidazol-4-yl)pyrimidin-2-yl]aminopyridine-2-carboxylic acid (Method 28; 0.20 g) in DMF (10 ml). After stirring for 20 mins at ambient temperature, morpholine (0.06 mL) was added followed by DIPEA (0.24 ml) and stirring was continued at ambient temperature for 24 hours. The reaction mixture was diluted with EtOAc (100 ml) and washed with 1N NaOH (100 ml), the aqueous solution extracted with EtOAc (100 ml) and the combined organic extracts were washed with brine. The organic layer was dried, filtered and evaporated to give a gum. Purification by RPHPLC gave the title compound as a colourless foam; ¹H NMR (400.132 MHz) 1.47 (d, 6H), 2.51 (s, 3H), 3.63 (m, 8H), 5.64 (m, 1H), 7.16 (d, 1H), 7.46 (s, 1H), 7.63 (d, 1H), 8.25 (d, 1H), 8.47 (d, 1H), 8.90 (s, 1H), 9.88 (bs, 1H); m/z 408.

### Examples 26 to 29

The following compounds were prepared using the procedure of Example 25 from the lithium salt of 5-[4-(2-methyl-3-propan-2-yl-imidazol-4-yl)pyrimidin-2-yl]aminopyridine-2-carboxylic acid (Method 28) and the appropriate amine.

| Ex | Compound | NMR (400.132 MHz) | m/z |
|---|---|---|---|
| 26 | (4-Methylpiperazin-1-yl)-[5- | 1.47 (d, 6H), 2.20 (s, 3H), | 421 |
| | [4-(2-methyl-3-propan-2-yl- | 2.34 (m, 4H), 2.51 (s, 3H) | |
| | 3H-imidazol-4-yl)pyrimidin-2- | 3.59 (m, 4H), 5.64 (m, 1H), | |
| | yl]aminopyridin-2-yl]- | 7.16 (d, 1H), 7.47 (s, 1H), | |
| | methanone | 7.58 (d, 1H), 8.22 (d, 1H), | |
| | | 8.47 (d, 1H), 8.88 (s, 1H), | |
| | | 9.85 (bs, 1H) | |
| 27 | (4-Methyl-1,4-diazepan-1-yl)- | 1.47 (d, 6H), 1.83 (m, 2H), | 435 |
| | [5-[4-(2-methyl-3-propan-2-yl- | 2.27 (m, 3H), 2.51 (s, 3H), | |
| | 3H-imidazol-4-yl)pyrimidin-2- | 2.55 (m, 3H), 2.63 (m, 1H), | |
| | yl]aminopyridin-2-yl]- | 3.59 (m, 4H), 5.65 (m, 1H), | |
| | methanone | 7.15 (d, 1H), 7.46 (s, 1H), | |
| | | 7.56 (m, 1H), 8.20 (d, 1H), | |
| | | 8.47 (d, 1H), 8.88 (s, 1H), | |
| | | 9.83 (bs, 1H) | |
| 28 | 5-{[4-(1-Isopropyl-2-methyl- | 1.48 (d, 6H), 1.69 (m, 4H), | 435 |
| | 1H-imidazol-5-yl)pyrimidin-2- | 1.99 (m, 2H), 2.17 (s, 3H), | |
| | yl]amino}-N-(1- | 2.51 (s, 3H), 2.73 (m, 2H), | |
| | methylpiperidin-4-yl)pyridine- | 3.75 (m, 1H), 5.63 (m, 1H), | |
| | 2-carboxamide | 7.18 (d, 1H), 7.48 (s, 1H), | |
| | | 7.97 (d, 1H), 8.28 (d, 1H), | |
| | | 8.41 (m, 1H), 8.48 (d, 1H), | |
| | | 8.87 (s, 1H), 9.94 (bs, 1H) | |
| 29 | N-(6-{[(3S)-3- | 1.48 (m, 6H), 1.72 (m, 1H), | 435 |
| | (Dimethylamino)pyrrolidin-1- | 2.06 (m, 1H), 2.17 (m, 6H), | |
| | yl]carbonyl}pyridin-3-yl)-4-(1- | 2.51 (s, 3H), 2.69 (m, 1H), | |
| | isopropyl-2-methyl-1H- | 3.50 (m, 1H), 3.72 (m, 2H), | |
| | imidazol-5-yl)pyrimidin-2- | 3.93 (m, 1H), 5.66 (m, 1H), | |
| | amine | 7.17 (m, 1H), 7.47 (s, 1H), | |
| | | 7.76 (d, 1H), 8.23 (m, 1H), | |
| | | 8.48 (d, 1H), 8.94 (d, 1H), | |
| | | 9.90 (bs, 1H) | |

### Examples 30 to 33

The following compounds were prepared using the procedure of Method 27 using 2-amino-5-chloro-4-(1-isopropyl-2-methyl-1H-imidazol-5-yl)pyrimidine (Method 5 in WO05/075461) in place of 2-amino-4-(1-isopropyl-2-methyl-1H-imidazol-5-yl)pyrimidine and the bromopyridylamide shown in place of methyl-5-bromopyridine-2-carboxylate.

| Ex | Compound | NMR (400.132 MHz) | m/z | SM |
|---|---|---|---|---|
| 30 | 5-{[5-Chloro-4-(1- | 1.40 (d, 6H), 1.68 (m, 4H), | 469 | Method |
| | isopropyl-2-methyl- | 1.98 (m, 2H), 2.17 (s, 3H), | | 30 |
| | 1H-imidazol-5-yl) | 2.73 (m, 2H), 3.74 (m, 1H), | | |
| | pyrimidin-2-yl] | 4.78 (m, 1H), 7.28 (s, 1H), | | |
| | amino}-N-(1-methyl- | 7.97 (d, 1H), 8.31 (m, 2H), | | |
| | piperidin-4-yl) | 8.71 (m, 1H), 8.89 (m, 1H), | | |
| | pyridine-2- | 10.26 (s, 1H) | | |
| | carboxamide | | | |
| 31 | [5-[5-Chloro-4-(2- | 1.39 (d, 6H), 2.20 (s, 3H), | 455 | Method |
| | methyl-3-propan-2-yl- | 2.29 (m, 2H), 2.37 (m, 2H), | | 31 |
| | 3H-imidazol-4-yl)- | 3.51 (m, 2H), 3.63 (m, 2H), | | |
| | pyrimidin-2- | 4.80 (m, 1H), 7.29 (s, 1H), | | |
| | yl]aminopyridin-2-yl]- | 7.57 (d, 1H), 8.19 (m, 1H), | | |
| | (4-methylpiperazin-1- | 8.69 (m, 1H), 8.86 (m, 1H), | | |
| | yl)-methanone | 10.16 (s, 1H) | | |
| 32 | [5-[5-Chloro-4-(2- | 1.40 (d, 6H), 1.77 (m, 1H), | 469 | Method |
| | methyl-3-propan-2-yl- | 1.87 (m, 1H), 2.27 (m, 3H), | | 32 |
| | 3H-imidazol-4-yl)- | 2.55 (m, 2H), 2.63 (m, 2H), | | |
| | pyrimidin-2- | 3.52 (m, 2H), 3.62 (m, 2H), | | |
| | yl]aminopyridin-2-yl]- | 4.81 (m, 1H), 7.29 (s, 1H), | | |
| | (4-methyl-1,4- | 7.55 (m, 1H), 8.17 (m, 1H), | | |
| | diazepan-1-yl)- | 8.69 (m, 1H), 8.85 (m, 1H), | | |
| | methanone | 10.14 (m, 1H) | | |
| 33 | 5-Chloro-N-(6-{[(3S)- | 1.40 (d, 6H), 1.71 (m, 1H), | 469 | Method |
| | 3-(dimethylamino) | 2.05 (m, 1H), 2.14 (s, 3H), | | 33 |
| | pyrrolidin-1-yl] | 2.19 (s, 3H), 2.68 (m, 1H), | | |
| | carbonyl}pyridin-3- | 3.47 (m, 1H), 3.71 (m, 2H), | | |
| | yl)-4-(1-isopropyl- | 3.89 (m, 1H), 4.82 (m, 1H), | | |
| | 2-methyl-1H-imidazol- | 7.29 (d, 1H), 7.75 (d, 1H), | | |
| | 5-yl)pyrimidin-2- | 8.19 (m, 1H), 8.70 (s, 1H), | | |
| | amine | 8.90 (m, 1H), 10.20 (s, 1H) | | |

### Examples 34 to 37

The following compounds were prepared using the procedure of Method 27 using 4-(2-cyclopropyl-3-isopropyl-3H-imidazol-4-yl)-pyrimidin-2-ylamine (Method 37) in place of 2-amino-4-(1-isopropyl-2-methyl-1H-imidazol-5-yl)pyrimidine and the bromopyridyl amide shown in place of methyl-5-bromopyridine-2-carboxylate.

| Ex | Compound | NMR (400.132 MHz) | m/z | SM |
|---|---|---|---|---|
| 34 | 5-{[4-(2-Cyclopropyl- | 1.00 (m, 4H), 1.59 (d, 6H), | 461 | Method |
| | 1-isopropyl-1H- | 1.71 (m, 4H), 1.99 (m, 2H), | | 30 |
| | imidazol-5-yl) | 2.17 (m, 4H), 2.73 (m, 2H), | | |
| | pyrimidin-2-yl] | 3.75 (m, 1H), 5.73 (m, 1H), | | |
| | amino}-N-(1-methyl- | 7.16 (d, 1H), 7.43 (s, 1H), | | |
| | piperidin-4-yl) | 7.98 (d, 1H), 8.29 (d, 1H), | | |
| | pyridine-2-carbox- | 8.41 (m, 1H), 8.47 (d, 1H), | | |
| | amide | 8.87 (d, 1H), 9.94 (s, 1H) | | |
| 35 | [5-[4-(2-Cyclopropyl- | 1.00 (m, 4H), 1.58 (d, 6H), | 447 | Method |
| | 3-propan-2-yl-3H- | 2.14-2.23 (m, 4H), 2.34 (m, | | 31 |
| | imidazol-4-yl) | 4H), 3.59 (m, 4H), 5.74 (m, | | |
| | pyrimidin-2-yl] | 1H), 7.14 (d, 1H), 7.42 (s, | | |
| | aminopyridin-2-yl]- | 1H), 7.59 (d, 1H), 8.23 (m, | | |
| | (4-methylpiperazin- | 1H), 8.45 (d, 1H), 8.88 (d, | | |
| | 1-yl)-methanone | 1H), 9.85 (s, 1H) | | |
| 36 | [5-[4-(2-Cyclopropyl- | 1.00 (m, 4H), 1.58 (m, 6H), | 461 | Method |
| | 3-propan-2-yl-3H- | 1.75-1.91 (m, 2H), 2.17 (m, | | 32 |
| | imidazol-4-yl) | 1H), 2.27 (m, 3H), 2.53- | | |
| | pyrimidin-2-yl] | 2.65 (m, 4H), 3.52-3.66 (m, | | |
| | aminopyridin-2-yl]-(4- | 4H), 5.74 (m, 1H), 7.14 (d, | | |
| | methyl-1,4-diazepan- | 1H), 7.42 (s, 1H), 7.56 (m, | | |
| | 1-yl)-methanone | 1H), 8.21 (m, 1H), 8.46 (m, | | |
| | | 1H), 8.88 (s, 1H), 9.83 (m, | | |
| | | 1H) | | |
| 37 | 4-(2-Cyclopropyl-1- | 1.00 (m, 4H), 1.59 (d, 6H), | 461 | Method |
| | isopropyl-1H- | 1.72 (m, 1H), 2.06 (m, 1H), | | 33 |
| | imidazol-5-yl)-N-(6-{ | 2.15-2.21 (m, 7H), 2.71 (m, | | |
| | [(3S)-3-(dimethyl- | 1H), 3.38-3.78 (m, 3H), | | |
| | amino)pyrrolidin-1-yl] | 3.93 (m, 1H), 5.75 (m, 1H), | | |
| | carbonyl}pyridin-3-yl) | 7.15 (m, 1H), 7.42 (s, 1H), | | |
| | pyrimidin-2-amine | 7.77 (m, 1H), 8.23 (m, 1H), | | |
| | | 8.47 (m, 1H), 8.94 (m, 1H), | | |
| | | 9.90 (m, 1H) | | |

### Example 38

### (2S)-1-[4-(6-{[4-(1-Cyclopentyl-2-methyl-1H-imidazol-5-yl)-5-fluoropyrimidine-2-yl]amino}pyridine-3-yl)piperazine-1-yl]-1-oxopropan-2-ol

4-(1-Cyclopentyl-2-methyl-1H-imidazol-5-yl)-5-fluoro-N-(5-piperazin-1-ylpyridine-2-yl)pyrimidine-2-amine (Example 52; 43 mg, 0.1 mmol), L-lactic acid (9 mg, 0.1 mmol), HATU (50 mg, 0.13 mmol), DIEA (50 μL, 42 mg, 0.29 mmol) and DMF (2 ml) were stirred at room temperature under nitrogen overnight. The DMF was removed. The product was purified by silica gel chromatography (5% MeOH/1% TEA/DCM), followed by semi Prep. HPLC. Gilson semi-prep. HPLC (25 mg, 35%). NMR (400 MHz): 1.21 (d, 3H), 1.56 (m, 2H), 1.76 (m, 2H), 1.96 (m, 2H), 2.20 (m, 2H), 2.79 (s, 3H), 3.20 (m, 4H), 3.75 (m, 4H), 4.49 (m, 1H), 5.39 (m, 1H), 7.88 (m, 2H), 8.00 (m, 1H), 8.19 (s, 1H), 8.89 (s, 1H); m/z 494.

### Examples 39-41

Following the method used to prepare Example 38 and substituting D-lactic acid, glycolic acid, and acetic anhydride, respectively, for L-lactic acid, the following analogues were prepared.

| Ex | Compound | NMR (400 MHz) | SM | mz |
|---|---|---|---|---|
| 39 | (2R)-1-[4-(6-{[4-(1- | 1.21 (d, 3H), 1.56 (m, 2H), | Example 52 + | 494 |
| | Cyclopentyl-2-methyl- | 1.76 (m, 2H), 1.96 (m, 2H), | D-lactic acid + | |
| | 1H-imidazol-5-yl)-5- | 2.20 (m, 2H), 2.79 (s, 3H), | HATU + | |
| | fluoropyrimidine-2- | 3.20 (m, 4H), 3.75 (m, 4H), | DIEA | |
| | yl]amino}pyridine-3- | 4.49 (m, 1H), 5.39 (m, 1H), | | |
| | yl)piperazine-1-yl]-1- | 7.88 (m, 2H), 8.00 (m, 1H), | | |
| | oxopropan-2-ol | 8.19 (s, 1H), 8.89 (s, 1H) | | |
| 40 | 2-[4-(6-{[4-(1- | 1.56 (m, 2H), 1.76 (m, 2H), | Example 52 + | 480 |
| | Cyclopentyl-2-methyl- | 1.99 (m, 2H), 2.20 (m, 2H), | glycolic | |
| | 1H-imidazol-5-yl)-5- | 2.75 (s, 3H), 3.19 (m, 4H), | acid + | |
| | fluoropyrimidin-2- | 3.54 (m, 2H), 3.65 (m, 2H), | HATU + | |
| | yl]amino}pyridin-3- | 4.15 (s, 2H), 5.39 (m, 1H), | DIEA | |
| | yl)piperazin-1-yl]-2- | 7.86 (m, 2H), 8.01 (m, 1H), | | |
| | oxoethanol | 8.19 (s, 1H), 8.89 (s, 1H) | | |
| 41 | N-[5-(4- | 1.70 (m, 2H), 1.89 (m, 2H), | Example 52 + | 464 |
| | Acetylpiperazin-1- | 2.04 (m, 2H), 2.09 (s, 3H), | acetic | |
| | yl)pyridin-2-yl]-4-(1- | 2.31 (m, 2H), 2.79 (s, 3H), | anhydride + | |
| | cyclopentyl-2-methyl- | 3.28 (m, 4H), 3.70 (m, 4H), | 5 mol % 4- | |
| | 1H-imidazol-5-yl)-5- | 4.49 (m, 1H), 5.45 (m, 1H), | dimethylamino- | |
| | fluoropyrimidin-2- | 7.50 (dd, 1H), 7.76 (m 1H), | pyridine + | |
| | amine | 8.04 (m, 1H), 8.19 (dd, | DIEA | |
| | | 1H), 8.82 (m, 1H) | | |

### Example 42

### [4-(3-Isopropyl-2-methyl-3H-imidazol-4-yl)-pyrimidin-2-yl]-(5-piperazin-1-yl-pyridin-2-yl)-amine

3-Dimethylamino-1-(3-isopropyl-2-methyl-3H-imidazol-4-yl)-propenone (Method 43; 0.44 g; 0.001 mol), N-(5-piperazin-1-yl-pyridin-2-yl)-guanidine (Method 45; 0.22 g; 0.001 mol), and potassium carbonate (−325 mesh, 0.52 g; 0.004 mol) in a 5 ml pressure tube, along with methoxyethanol (ca 3 ml). The tube was sealed and heated to 200° C. for 30 min, providing a brownish solution with suspended solids. This was filtered hot, and the filter cake was washed with more hot methoxyethanol. Removal of solvent under reduced pressure provided an amber semi-solid. This was purified by silica gel chromatography (10->30% EtOH in DCM; EtOH contained 5% v:v concentrated ammonium hydroxide). The most polar of the three major fractions obtained had an LC/MS displaying the desired ion. This fraction was further purified using reversed-phased preparative HPLC (3→14% acetonitrile) providing good separation from the impurities. Removal of solvent under reduced pressure provided the product as a gum. This material was subjected to lyophilization, to obtain the desired product as a glassy solid. NMR: 1.52 (d, 6H), 2.08 (s, 1H), 2.78 (s, 3H), 3.28 (b m, 4H), 3.36 (m, 4H), 5.81 (m, 1H), 7.29 (d, 1H), 7.65 (dd, 1H), 7.90 (d, 1H), 8.08 (d, 1H), 8.27 (s, 1H), 8.67 (d, 1H), 8.98 (br s, 2H), 10.43 (br s, 1H); m/z 379.

### Example 43

### [4-(3-Cyclopentyl-2-methyl-3H-imidazol-4-yl)-pyrimidin-2-yl]-(5-piperazin-1-yl-pyridin-2-yl)-amine

The title compound was prepared from N-(5-piperazin-1-yl-pyridin-2-yl)-guanidine (Method 45) and 3-dimethylamino-1-(3-cyclopentyl-2-methyl-3H-imidazol-4-yl)-propenone (Method 44) by the procedure of Example 42. NMR: 1.58 (m, 2H), 1.78 (m, 2H), 2.00 (m, 2H), 2.18 (m, 2H), 3.28 (br s, 4H), 3.35 (m, 4H), 5.76 (m, 1H), 7.23 (d, 1H), 7.57 (dd, 1H), 7.90 (d, 1H), 8.08 (d, 1H), 8.21 (s, 1H), 8.65 (d, 1H), 8.90 (br s, 1H), 10.25 (s, 1H) m/z 405.

### Example 44

### (R)-2-Hydroxy-1-(4-{6-[4-(3-isopropyl-2-methyl-3H-imidazol-4-yl)-pyrimidin-2-ylamino]-pyridin-3-yl}-piperazin-1-yl)-propan-1-one

A stock solution of [4-(3-isopropyl-2-methyl-3H-imidazol-4-yl)-pyrimidin-2-yl]-(5-piperazin-1-yl-pyridin-2-yl)-amine (Example 42) was prepared by dissolving 0.65 g of the material in 13 ml DMF, such that 1 ml of the stock solution contained 0.065 g (0.00017 mol). To 1.3 ml of the DMF solution (1.3 ml=0.065 g; 0.00017 mol) in a 5 ml pressure tube was added DIEA (0.04 g; 0.002 mol), followed by the D-lactic acid (0.02 g, 0.0002 mol), and then solid HATU (0.08 g; 0.002 mol) and another 1 ml DMF. The reaction mixture was stirred for 16 h. The solvent was removed under reduced pressure, and then the residue was partitioned between EtOAc and water. The aqueous phase was extracted twice with EtOAc, and then the combined organic layer was washed with water, then brine, and then dried. The solvent was removed under reduced pressure and the resulting residue was preabsorbed onto silica. Biotage purification (10-30% EtOH in DCM; EtOH “spiked” with 5% conc NH₄OH) provided the pure compound. NMR: 1.21 (d, 3H), 1.52 (d, 6H), 2.77 (s, 3.18 (m, 4H), 4.48 (m, 1H), 5.75 (m, 1H), 7.31 (d, 1H), 7.72 (m, 1H), 7.74 (m, 1H), 7.83 (d, 1H), 8.02 (d, 1H), 8.27 (s, 1H), 8.68 (d, 1H), 10.54 (br s, 1H); m/z 451.

### Examples 45-50

Following the method used to prepare Example 44 and substituting L-lactic acid, glycolic acid, and acetic anhydride, respectively, for D-lactic acid, the following analogues were prepared.

| Ex | Compound | NMR | SM | mz |
|---|---|---|---|---|
| 45 | (S)-2-Hydroxy-1- | 1.27 (d, 3H), 1.49 (d, | Example 42 + | 451 |
| | (4-{6-[4-(3-iso- | 6H), 3.19 (br m, 4H), | L-lactic | |
| | propyl-2-methyl- | 3.76 (br m, 4H), 4.54 | acid + | |
| | 3H-imidazol-4- | (m, 1H), 5.06 (d, 1H), | HATU + | |
| | yl)-pyrimidin-2- | 5.91 (m, 1H), 7.16 (d, | DIEA | |
| | ylamino]-pyridin- | 1H), 7.52 (dd, 1H), 7.55 | | |
| | 3-yl}-piperazin- | (s, 1H), 8.00 (d, 1H), | | |
| | 1-yl)-propan-1- | 8.11 (d, 1H), 8.45 (d, | | |
| | one | 1H), 9.63 (s, 1H) | | |
| 46 | 2-Hydroxy-1- | 1.51 (d, 6H), 2.76 (s, | Example 42 + | 437 |
| | (4-{6-[4-(3-iso- | 3H), 3.15 (br s, 4H), | glycolic | |
| | propyl-2-methyl- | 4.15 (s, 2H), 5.79 (m, | acid + | |
| | 3H-imidazol-4- | 1H), 7.25 (d, 1H), 7.59 | HATU + | |
| | yl)-pyrimidin-2- | (br d, 1H), 7.83 (d, 1H), | DIEA | |
| | ylamino]-pyridin- | 8.04 (d, 1H), 8.24 (s, | | |
| | 3-yl}-piperazin- | 1H), 8.65 (d, 1H) | | |
| | 1-yl)-ethanone | | | |
| 47 | 1-(4-{6-[4-(3- | 1.53 (d, 6H), 2.05 (s, | Example 42 + | 420 |
| | Isopropyl-2- | 3H), 2.78 (s, 3H), 3.17 | acetic | |
| | methyl-3H- | (m, 4H), 3.61 (m, 4H), | anhydride + | |
| | imidazol-4-yl)- | 5.72 (m, 1H), 7.35 (d, | 5 mol % 4- | |
| | pyrimidin-2-yl- | 1H), 7.81 (s, 2H), 8.01 | dimethylamino- | |
| | amino]-pyridin-3- | (m, 1H), 8.29 (s, 1H), | pyridine + | |
| | yl}-piperazin-1- | 8.71 (d, 1H), 10.75 (br | DIEA | |
| | yl)-ethanone | s, 1H) | | |
| 48 | (S)-2-Hydroxy-1- | 1.2 (t, 4H), 1.3 (t, 3H), | Example 43 + | 494 |
| | (4-{6-[4-(3- | 1.4 (t, 3H), 1.6 (s, 3H), | L-lactic | |
| | cyclopentyl-2- | 1.9 (m, 6H), 2.5 (s, 3H), | acid + | |
| | methyl-3H- | 3.2 (brS< 4H), 3.6 | HATU + | |
| | imidazol-4-yl)- | (brm, 4H) | DIEA | |
| | pyrimidin-2-yl- | | | |
| | amino]-pyridin-3- | | | |
| | yl}-piperazin- | | | |
| | 1-yl)-propan-1- | | | |
| | one | | | |
| 49 | (R)-2-Hydroxy-1- | 1.2 (d, 3H), 1.6 (brs, | Example 43 + | 494 |
| | (4-{6-[4-(3- | 2H), 1.9 (brs, 8H), 2.5 | D-lactic | |
| | cyclopentyl-2- | (S, 3H), 3.0 (m, 4H), | acid + | |
| | methyl-3H- | 3.5 (s, 1H), 3.7 (brs, | HATU + | |
| | imidazol-4-yl)- | 1H), 3.8 (brs, 1H), 4.4 | DIEA | |
| | pyrimidin-2- | (m, 1H), 5.5 (m, 1H), | | |
| | ylamino]-pyridin- | 6.9 (d, 1H), 7.2 (m, 1H), | | |
| | 3-yl}-piperazin- | 7.85 (s, 1H), 8.0 (s, 1H), | | |
| | 1-yl)-propan-1- | 8.3 (d, 1H), 8.4 (d, 1H) | | |
| | one | | | |
| 50 | 2-Hydroxy-1-(4- | 1.4 (d, 2H), 1.6 (m, 2H), | Example 43 + | 437 |
| | {6-[4-(3-cyclo- | 2.14 (m, 4H), 2.5 (s, | glycolic | |
| | pentyl-2-methyl- | 3H), 3.2 (brs, 4H), 3.6 | acid + | |
| | 3H-imidazol-4- | (brs, 2H), 3.8 (brs, 2H), | HATU + | |
| | yl)-pyrimidin-2- | 5.7 (m, 1H), 7.1 (s, 1H), | ethyldiisopro- | |
| | ylamino]-pyridin- | 7.5 (s, 1H), 7.6 (d, 1H), | opylamine | |
| | 3-yl}-piperazin- | 7.9 (s, 1H), 8.1 (s, 1H), | | |
| | 1-yl)-ethanone | 8.51 (s, 1H) | | |

### Example 51

### 4-(1-Cyclopentyl-2-methyl-1H-imidazol-5-yl)-5-Fluoro-N-(5-morpholin-4-ylpyridine-2-yl)pyrimidine-2-amine

N-(5-Morpholino-4-yl-pyridin-2-yl)guanidine hydrochloride, (Method 46; 33 mg, 0.1 mmol), K₂CO₃ (42 mg, 0.3 mmol), and (2Z)-1-(1-cyclopentyl-2-methyl-1H-imidazol-5-yl)-3-(dimethylamino)-2-fluoroprop-2-en-1-one (Method 47; 27 mg, 0.1 mmol) in methoxyethanol (2 ml) was heated at 125° C. overnight. The product was purified by Gilson semi-prep. HPLC (20 mg, 31%). NMR (400 MHz): 1.58 (m, 2H), 1.77 (m, 2H), 1.99 (m, 2H), 2.22 (m, 2H), 2.80 (s, 3H), 3.17 (m, 4H), 3.77 (m, 4H), 5.36 (m, 1H), 7.89 (d, 1H), 7.97 (m, 2H), 8.20 (m, 1H), 8.92 (m, 1H); m/z 423.

### Example 52

### 4-(1-Cyclopentyl-2-methyl-1H-imidazol-5-yl)-5-fluoro-N-(5-piperazin-1-ylpyridine-2-yl)pyrimidine-2-amine

N-(5-piperazine-1-ylpyridine-2-yl)guanidine hydrochloride (Method 45; 34 mg, 0.1 mmol), K₂CO₃ (42 mg, 0.3 mmol), and (2Z)-1-(1-cyclopentyl-2-methyl-1H-imidazol-5-yl)-3-(dimethylamino)-2-fluoroprop-2-en-1-one (Method 47; 27 mg, 0.1 mmol) in methoxyethanol (2 ml) was heated at 125° C. overnight. The product was purified by Gilson semi-prep. HPLC (19 mg, 25%). NMR (400 MHz): 1.59 (m, 2H), 1.76 (m, 2H), 2.01 (m, 2H), 2.21 (m, 2H), 2.80 (s, 3H), 3.24 (m, 4H), 3.42 (m, 4H), 5.39 (m, 1H), 7.82 (m, 1H), 7.94 (m, 2H), 8.05 (s, 1H), 8.18 (s, 1H), 8.89 (s, 1H); m/z 422.

### Preparation of Starting Materials

### Method 1

### 2-Amino-5-fluoro-4-(1-isopropyl-2-methyl-1H-imidazol-5-yl)pyrimidine

(2Z)-3-(Dimethylamino)-2-fluoro-1-(1-isopropyl-2-methyl-1H-imidazol-5-yl)prop-2-en-1-one (Method 26; 5 g, 20.90 mmol) and guanidine carbonate (7.53 g, 41.8 mmol) were heated at 135° C. overnight. Extra guanidine carbonate added (7.53 g, 41.8 mmol) and the mixture was heated at 135° C. for 3 h. The excess guanidine carbonate was filtered off and evaporation of the solvent gave a solid which was partitioned between EtOAc and water. The aqueous layer was extracted with EtOAc twice. The precipitate formed was filtered off to give pure product. The organics were combined, washed with brine, dried and concentrated to give a solid which was purified by chromatography eluting with MeOH:DCM:EtOAc (1:49.5:49.5 to 9:45.5:45.5). The title compound was obtained as a solid which was dried in vac oven overnight at 50° C. (3.3 g, 67%). NMR (400 MHz): 1.47 (d, 6H), 2.49 (s, 3H), 5.27 (septet, 1H), 6.57 (s, 2H), 7.28 (d, 1H), 8.28 (d, 1H); ¹⁷F NMR (400 MHz): −153.14 (t, 1F); m/z 236.

### Method 2

### Ethyl 2-methyl-6-oxo-1,6-dihydropyridine-3-carboxylate

Ethyl 2-methyl-6-oxo-1,4,5,6-tetrahydropyridine-3-carboxylate (2 g, 10.93 mmol) and DDQ (5.21 g, 22.95 mmol) in 1,4-dioxaxne (10 ml) were heated under reflux for 5 h. The residue obtained after evaporation of solvent under reduced pressure was partitioned between DCM and water and washed with sat. sodium hydrogen carbonate solution. The aqueous layer was extracted with DCM twice. The organics were combined, washed with brine, dried and the solvent was evaporated to give a solid which was purified by chromatography eluting with MeOH:DCM (1:99 to 4:96). Insoluble material in DCM and MeOH, prior to purification, was filtered off to give pure product. The product obtained after purification was dissolved in DCM and passed through a pre-equilibrated neutral alumina column, eluting with DCM, then 5% MeOH:DCM. Re-evaporation of solvent gave the title compound as a solid which was dried in vac oven overnight at 50° C. (1.23 g, 62%). NMR (400 MHz): 1.28 (t, 3H), 2.53 (s, 3H), 4.21 (q, 2H), 6.21 (d, 1H), 7.82 (d, 1H), 12.00 (s, 1H); m/z 182.

As an alternative to the above, the reaction mixture may be diluted with DCM and passed through a pre-equilibrated neutral alumina column, eluting with DCM, then MeOH. Chromatography on silica gel, then neutral alumina column eluting with DCM gave the product.

### Method 3

### Ethyl 6-chloro-2-methylnicotinate

Ethyl 2-methyl-6-oxo-1,6-dihydropyridine-3-carboxylate (Method 2; 2.281 g, 12.60 mmol) and phosphorus oxychloride (50 ml) were heated under reflux overnight. The oil obtained on evaporation of solvent was poured onto ice and solution neutralised with aq NH₃. The resulting precipitate was filtered off, washed with water and air-dried, to give the title compound as a solid (2.32 g, 81%). NMR (400 MHz): 1.33 (t, 3H), 2.70 (s, 3H), 4.33 (q, 2H), 7.49 (d, 1H), 8.21 (d, 1H); m/z 200-202.

### Method 4

### 5-(Methylthio)pyrazin-2-amine

A mixture of 5-bromopyrazin-2-amine (2.0 g, 11.56 mmol) and sodium thiomethoxide (1.62 g, 23.12 mmol) in dry dimethyl formamide (29 ml) was stirred and heated at 100° C. under nitrogen for 20 hours. The solvent was removed in vacuo and the residue treated with distilled water. The aqueous solution was extracted with DCM (3 times). The organics were combined dried with anhydrous sulphate, filtered and evaporated to give the title compound as a brown solid (1.12 g, 69%). NMR (CDCl₃): 2.75 (s, 3H), 4.65 (s, 2H), 8.13 (s, 1H), 8.2 (s, 1H); m/z 142.

### Method 5

### 4-(1-Iso-propyl-2-methyl-1H-imidazol-5-yl)pyrimidin-2-ol acetate salt

2-Amino-4-(1-isopropyl-2-methyl-1H-imidazol-5-yl)pyrimidine (Method 39 of WO 03/076436; 1.0 g, 4.6 mmol) was stirred and heated at 60° C. in 70% acetic acid-water (29 ml) and a solution of sodium nitrite (1.1 g, 16 mmol) in water (2 ml) was added over 2 minutes. The mixture was stirred and heated at 60° C. for 3 hours. The reaction was allowed to cool and the pH of the solution adjusted to 7.0 using 40% sodium hydroxide. More water (10 ml) was added and the mixture was extracted with EtOAc (3 times). The organics were combined, dried with anhydrous sodium sulphate, filtered and evaporated to give the title compound as a white solid (0.94 g, 73%). NMR (CDCl₃): 1.6 (d, 6H), 2.1 (s, 3H), 2.63 (s, 3H), 6.0 (m, 1H), 6.68 (d, 1H), 7.63 (s, 1H), 7.68 (d, 1H); m/z 218.

### Method 6

### 2-Chloro-4-(1-isopropyl-2-methyl-1H-imidazol-5-yl)pyrimidine

4-(1-Isopropyl-2-methyl-1H-imidazol-5-yl)pyrimidin-2-ol acetate salt (Method 5; 821 mg, 2.95 mmol) was stirred and heated at reflux in a mixture of phosphorous oxychloride (12.6 ml) and phosphorous pentachloride (0.69 g) under nitrogen for 24 hours. Excess phosphorous oxychloride was removed in vacuo. The residue was dissolved in DCM and the solution stirred in ice and water added. The mixture was treated with 40% sodium hydroxide to pH 11. The layers were partitioned and separated and the organics washed with saturated sodium chloride, dried with anhydrous sodium sulphate, filtered and evaporated. The crude product was purified by flash chromatography eluting with MeOH:DCM (2:98) to give the title compound as a brown gum (512 mg, 73.5%). NMR: 1.5 (d, 6H), 2.5 (s, 3H), 5.23 (m, 1H), 7.7 (s, 1H), 7.77 (d, 1H), 8.6 (d, 1H); m/z 237.

### Method 7

### 5-(Methylthio)pyridin-2-amine hydrochloride salt

To a mixture of 5-iodopyridin-2-amine (2.0 g, 9.09 mmol), anhydrous potassium carbonate (2.5 g, 18.18 mmol), sodium methanethiolate (1.27 g, 18.18 mmol) and cuprous iodide (172 mg, 0.909 mmol) in isopropanol (27 ml), ethylene glycol (1.01 ml, 18.18 mmol) was added. The reaction was stirred at 80° C. under nitrogen for 24 hours. The reaction mixture was diluted with EtOAc and washed in turn with water (twice), saturated sodium chloride, dried with anhydrous sodium sulphate, filtered and evaporated. The residue was dissolved in ether and filtered to remove an insoluble impurity. The filtrate was taken and treated with excess hydrogen chloride in 1,4-dioxane. The precipitated solid was filtered, washed with ether and dried to give the title compound as a white solid (1.385 g, 86%). NMR: 2.43 (s, 3H), 7.0 (d, 1H), 7.9 (m, 2H); m/z 140.

### Method 8

### di-tert-Butyl ((Z)-{[5-(methylthio)pyridin-2-yl]amino}methylylidene)biscarbamate

To a solution of tert-butyl N-[N-[(2-methylpropan-2-yl)oxycarbonyl]-N′-(trifluoromethylsulfonyl)carbamimidoyl]carbamate (221 mg, 0.566 mmol) and triethylamine (158 μl, 1.132 mmol) in dry DCM (3.0 ml), 5-(methylthio)pyridin-2-amine hydrochloride salt (Method 7; 100 mg, 0.566 mmol) was added. The solution was stirred at room temperature under nitrogen for 120 hours. The reaction mixture was diluted with additional DCM and washed with water (twice), saturated sodium bicarbonate solution (twice), water and saturated sodium chloride. The solution was dried with anhydrous sodium sulphate, filtered and evaporated. The crude product was purified by chromatography eluting with EtOAc:isohexane (5:95) to give the title compound as a gum (95 mg, 44%). NMR: 1.47 (s, 18H), 2.53 (s, 3H+ DMSO), 7.83 (d, 1H), 8.15 (d, 1H), 8.23 (d, 1H), 10.54 (s, 1H), 11.35 (s, 1H); m/z 383.

### Method 9

### N-[5-(Methylthio)pyridin-2-yl]guanidine

A solution di-tert-butyl ((Z)-{[5-(methylthio)pyridin-2-yl]amino}methylylidene) biscarbamate (Method 8; 1.57 g, 4.1 mmol) in trifluoroacetic acid (28 ml) and water (3.1 ml) was stirred at room temperature overnight. The water and excess trifluoroacetic acid were removed in vacuo and the residue azeotroped with toluene (twice). The crude salt was dissolved in MeOH (70 ml) and macroporous polystyrene carbonate resin (4 g of capacity 3.0 m.equ per g) was added. The mixture was gently stirred at room temperature for 4 hours. Distilled water (20 ml) was added and the resin filtered of and washed with MeOH-water. The filtrate was evaporated and the residue was azeotroped with toluene to give the title compound as a white solid (618 mg, 93%). NMR: 2.4 (s, 3H), 6.55 (d, 1H), 7.45 (dd, 1H), 8.02 (d, 1H); m/z 182.

### Method 10

### 6-Methyl-5-(methylthio)pyridin-2-amine

To a mixture of 5-iodo-6-methylpyridin-2-amine (prepared as in WO 02/37927; 3.0 g, 12.82 mmol), anhydrous potassium carbonate (3.54 g, 25.64 mmol), sodium methanethiolate (1.8 g, 25.64 mmol) and cuprous iodide (245 mg, 1.28 mmol) in isopropanol (41 ml), ethylene glycol (1.43 ml, 25.64 mmol) was added. The reaction was stirred at 80° C. under nitrogen for 24 hours. The reaction mixture was diluted with EtOAc and filtered. The filter washed with EtOAc. The filtrate was taken and washed with water. The mixture was filtered through a celite pad and the filter was washed with water and EtOAc. The organic layer was separated and washed in turn with water, saturated sodium chloride, dried with anhydrous sodium sulphate, filtered and evaporated. The residue was dissolved in ether and treated with excess hydrogen chloride in 1,4-dioxane. The precipitated solid was filtered, washed with ether and dried. The hydrochloride salt was dissolved in water and the pH of the solution was adjusted to 12 with 40% sodium hydroxide solution. The aqueous layer was extracted with DCM (twice). The organic layers were combined, dried with anhydrous sodium sulphate, filtered and evaporated to give the title compound as a waxy solid (1.78 g, 90%). NMR: 2.25 (s, 3H), 2.34 (s, 3H), 5.87 (s, 2H), 6.27 (d, 1H), 7.33 (d, 1H); m/z 155.

### Method 11

### di-tert-Butyl ((Z)-{[6-methyl-5-(methylthio)pyridin-2-yl]amino}methylylidene)biscarbamate

To a solution of tert-butyl N-[N-[(2-methylpropan-2-yl)oxycarbonyl]-N′-(trifluoromethylsulfonyl)carbamimidoyl]carbamate (4.1 g, 10.51 mmol) and triethylamine (1.46 ml, 10.51 mmol) in dry DCM (51 ml), 6-methyl-5-(methylthio)pyridin-2-amine (Method 10; 1.62 g, 10.51 mmol) was added. The solution was stirred at reflux under nitrogen for 96 hours. N,N-Dimethylethylenediamine (1.15 ml, 10.51 mmol) was added and the solution stirred at room temperature for 2½ hours. The reaction mixture was concentrated in vacuo and the residue dissolved in ether and water and the layers partitioned and separated. The organic layer was washed in turn with water, 0.25M citric acid (twice), water, dilute sodium bicarbonate solution, water and saturated sodium chloride and dried over anhydrous sodium sulphate, filtered and evaporated to give the title compound as a pink solid (1.32 g, 31%). NMR: 1.55 (s, 18H), 2.43 (s, 3H), 2.5 (s, 3H), 7.5 (d, 1H), 8.2 (s, 1H), 10.7 (s, 1H), 11.52 (s, 1H); m/z 397.

### Method 12

### N-[6-Methyl-5-(methylthio)pyridin-2-yl]guanidine

A solution of di-tert-butyl ((Z)-{[6-methyl-5-(methylthio)pyridin-2-yl]amino}methylylidene)biscarbamate (Method 11; 673 mg, 1.7 mmol) in trifluoroacetic acid (12 ml) and water (1.25 ml) was stirred at room temperature overnight. The water and excess trifluoroacetic acid were removed in vacuo and the residue azeotroped with toluene (twice). The residue was suspended and stirred in distilled water (20 ml) and the pH of the suspension adjusted to 13 using 40% sodium hydroxide solution (pH meter). The solution was extracted with EtOAc (4 times) and the organics combined, dried with anhydrous sodium sulphate, filtered and evaporated. The crude product was triturated with ether, filtered washed with ether and dried to give the title compound as a white solid (237 mg, 71%). NMR: 2.33 (s, 3H), 2.4 (s, 3H), 6.56 (d, 1H), 7.47 (d, 1H); m/z 197.

### Method 13

### N-(6-Morpholin-4-ylpyridin-3-yl)guanidine hydrochloride salt

6-Morpholino-3-pyridinamine (1 g, 5.58 mmol, 1 eq) and cyanamide (293 mg, 6.98 mmol, 1.25 eq) were combined in 1,4-dioxane (15 ml) at room temperature. A 4.0M HCl solution in 1,4-dioxane (2.1 ml, 8.37 mmol, 1.5 eq) was added and the mixture heated to 95° C. for 24 hours. After this time the mixture was concentrated and washed thoroughly with ether, yielding the crude HCl salt as a brown solid (1.52 g, 94%). NMR: 3.53 (t, 4H), 3.70 (t, 4H), 7.03 (d, 1H), 7.48 (bs, 3H), 7.56 (dd, 1H), 8.00 (d, 1H) and 9.70 (s, 1H).

### Method 14

### 4-(3-Chloro-5-nitropyridin-2-yl)morpholine

Morpholine (4 ml, excess) and 2,3-dichloro-5-nitropyridine (1.5 g, 7.77 mmol, 1 eq) were combined at 0° C. then allowed to warm to room temp and stirred for 24 hours. After this time the mixture was poured into cold water (100 ml) then filtered. The solid was thoroughly washed with water, and ether before drying to give the product (1.11 g, 60%), as a yellow solid. NMR: 3.73 (t, 4H), 3.84 (t, 4H), 8.33 (m, 1H), 8.97 (m, 1H); m/z 244.

### Method 15

### 5-Chloro-6-morpholin-4-ylpyridin-3-amine

Hydrogen gas was introduced to a degassed suspension of 4-(3-chloro-5-nitropyridin-2-yl)morpholine (Method 14; 1.01 g, 4.15 mmol, 1 eq) and Pd/C 10% catalyst (100 mg, catalytic) in EtOH (50 ml). The reaction was stirred for 24 hrs at room temp, the mixture was filtered and concentrated, yielding the desired product as a pale brown solid (884 mg, 100%). M/z 214.

### Method 16

### N-(5-Chloro-6-morpholin-4-ylpyridin-3-yl)guanidine bicarbonate salt

5-Chloro-6-morpholin-4-ylpyridin-3-amine (Method 15; 800 mg, 3.76 mmol, 1 eq) and cyanamide (205 mg, 4.88 mmol, 1.3 eq) were combined in 1,4-dioxane (25 ml) and 4.0M HCl solution in 1,4-dioxane (1.22 ml, 4.88 mmol, 1.3 eq) was added. The mixture was heated to 95° C. and stirred at this temperature overnight. After this time the mixture was concentrated and redissolved in water (10 ml). A saturated NaHCO₃ (15 ml) solution was slowly added and the mixture was stirred for approx 18 hrs, after which time the precipitate was filtered off, washed with cold water (3×10 ml) and thoroughly dried to give the product (900 mg, 76%) as a beige solid. M/z 256.

### Method 17

Ethyl 6-{[4-(1-isopropyl-2-methyl-1H-imidazol-5-yl)pyrimidin-2-yl]amino}nicotinate 2-Amino-4-(1-isopropyl-2-methyl-1H-imidazol-5-yl)pyrimidine (Method 39 of WO 03/076436, 477.4 mg, 2.2 mmol), ethyl 6-chloronicotinate (373 mg, 2 mmol), tris(dibenzylideneacetone)dipalladium(0) (9.2 mg, 0.5 mol %), BINAP (12.5 mg, 1 mol %) and caesium carbonate (912.3 mg, 2.8 mmol) in anhydrous 1,4-dioxane (6 ml) were evacuated and refilled with nitrogen (3 times). The reaction was heated under nitrogen at 100° C. overnight. The residue obtained after evaporation of solvent under reduced pressure was partitioned between DCM and water and the aqueous layer was extracted with DCM twice. The organics were combined, washed with brine, dried and the solvent was evaporated to give a solid which was purified by chromatography eluting with MeOH:DCM:EtOAc (1:49.5:49.5 to 10:45:45). After trituration with ether and evaporation of the solvent, the title compound was obtained as a solid (1:2 mixture ethyl:methyl ester) which was dried in vac oven overnight at 50° C. (293.5 mg, 40%). NMR for ethyl compound (400 MHz): 1.34 (t, 3H), 1.48 (d, 6H), 2.58 (s, 3H under DMSO signal), 4.33 (q, 2H), 5.93 (septet, 1H), 7.29 (d, 1H), 7.57 (s, 1H), 8.23 (dd, 1H), 8.31 (d, 1H), 8.52 (d, 1H), 8.84 (d, 1H), 10.43 (s, 1H); m/z 367 & m/z 353.

### Method 18

### 6-{[4-(1-Isopropyl-2-methyl-1H-imidazol-5-yl)pyrimidin-2-yl]amino}nicotinic acid

Ethyl 6-{[4-(1-isopropyl-2-methyl-1H-imidazol-5-yl)pyrimidin-2-yl]amino}nicotinate (Method 17; 193.4 mg, 0.54 mmol) and 2.5N aq NaOH (0.1 ml) in THF/H₂O (1.5 ml/1 ml) were heated under reflux for 3 h. The residue obtained on evaporation of solvent under reduced pressure was dissolved in MeOH, inorganic material filtered off and re-evaporation of solvent to give the title compound as a solid (133.7 mg, 73%). M/z 339.

### Methods 19-21

The following compounds were prepared by the procedure of Method 18 from the starting materials given.

| Meth | Compound | M/z | SM |
|---|---|---|---|
| 19 | 6-{[5-Fluoro-4-(1-isopropyl-2-methyl-1H- | 357 | Method 22 |
| | imidazol-5-yl)pyrimidin-2-yl]amino}nicotinic | | |
| | acid | | |
| 20 | 6-{[5-Fluoro-4-(1-isopropyl-2-methyl-1H- | 357 | Method 23 |
| | imidazol-5-yl)pyrimidin-2-yl]amino}pyrid- | | |
| | azine-3-carboxylic acid | | |
| 21 | 6-{[4-(1-Isopropyl-2-methyl-1H-imidazol-5- | 353 | Method 25 |
| | yl)pyrimidin-2-yl]amino}-2-methylnicotinic | | |
| | acid | | |

### Method 22

### Methyl 6-{[5-fluoro-4-(1-isopropyl-2-methyl-1H-imidazol-5-yl)pyrimidin-2-yl]amino}nicotinate

The title compound was prepared from 2-amino-5-fluoro-4-(1-isopropyl-2-methyl-1H-imidazol-5-yl)pyrimidine (Method 1; 775.5 mg, 3.3 mmol) and methyl 6-chloronicotinate (514.7 mg, 3 mmol) by the procedure of Method 17. The reaction was heated under nitrogen at 100° C. for 3 h. Extra tris(dibenzylideneacetone)dipalladium(0) (7 mg, 0.25 mol %) and BINAP (9.35 mg, 0.5 mol %) were added and the reaction mixture was heated under nitrogen at 100° C. for 5.5 h before evaporating under reduced pressure. The residue obtained was partitioned between DCM and water and the aqueous layer was extracted with DCM twice. The precipitate formed was filtered off to give pure product. The organics were combined, washed with brine, dried and concentrated. Chromatography eluting with MeOH:DCM:EtOAc (1:49.5:49.5 to 10:45:45) gave a solid which required further purification by reverse phase chromatography (acidic prep HPLC system). The product containing fractions were passed through a pre-equilibrated Isolute SCX-2 column, eluted with MeOH, and a 7 molar solution of ammonia in MeOH. Evaporation of solvent gave the title compound as a solid which was dried in vac oven overnight at 50° C. (735.9 mg, 66%). NMR (400 MHz): 1.48 (d, 6H), 2.55 (s, 3H), 3.86 (s, 3H), 5.71 (septet, 1H), 7.49 (d, 1H), 8.17 (d, 1H), 8.23 (dd, 1H), 8.68 (d, 1H), 8.83 (d, 1H), 10.58 (s, 1H); ¹⁷F NMR (400 MHz): −144.55 (t, 1F); m/z 371.

### Method 23

### Ethyl 5-{[5-fluoro-4-(1-iso-propyl-2-methyl-1H-imidazol-5-yl)pyrimidin-2-yl]amino}pyridine-2-carboxylate

2-Amino-5-fluoro-4-(1-isopropyl-2-methyl-1H-imidazol-5-yl)pyrimidine (Method 1; 517 mg, 2.2 mmol), ethyl 5-bromopyridine-2-carboxylate (460.12 mg, 2 mmol), tris(dibenzylideneacetone)dipalladium(0) (18.31 mg, 1 mol %), Xantphos (25.5 mg, 2.2 mol %) and caesium carbonate (912.3 mg, 2.8 mmol) in anhydrous 1,4-dioxane (8 ml) were evacuated and refilled with nitrogen (3 times). The reaction was heated under nitrogen at 100° C. for 3.5 h. Extra tris(dibenzylideneacetone)dipalladium(0) (18.31 mg, 1 mol %) and Xantphos (25.5 mg, 2.2 mol %) were added and the reaction mixture was heated under nitrogen at 100° C. overnight before evaporating under reduced pressure. The residue obtained was partitioned between DCM and water and the aqueous layer was extracted with DCM twice. The organics were combined, washed with brine, dried and the solvent was evaporated to give a foam which was purified by reverse phase chromatography (acidic prep HPLC system). The product containing fractions were passed through a pre-equilibrated Isolute SCX-2 column, eluted with MeOH, and a 7 molar solution of ammonia in MeOH. Evaporation of solvent gave the title compound as a solid which was dried in vac oven overnight at 50° C. (540 mg, 70%). NMR (400 MHz): 1.33 (t, 3H), 1.48 (d, 6H), 2.54 (s, 3H), 4.32 (q, 2H), 5.39 (septet, 1H), 7.41 (d, 1H), 8.02 (d, 1H), 8.35 (dd, 1H), 8.67 (d, 1H), 8.91 (d, 1H), 10.16 (s, 1H); ¹⁷F NMR (400 MHz): −145.98 (t, 1F); m/z 385.

### Method 24

### 5-{[5-Fluoro-4-(1-isopropyl-2-methyl-1H-imidazol-5-yl)pyrimidin-2-yl]amino}pyridine-2-carbonitrile

The title compound was prepared from 2-amino-5-fluoro-4-(1-isopropyl-2-methyl-1H-imidazol-5-yl)pyrimidine (Method 1; 517 mg, 2.2 mmol) and 5-bromopyridine-2-carbonitrile (366 mg, 2 mmol) by the procedure of Method 23. The reaction was heated under nitrogen at 100° C. overnight before evaporating under reduced pressure. The residue obtained was partitioned between DCM and water and the aqueous layer was extracted with DCM twice. The precipitate formed was filtered off to give pure product. The organics were combined, washed with brine, dried and the solvent was evaporated to give a solid which was purified by reverse phase chromatography (acidic prep HPLC system). The product containing fractions were passed through a pre-equilibrated Isolute SCX-2 column, eluted with MeOH, and then a 7 molar solution of ammonia in MeOH. Evaporation of solvent gave the title compound as a solid (combined with solid collected from aqueous work up) which was dried in vac oven overnight at 50° C. (351 mg, 52%). NMR (400 MHz): 1.48 (d, 6H), 2.55 (s, 3H), 5.36 (septet, 1H), 7.42 (d, 1H), 7.95 (d, 1H), 8.69 (d, 1H), 8.98 (d, 1H), 10.31 (s, 1H); ¹⁷F NMR (400 MHz): −145.24 (t, 1F); m/z 338.

### Method 25

### Ethyl 6-{[4-(1-isopropyl-2-methyl-1H-imidazol-5-yl)pyrimidin-2-yl]amino}-2-methylnicotinate

The title compound was prepared from 2-amino-4-(1-isopropyl-2-methyl-1H-imidazol-5-yl)pyrimidine (Method 39 of WO 03/076436, 501.3 mg, 2.31 mmol) and ethyl 6-chloro-2-methylnicotinate (Method 3; 419 mg, 2.1 mmol) by the procedure of Method 17 (1 mol % Pd and 1.5 mol % BINAP). The residue obtained on evaporation of solvent was partitioned between DCM and water and the aqueous layer was extracted with DCM twice. The organics were combined, washed with brine, dried and concentrated. Purification by reverse phase chromatography (acidic prep HPLC system). The product containing fractions were passed through a pre-equilibrated Isolute SCX-2 column, eluting with MeOH, and then a 7 molar solution of ammonia in MeOH. Evaporation of solvent gave the title compound as a solid that was dried in vac oven overnight at 50° C. (395.4 mg, 50%). NMR (400 MHz): 1.33 (t, 3H), 1.48 (d, 6H), 2.53 (s, 3H under DMSO signal), 2.70 (s, 3H), 4.30 (q, 2H), 5.92 (septet, 1H), 7.27 (d, 1H), 7.56 (s, 1H), 8.18 (s, 2H), 8.50 (d, 1H), 10.20 (s, 1H); m/z 381.

### Method 26

### (2Z)-3-(Dimethylamino)-2-fluoro-1-(1-isopropyl-2-methyl-1H-imidazol-5-yl)prop-2-2-en-1-one

To a stirred solution of (2E)-3-(dimethylamino)-1-(1-isopropyl-2-methyl-1H-imidazol-5-yl)prop-2-en-1-one, (Method 24 of WO 03/076436; 5.53 g, 25 mmol) in MeOH (100 ml) at ambient temperature, was added in portions over 5 mins, Selectfluor (14.16 g, 40 mmol). The temperature was maintained at 25-30° C. by slight cooling. After stirring for 90 min the reaction mixture was cooled in ice/acetone and filtered. The filtrate was evaporated under reduced pressure and the residue was taken into DCM. It was washed with aq. ammonia, brine, dried over anhydrous Na₂SO₄ and evaporated under reduced pressure. The title compound was isolated by MPLC using two separate columns (10% EtOH:EtOAc, then 3.5% EtOH:DCM) as a golden viscose oil, which crystallized on standing over several weeks. Yield=2.50 g (42%). NMR: 1.40 (d, 6H), 2.38 (s, 3H), 3.05 (s, 6H), 4.70 (septet, 1H), 6.96 (d, 1H), 7.08 (s, 1H); ¹⁷F NMR (376 MHz): −166.7 (d); m/z 240.

### Method 27

### Methyl 5-[4-(2-methyl-3-propan-2-yl-imidazol-4-yl)pyrimidin-2-yl]aminopyridine-2-carboxylate

2-Amino-4-(1-isopropyl-2-methyl-1H-imidazol-5-yl)pyrimidine (Method 39 of WO 03/076436) (5.0 g, 23 mmol) was stirred in dioxane (150 ml) and methyl-5-bromopyridine-2-carboxylate (4.73 g, 21.9 mmol) was added. The reaction was purged with nitrogen for 20 mins then palladium (II) acetate (0.295 g, 1.31 mmol), XANTPHOS (1.14 g, 1.97 mmol) and cesium carbonate (14.27 g, 43.8 mmol) were added and the mixture heated at reflux for 3 hours. The mixture was cooled to ambient temperature and the insoluble solids removed by filtration. The solvent was evaporated to give a brown gum (12.57 g). This was dissolved in DCM and purified by flash chromatography using 5% MeOH/DCM as eluent. Solvent evaporation gave the product as a colourless foam (4.02 g, 52%); ¹H NMR (400.132 MHz) 1.49 (d, 6H), 2.50 (s, 3H), 3.86 (s, 3H), 5.64 (m, 1H), 7.21 (d, 1H), 7.49 (s, 1H), 8.03 (d, 1H), 8.41 (d, 1H), 8.51 (d, 1H), 8.95 (s, 1H), 10.06 (s, 1H); m/z 353.

### Method 28

### 5-[4-(2-Methyl-3-propan-2-yl-imidazol-4-yl)pyrimidin-2-yl]aminopyridine-2-carboxylate lithium salt

Methyl 5-[4-(2-methyl-3-propan-2-yl-imidazol-4-yl)pyrimidin-2-yl]aminopyridine-2-carboxylate (Method 27; 4.02 g, 11.4 mmol) was dissolved in EtOH (100 ml) and a solution of lithium hydroxide (0.273 g, 11.4 mmol) in water (25 ml) was added. The yellow solution was stirred at ambient temperature for 19 hours. The solvent was evaporated and the residue dissolved in water (200 ml) and extracted with EtOAc (2×200 ml). The aqueous solution was evaporated to a white solid which was dried under in vacuo at 40° C. to give the title compound (3.93 g, 100%); ¹H NMR (400.132 MHz) 1.45 (d, 6H), 5.62 (m, 1H), 7.13 (d, 1H), 7.45 (s, 1H), 7.94 (d, 1H), 8.26 (d, 1H), 8.45 (d, 1H), 8.72 (s, 1H), 9.81 (s, 1H); m/z 339.

### Method 29

### 5-Bromopyridine-2-carboxylate lithium salt

Methyl-5-bromopyridine-2-carboxylate (5.02 g, 23.24 mmol) was suspended in EtOH (200 mL) then a solution of lithium hydroxide (557 mg, 23.24 mmol) in water (45 mL) was added over 5 mins. The reaction mixture was stirred at ambient temperature for 2 hrs then evaporated to give a yellow paste and the residue partitioned between water (1.2 L) and EtOAc (600 mL). The aqueous layer was separated, washed with additional EtOAc (600 mL) and evaporated to dryness to give a solid which was dried in vacuo to give the title compound (4.9 g); ¹H NMR (400.132 MHz) 7.90 (d, 1H), 8.09 (dd, 1H), 8.57 (d, 1H); m/z 202.

### Method 30

### 5-Bromo-N-(1-methyl-4-piperidyl)-pyridine-2-carboxamide

5-Bromopyridine-2-carboxylate lithium salt (Method 29; 1 g, 4.81 mmol) was suspended in DMF (40 ml), HBTU (2.01 g, 5.29 mmol) was added and the reaction mixture allowed to stir at ambient temperature for 40 mins. DIPEA (2 ml, 11.47 mmol) was added then 4-amino-1-methylpiperidine (632 mg, 5.53 mmol). The reaction was stirred at ambient temperature under an inert atmosphere for 62 hrs then concentrated in vacuo. The residue was partitioned between 2M NaOH (50 ml) and DCM (50 ml). The aqueous layer was extracted with DCM (2×75 ml) and the combined organics washed with brine, dried and evaporated to give a brown gum. Purification by flash chromatography on silica, eluting with 10% MeOH in DCM gave a gum. Ether was added and the mixture was re-evaporated to give the title product as a beige solid (1.05 g); ¹H NMR (400.132 MHz) 1.69 (m, 4H), 2.02 (m, 2H), 2.19 (s, 3H), 2.76 (m, 2H), 3.76 (m, 1H), 7.96 (m, 1H), 8.25 (m, 1H), 8.54 (d, 1H), 8.76 (m, 1H); m/z 298.

### Methods 31-33

The following compounds were prepared by the procedure of Method 30 from the starting materials given.

| Meth | Compound | NMR | M/z | SM |
|---|---|---|---|---|
| 31 | (5-Bromopyridin-2- | ¹H NMR (400.132 MHz) | 286 | Method |
| | yl)-(4-methylpiper- | 2.20 (s, 3H), 2.27 (m, 2H), | | 29 and |
| | azin-1-yl)-metha- | 2.38 (m, 2H), 3.37 (m, 2H), | | 1- |
| | none | 3.63 (m, 2H), 7.55 (m, 1H), | | methyl- |
| | | 8.18 (m, 1H), 8.73 (m, 1H) | | |
| 32 | (5-Bromopyridin-2- | ¹H NMR (400.132 MHz) | 300 | Method |
| | yl)-(4-methyl-1,4- | 1.77 (m, 1H), 1.89 (m, 1H), | | 29 and |
| | diazepan-1-yl)- | 2.29-2.35 (m, 3H), 2.60 (m, | | 1- |
| | methanone | 3H), 2.72 (m, 1H), 3.38- | | methyl- |
| | | 3.46 (m, 2H), 3.65 (m, 2H), | | homo |
| | | 7.54 (m, 1H), 8.18 (m, 1H), | | piper- |
| | | 8.72 (m, 1H) | | azine |
| 33 | (5-Bromopyridin-2- | ¹H NMR (400.132 MHz) | 300 | Method |
| | yl)-(3S- | 1.72 (m, 1H), 2.05 (m, 1H), | | 29 and |
| | dimethylamino- | 2.12 (s, 3H), 2.19 (s, 3H), | | (3S)- |
| | pyrrolidin-1-yl)- | 2.69 (m, 1H), 3.23 (m, | | (−)-3- |
| | methanone | 0.5H), 3.41 (m, 1H), 3.58- | | (Di- |
| | | 3.82 (m, 2.5H), 7.70 (m, | | methyl- |
| | | 1H), 8.19 (m, 1H), 8.74 (m, | | amino)- |
| | | 1H) | | Pyrro- |
| | | | | lidine |

### Method 34

### Cyclopropanecarboxylic acid {1-[1-amino-meth-(Z)-ylidene]-2-oxo-propyl}-isopropyl-amide

Cyclopropanecarboxylic acid isopropyl-(5-methyl-isoxazol-4-yl)-amide (Method 36 in WO03/076434; 18 g, 0.086 mol) and 10% palladium on carbon (3.0 g) in EtOH were reacted with hydrogen at 4 atm of pressure. The reaction was filtered and solvent removed in vacuo to yield a solid, ether was added and the solid was filtered (7.9 g, 44%); 1-m/z 211.

### Method 35

### 1-(2-Cyclopropyl-3-iso-propyl-3H-imidazol-4-yl)-ethanone

Cyclopropanecarboxylic acid {1-[1-amino-meth-(Z)-ylidene]-2-oxo-propyl}-isopropyl-amide (Method 34; 7.9 g, 0.038 mol) and sodium hydroxide (2.28 g, 0.057 mol) were added to EtOH (150 ml) and heated at reflux overnight. The solvent was removed in vacuo and the resulting solid was treated with saturated NH₄Cl (100 ml), extracted with ether (3×100 ml), dried and solvent removed in vacuo to yield a black oil. Purification by column chromatography on silica using 100% ether gave the title compound as a yellow oil (3.9 g, 53%). ¹H NMR (400.132 MHz, CDCl₃) 1.07-1.03 (m, 2H), 1.17-1.11 (m, 2H), 1.57 (d, 6H), 1.98-1.91 (m, 1H), 2.44 (s, 3H), 5.63-5.48 (m, 1H), 7.65 (s, 1H); m/z 193.

### Method 36

### (E)-1-(2-Cyclopropyl-3-isopropyl-3H-imidazol-4-yl)-3-dimethylamino-propenone

1-(2-Cyclopropyl-3-isopropyl-3H-imidazol-4-yl)-ethanone (Method 35; 3.74 g, 0.019 mol) and DMFDMA (6.66 ml, 0.039 mol) were added to DMF and heated at 13° C. for 4 hours. The solvent was removed in vacuo to yield an orange gum, DCM was added followed by ether to give the title compound as a yellow solid which was filtered and dried (4.5 g, 96%). ¹H NMR (400.132 MHz, CDCl₃) 1.03-0.98 (m, 2H), 1.13-1.09 (m, 2H), 1.60 (d, 6H), 1.98-1.92 (m, 1H), 3.12-2.88 (m, 6H), 5.50 (d, 1H), 5.61 (septet, 1H), 7.40 (s, 1H), 7.63 (d, 1H); m/z 248.

### Method 37

### 4-(2-Cyclopropyl-3-isopropyl-3H-imidazol-4-yl)-pyrimidin-2-ylamine

(E)-1-(2-Cyclopropyl-3-isopropyl-3H-imidazol-4-yl)-3-dimethylamino-propenone (Method 36; 4.5 g, 0.019 mol) and guanidine carbonate (6.55 g, 0.036 mol) were added to ethylene glycol ether (75 ml) and heated at 142° C. for 2 days. The solvent was removed in vacuo, water (100 ml) was added then extracted with DCM (3×150 ml), dried and the solvent removed in vacuo to yield a yellow solid. DCM was added followed by ether, the mixture was stirred for 30 minutes before being filtered and dried (3.6 g, 78%); ¹H NMR (400.132 MHz, CDCl₃) 8.22 (d, 1H), 7.28 (s, 1H), 6.79 (d, 1H), 5.57 (septet, 1H), 5.01 (brs, 2H), 2.03-1.96 (m, 1H), 1.64 (d, 6H), 1.17-1.13 (m, 2H), 1.05-1.00 (m, 2H); m/z 244.

1.05-1.00 (m, 2H), 1.17-1.13 (m, 2H), 1.64 (d, 6H), 2.03-1.96 (m, 1H), 5.01 (brs, 2H), 5.57 (septet, 1H), 6.79 (d, 1H), 7.28 (s, 1H), 8.22 (d, 1H); m/z 244.

### Method 38

### N-Isopropyl-acetamidine hydrochloride

Ethyl acetimidate hydrochloride (12.36 g; 0.1 mol) was added to 150 ml pressure tube, followed by ca 60 ml EtOH, forming a white suspension. Isopropylamine (5.91 g; 0.1 mol) was added in a single portion. The tube was sealed, and quickly obtained a clear solution. The solution was heated in oil bath to 95° C. and maintained for 6 h, then another 60 h at ambient temperature. The tube was unsealed and solvent removed under reduced pressure. This material was used without further purification. NMR (400 MHz): 1.12 (d, 6H), 1.98 (s, 3H), 3.00 (s, 6H), 3.55 (m, 1H), 6.98 (br, 2H).

### Method 39

### N-Cyclopentyl-acetaminide hydrochloride

The title compound was prepared by the procedure of Method 38 from cyclopentylamine and ethyl acetimidate hydrochloride.

### Method 40

### 3-Chloro-4,4-diethoxy-butan-2-one

Under nitrogen purge, triethylorthofomate (33.0 ml; 0.2 mol) was added to 500 ml 3-neck round bottom flask and cooled in dry ice/acetone bath to ca −30° C. Boron trifluoride etherate (30.0 ml; 0.24 mol) was placed in an addition funnel, along with ca 100 ml DCM and added dropwise over ca 50 min, maintaining temp around −30° C. throughout (with addition of small amounts of dry ice). After stirring for 15 min at −30° C., the solution was allowed to warm to ambient and then cooled back down to −78° C. in dry ice/acetone bath, causing a thin suspension to form. Chloroacetone (9.2 g; 0.1 mol) was added in a rapid dropwise manner from an addition funnel, and washed in with a small portion of DCM. DIEA (38.8 g; 0.3 mol) was added dropwise over ca 40 min maintaining the temperature <−70° C. throughout. After stirring another hour, the reaction mixture was poured onto stirring sat NaHCO₃. After stirring for ca 15 min, the layers were separated. The aqueous layer was with a small portion of DCM, and then combined organic layer washed with cold dilute (about 1:10 in ice/water) H₂SO₄, and then with water. The organic layer was dried and then stripped to obtain 16.9 g of a dark, emerald green oil. The crude material was distilled (pot temp ca 150° C. under house vacuum) to obtain a light amber oil, wt ca 13 g, about 66% theory). NMR: 1.24 (m, 6H), 2.32 (s, 3H), 3.67 (m, 4H), 4.25 (d, 1H), 4.73 (d, 1H).

### Method 41

### 1-(3-Isopropyl-2-methyl-3H-imidazol-4-yl)-ethanone

Under nitrogen purge, N-isopropyl-acetamidine hydrochloride (Method 38; 3.0 g; 0.02 mol) was stirred with 20 ml CH₃CN. This was followed sequentially by 18-crown-6 (0.26 g; 5 mol %), 3-chloro-4,4-diethoxy-butan-2-one (Method 40; 3.9 g; 0.02 mol), and then potassium carbonate (8.3 g; 0.06 mol). The suspension was heated to reflux, and maintained for 16 h. After cooling, the entire brown suspension was evaporated down under reduced pressure. The resulting residue was partitioned between EtOAc and water. The aqueous phase was extracted twice with EtOAc and the combined organic layer was washed with small portions of water, then saturated sodium chloride solution, and then dried. Removal of solvent under reduced pressure provided 2.1 g of a dark amber oil. Biotage purification (0 to 10% isopropanol in DCM) provided the major material as a honey-coloured, mobile oil (1.6 g; ca 50% theory). NMR: 1.50 (d, 6H), 2.45 (s, 3H), 2.52 (s, 3H), 5.30 (br s, 1H), 7.71 (s, 1H); m/z 167.

### Method 42

### 1-(3-Cyclopentyl-2-methyl-3H-imidazole-4-yl)ethanone

The title compound was prepared by the procedure of Method 41 from N-cyclopentyl-acetaminide hydrochloride (Method 39) and 3-chloro-4,4-diethoxy-butan-2-one (Method 40). Silica gel column purification (5% MeOH/1% TEA/in DCM yielded the product (2.0 g, 68%). NMR (400 MHz): 1.69 (br, 2H), 2.01 (br, 6H), 2.46 (s, 3H), 2.51 (s, 3H), 5.20 (m, 1H), 7.74 (s, 1H); m/z 192.

### Method 43

### 3-Dimethylamino-1-(3-isopropyl-2-methyl-3H-imidazol-4-yl)-propenone

1-(3-Isopropyl-2-methyl-3H-imidazol-4-yl)-ethanone (Method 41; 0.83 g; 0.005 mol) was added to a 15 ml pressure tube, along with ca 5 ml toluene, obtaining a slightly turbid solution. In a single portion, was added DMFDMA (0.66 g; 0.0055 mol) and washed in with a small portion of toluene. The tube was sealed and heated to oil bath temp of 150° C. resulting in a clear, amber solution. After heating for 24 h, the reaction still was ca 50% complete (based upon LC/MS analysis). Another 1.1 equiv of DMF/DMA was added, and the reaction mixture was heated another 16 h, whereupon the reaction was almost complete. Cooling and removal of volatiles under reduced pressure provided a dark oil which went to a semi-solid. This material was extracted few times with hot methylcyclohexane, leaving behind a dark residue. The combined extracts were heated back to reflux and treated with activated charcoal, to obtain a filtrate which deposited solid. This material was heated to obtain a clear, orange solution. Chilling in the refrigerator provided an orange solid. The solid was isolated on the vacuum filter and washed with cold petroleum ether to obtain desired product in about 60% theoretical yield. NMR: 1.54 (d, 6H), 2.55 (s, 3H), 3.01 (br s, 3H), 5.41 (m, 1H), 5.54 (d, 1H), 7.48 (s, 1H), 7.65 (d, 1H); m/z 221.

### Method 44

### 3-Dimethylamino-1-(3-cyclopentyl-2-methyl-3H-imidazol-4-yl)-propenone

The title compound was prepared by the procedure of Method 43 from 1-(3-cyclopentyl-2-methyl-3H-imidazole-4-yl)ethanone (Method 42). Silica gel purification of the crude product column (5% MeOH/1% TEA/in DCM) after evaporation of the solvent yielded the desired product (2.25 g, 87%). NMR (400 MHz): 1.71 (m, 2H), 1.97 (m, 2H), 2.09 (m, 4H), 2.54 (s, 3H), 3.03 (br, 6H), 5.35 (m, 1H), 5.55 (d, 1H), 7.41 (s, 1H), 7.67 (d, 1H); m/z 247.

### Method 45

### N-(5-piperazin-1-yl-pyridin-2-yl)-guanidine hydrochloride

4-(6-((bis-tert-Butoxycarbonyl)guanidino-pyridin-3-yl)-piperazine-1-carboxylic acid tert-butyl ester (Method 53; 2.6 g; 0.005 mol) was added to 250 ml round-bottomed flask, along with ca 75 ml dioxane, providing a pale green suspension. In a single portion, 4N dioxane/HCl (25 ml; 0.1 mol) was added. After ca 6 h, the solvent was removed under reduced pressure, providing a solid. NMR: 3.21 (br s, 4H), 3.39 (m, 4H), 7.02 (d, 1H), 7.62 (dd, 1H), 7.99 (d, 1H), 8.19 (br s, 5H), 9.38 (br s, 2H); m/z 221.

### Method 46

### N-(5-Morpholino-4-yl-pyridin-2-yl)guanidine hydrochloride

The title compound was prepared by the procedure of Method 45 from 4-(6-((bis-tert-butoxycarbonyl)guanidino-pyridin-3-yl)-morpholine (Method 54).

### Method 47

### (2Z)-1-(1-Cyclopentyl-2-methyl-1H-imidazol-5-yl)-3-(dimethylamino)-2-fluoroprop-2-en-1-one

3-Dimethylamino-1-(3-cyclopentyl-2-methyl-3H-imidazol-4-yl)-propenone (Method 44; 2.94 g, 13.30 mmol) in MeOH (80 ml) was cooled into an ice-acetone bath. Selectfluor (5.88 g, 16.7 mmol) was added in one portion. The reaction was stirred at −5° C. for 1 h, and then at room temperature overnight. The solvent was removed, and EtOAc was added to the residue. The resulting solid was purified by silica gel column chromatography (10% MeOH/1% TEA/in DCM) to get the product (1.80 g, 57%). The crude product which purified by silica gel column chromatography (10% MeOH/1% TEA/in DCM) to get the product (2.16 g, 82%). NMR (400 MHz): 1.55 (m, 2H), 1.80 (m, 2H), 1.98 (m, 4H), 2.41 (s, 3H), 3.23 (m, 6H), 4.74 (m, 1H), 7.03 (d, 1H), 7.20 (s, 1H); m/z 265.

### Method 48

### 1-(6-Nitro-pyridin-3-yl)-piperazine

Under a nitrogen purge, the solid 4-bromo-2-nitropyridine (10.1 g; 0.05 mol), potassium carbonate (10.5 g; 0.075 mol; −325 mesh), tetrabutylammonium iodide (1.25 g; 5 mol %), and piperazine (5.4 g; 0.0625 mol) were sequentially added to 80 ml acetonitrile. The suspension was heated to reflux, and maintained for 16 h. The now-bright yellow suspension was filtered hot, and the filter cake washed with a few portions of hot acetonitrile, such that the filtrate flows only slightly yellow. The filtrate quickly deposited a yellow/orange solid. This was reheated to obtain a clear solution, which was placed in the refrigerator for 16 h. The yellow/orange solid was isolated by filtration and the filter cake was washed with small portion cold CH₃CN, followed by a small portion of petroleum ether. Air drying the solid provided ca 10.2 g of solid material, about 65% of theory. Another 2 g of material was isolated by evaporating the acetonitrile filtrate down to a semi-solid and then recrystallizing the residue from a minimum amount of hot isopropanol (treated with activated charcoal). NMR: 1.63 (s, 1H), 2.99 (m, 4H), 3.36 (m, 4H), 7.14 (m, 1H), 8.08 (m, 2H), m/z 209.

### Method 49

### 1-(6-Nitro-pyridin-3-yl)-morpholine

The title compound was prepared by the procedure of Method 48 from 4-bromo-2-nitropyridine and replacing piperazine with morpholine.

### Method 50

### 4-(6-Nitro-pyridin-3-yl)-piperazine-1-carboxylic acid tert-butyl ester

Under a nitrogen purge, 1-(6-nitro-pyridin-3-yl)-piperazine (Method 48; 15.6 g; 0.075 mol) was suspended in ca 120 ml THF. In single portions, triethylamine (10.5 ml; 0.075 mol) and the 4-dimethylaminopyridine (0.46 g; 5 mol %) were added sequentially. Di-t-butyl dicarbonate (16.6 g; 0.075 mol) was dissolved in ca 50 ml THF and placed in an addition funnel. The solution was added dropwise to the stirring suspension, maintaining the temperature below 27° C. throughout by controlling the addition rate. After the addition was complete, the temperature was allowed to drop to ambient temperature, before heating to reflux. After heating for 1 h, a small amount of insolubles were removed by filtration. The solvent was removed under reduced pressure and then the yellow residue was partitioned between EtOAc and water. The aqueous phase was extracted twice with EtOAc. The combined organic layer was washed with small portion of water, then saturated sodium chloride and then dried. Removal of solvent under reduced pressure provide the product. This solid was recrystallized from 2-propanol (treated with activated charcoal) to obtain the product as a crystalline solid, ca 19 g (about 80% theory). NMR: 1.49 (s, 9H), 3.46 (m, 4H), 3.65 (m, 4H), 7.20 (m, 1H), 8.17 (m, 2H). m/z 309.

### Method 51

### 4-(6-Amino-pyridin-3-yl)-piperazine-1-carboxylic acid tert-butyl ester

Under a nitrogen purge, 4-(6-nitro-pyridin-3-yl)-piperazine-1-carboxylic acid tert-butyl ester (Method 50; 15.4 g; 0.05 mol) was suspended in ca 250 ml EtOH. In single portions, cyclohexane (78 ml; 0.75 mol) and then 10% palladium-on-carbon (7.8 g; 7.5 mol %) were added, washing in with some more EtOH. The suspension was heated in an oil bath maintained at 85° C. for 60 h. The reaction mixture was filtered hot through pad of diatomaceous earth. The filter cake was washed with portions of hot EtOH until filtrate came through nearly clear. Volatiles were removed under reduced pressure to obtain 13 g of a beige solid which was further purified by recrystallization from hot methylcyclohexane. NMR: 1.50 (s, 9H), 2.98 (m, 4H), 3.58 (m, 4H), 4.7 (br s, 2H), 6.58 (d, 1H), 7.28 (d, 1H), 7.68 (s, 1H); m/z 279.

### Method 52

### 1-(6-Amino-pyridin-3-yl)-morpholine

The title compound was prepared by the procedure of Method 51 from 4 1-(6-nitro-pyridin-3-yl)-morpholine (Method 49).

### Method 53

### 4-(6-((bis-tert-Butoxycarbonyl)guanidino-pyridin-3-yl)-piperazine-1-carboxylic acid tert-butyl ester

4-(6-Amino-pyridin-3-yl)-piperazine-1-carboxylic acid tert-butyl ester (Method 51; 2.8 g; 0.01 mol) was dissolved in ca 100 ml chloroform. The following were added sequentially, in single portions: triethylamine (3.2 ml; 0.022 mol), bis(t-butoxycarbonyl)thiourea (3.2 g; 0.01 mmol), and 2-chloro-1-methylpyridinium iodide (2.8 g; 0.011 mol). After stinting for 60 h, solvent was removed under reduced pressure and the residue was partitioned between EtOAc and water. The organic layer was washed twice with water, then brine, and then dried. Solvent was removed under reduced pressure to provide a yellow solid. This material was further purified by recrystallization from hot methylcyclohexane. NMR: 1.41 (s, 18H), 1.46 (s, 9H), 3.02 (m, 4H), 3.52 (m, 4H), 7.22 (d, 1H), 7.90 (d, 1H), 8.17 (br d, 1H); m/z 521.

### Method 54

### 4-(6-((bis-tert-Butoxycarbonyl)guanidino-pyridin-3-yl)-morpholine

The title compound was prepared by the procedure of Method 53 from 1-(6-amino-pyridin-3-yl)-morpholine (Method 52).

### Example 53

The following illustrate representative pharmaceutical dosage forms containing the compound of formula (I), or a pharmaceutically acceptable salt or in vivo hydrolysable ester thereof (hereafter compound X), for therapeutic or prophylactic use in humans:—

| (a): Tablet I | mg/tablet |
|---|---|
| Compound X | 100 |
| Lactose Ph. Eur | 182.75 |
| Croscarmellose sodium | 12.0 |
| Maize starch paste (5% w/v paste) | 2.25 |
| Magnesium stearate | 3.0 |
| (b): Tablet II | mg/tablet |
| Compound X | 50 |
| Lactose Ph. Eur | 223.75 |
| Croscarmellose sodium | 6.0 |
| Maize starch | 15.0 |
| Polyvinylpyrrolidone (5% w/v paste) | 2.25 |
| Magnesium stearate | 3.0 |
| (c): Tablet III | mg/tablet |
| Compound X | 1.0 |
| Lactose Ph. Eur | 93.25 |
| Croscarmellose sodium | 4.0 |
| Maize starch paste (5% w/v paste) | 0.75 |
| Magnesium stearate | 1.0 |
| (d): Capsule | mg/capsule |
| Compound X | 10 |
| Lactose Ph. Eur | 488.5 |
| Magnesium stearate | 1.5 |
| (e): Injection I | (50 mg/ml) |
| Compound X | 5.0% w/v |
| 1M Sodium hydroxide solution | 15.0% v/v |
| 0.1M Hydrochloric acid | (to adjust pH to 7.6) |
| Polyethylene glycol 400 | 4.5% w/v |
| Water for injection | to 100% |
| (f): Injection II | 10 mg/ml |
| Compound X | 1.0% w/v |
| Sodium phosphate BP | 3.6% w/v |
| 0.1M Sodium hydroxide solution | 15.0% v/v |
| Water for injection | to 100% |
| (g): Injection III | (1 mg/ml, buffered to pH 6) |
| Compound X | 0.1% w/v |
| Sodium phosphate BP | 2.26% w/v |
| Citric acid | 0.38% w/v |
| Polyethylene glycol 400 | 3.5% w/v |
| Water for injection | to 100% |

Note
The above formulations may be obtained by conventional procedures well known in the pharmaceutical art. The tablets (a)-(c) may be enteric coated by conventional means, for example to provide a coating of cellulose acetate phthalate.

1: A compound of formula (I): 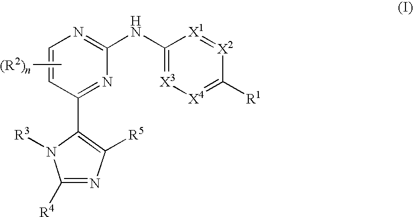
wherein:
R¹ is sulphamoyl, carbamoyl, a group -R⁶-R⁷ or a nitrogen linked 4-7 membered saturated ring which optionally contains an additional nitrogen, oxygen or sulphur atom; wherein said ring may be optionally substituted on carbon by one or more R⁸; and wherein if said ring contains an additional nitrogen atom that nitrogen may be optionally substituted by R⁹;
one of X¹, X², X³ and X⁴ is selected from ═N—, the other three X¹, X², X³ or X⁴ are independently selected from ═N— or ═C(R¹⁰)—;
R² is halo, nitro, cyano, hydroxy, trifluoromethyl, trifluoromethoxy, amino, carboxy, carbamoyl, mercapto, sulphamoyl, C₁₋₃alkyl, C₂₋₃alkenyl, C₂₋₃alkynyl, C₁₋₃alkoxy, C₁₋₃alkanoyl, N—(C₁₋₃alkyl)amino, N,N—(C₁₋₃alkyl)₂amino, C₁₋₃alkanoylamino, N—(C₁₋₃alkyl)carbamoyl, N,N—(C₁₋₃alkyl)₂carbamoyl, C₁₋₃alkylS(O)ₐ wherein a is 0 to 2, N—(C₁₋₃alkyl)sulphamoyl or N,N—(C₁₋₃alkyl)₂sulphamoyl; wherein R² may be optionally substituted on carbon by one or more R¹¹;
n is 0 to 2, wherein the values of R² may be the same or different;
R³ is hydrogen, C₁₋₆alkyl, C₂₋₆alkenyl, C₂₋₆alkynyl, carbocyclyl or heterocyclyl; wherein R³ may be optionally substituted on carbon by one or more R¹²; and wherein if said heterocyclyl contains an —NH— moiety that nitrogen may be optionally substituted by a group selected from R¹³;
R⁴, R⁵ and R⁸ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, C₃₋₈cycloalkyl or a 4-7 membered saturated heterocyclic group; wherein R⁴, R⁵ and R⁸ independently of each other may be optionally substituted on carbon by one or more R¹⁴; and wherein if said 4-7 membered saturated heterocyclic group contains an —NH— moiety that nitrogen may be optionally substituted by a group selected from R¹⁵;
R⁶ is —O—, —C(O)—, —N(R¹⁶)C(O)—, —C(O)N(R¹⁷)—, S(O)ᵣ, —OC(O)N(R¹⁸)SO₂—, —SO₂N(R¹⁹)— or —N(R²⁰)SO₂—; wherein R¹⁶, R¹⁷, R¹⁸, R¹⁹ and R²⁰ are independently hydrogen or C₁₋₆alkyl optionally substituted by one or more R²¹ and r is 0-2;
R⁷ is selected from C₁₋₆alkyl, C₂₋₆alkenyl, C₂₋₆alkynyl, carbocyclyl or heterocyclyl; wherein R⁷ may be optionally substituted on carbon by one or more R²²; and wherein if said heterocyclyl contains an —NH— moiety that nitrogen may be optionally substituted by a group selected from R²³;
R¹⁰ is selected from hydrogen, halo, nitro, cyano, hydroxy, amino, carboxy, carbamoyl, mercapto, C₁₋₆alkyl, C₁₋₆alkoxy, C₂₋₆alkenyl or C₂₋₆alkynyl;
R¹², R²¹ and R²² are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkoxyC₁₋₆alkoxy, C₁₋₆alkoxyC₁₋₆alkoxyC₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₁₋₆alkyl-R²⁴—, heterocyclylC₁₋₆alkyl-R²⁵—, carbocyclyl-R²⁶— or heterocyclyl-R²⁷—; wherein R¹², R²¹ and R²² independently of each other may be optionally substituted on carbon by one or more R²⁸; and wherein if said heterocyclyl contains an —NH— moiety that nitrogen may be optionally substituted by a group selected from R²⁹;
R²⁴, R²⁵, R²⁶ and R²⁷ are independently selected from —O—, —N(R³⁰)—, —C(O)—, —N(R³¹)C(O)—, —C(O)N(R³²)—, —S(O)ₛ—, —SO₂N(R³³)— or —N(R³⁴)SO₂—; wherein R³⁰, R³¹, R³², R³³ and R³⁴ are independently selected from hydrogen or C₁₋₆alkyl and s is 0-2;
R⁹, R¹³, R¹⁵, R²³ and R²⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; wherein R⁹, R¹³, R¹⁵, R²³ and R²⁹ independently of each other may be optionally substituted on carbon by one or more R³⁵; and
R¹¹, R¹⁴, R³⁵ and R²⁸ are independently selected from halo, nitro, cyano, hydroxy, trifluoromethoxy, trifluoromethyl, amino, carboxy, carbamoyl, mercapto, sulphamoyl, methyl, ethyl, cyclopropyl, cyclobutyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt or an in vivo hydrolysable ester thereof.

2: A compound of formula (I), or a pharmaceutically acceptable salt or an in vivo hydrolysable ester thereof, as claimed in claim 1, wherein R¹ is carbamoyl, a group -R⁶-R⁷ or a nitrogen linked 4-7 membered saturated ring which optionally contains an additional nitrogen or oxygen atom; wherein if said ring contains an additional nitrogen atom that nitrogen may be optionally substituted by R⁹; wherein
R⁶ is —C(O)—, —C(O)N(R¹⁷)— or —S(O)ᵣ—; wherein R¹⁷ is hydrogen or C₁₋₆alkyl and r is 0 or 2;
R⁷ is selected from C₁₋₆alkyl, carbocyclyl or heterocyclyl; wherein R⁷ may be optionally substituted on carbon by one or more R²²; and wherein if said heterocyclyl contains an —NH— moiety that nitrogen may be optionally substituted by a group selected from R²³;
R²² is N,N—(C₁₋₆alkyl)₂amino;
R⁹ and R²³ are independently selected from C₁₋₄alkyl or C₁₋₄alkanoyl; wherein R⁹ and R²³ independently of each other may be optionally substituted on carbon by one or more R³⁵; and
R³⁵ is hydroxy.

3: A compound of formula (I), or a pharmaceutically acceptable salt or an in vivo hydrolysable ester thereof, as claimed in claim 1, wherein X⁴ is ═N— and X¹, X² and X³ are independently selected from ═C(R¹⁰)—; or X¹ is ═N— and X³, X² and X⁴ are independently selected from ═C(R¹⁰)—; or X¹ and X⁴ are ═N— and X² and X³ are independently selected from ═C(R¹⁰)—; wherein
R¹⁰ is selected from hydrogen, halo or C₁₋₆alkyl.

4: A compound of formula (I), or a pharmaceutically acceptable salt or an in vivo hydrolysable ester thereof, as claimed in claim 1, wherein R² is halo.

5: A compound of formula (I), or a pharmaceutically acceptable salt or an in vivo hydrolysable ester thereof, as claimed in claim 1, wherein n is 0 or 1.

6: A compound of formula (I), or a pharmaceutically acceptable salt or an in vivo hydrolysable ester thereof, as claimed in claim 1, wherein R³ is C₁₋₆alkyl or carbocyclyl.

7: A compound of formula (I), or a pharmaceutically acceptable salt or an in vivo hydrolysable ester thereof, as claimed in claim 1, wherein R⁴ is methyl or cyclopropyl.

8: A compound of formula (I), or a pharmaceutically acceptable salt or an in vivo hydrolysable ester thereof, as claimed in claim 1, wherein R⁵ is hydrogen.

9: A compound of formula (I), as claimed in claim 1, 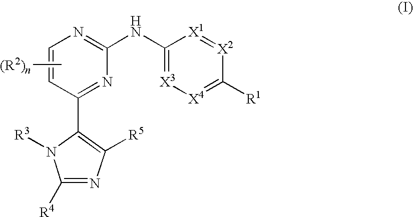
wherein
R¹ is carbamoyl, morpholino, N-methylcarbamoyl, N,N-dimethylcarbamoyl, methylthio, mesyl, N-cyclopropylcarbamoyl, N-ethylcarbamoyl, piperazin-1-yl, 4-((R)-2-hydroxypropionyl)piperazin-1-yl, 4-((S)-2-hydroxypropionyl)piperazin-1-yl, 4-(2-hydroxyacetyl)piperazin-1-yl, 4-(acetyl)piperazin-1-yl, morpholinocarbonyl, 4-methylpiperazin-1-ylcarbonyl, 4-methyl-1,4-diazepanylcarbonyl, N-(1-methylpiperidin-4-yl)carbamoyl and (S)-3-dimethylaminopyrrolidin-1-ylcarbonyl;
X⁴ is ═N— and X¹, X² and X³ are independently selected from ═C(R¹⁰)—; or X¹ is ═N— and X³, X² and X⁴ are independently selected from ═C(R¹⁰)—; or X¹ and X⁴ are ═N— and X² and X³ are independently selected from ═C(R¹⁰)—;
R² is fluoro or chloro;
n is 0 or 1;
R³ is isopropyl or cyclopentyl;
R⁴ is methyl or cyclopropyl;
R⁵ is hydrogen; and
R¹⁰ is selected from hydrogen, chloro or methyl;
or a pharmaceutically acceptable salt or an in vivo hydrolysable ester thereof.

10: A compound of formula (I), 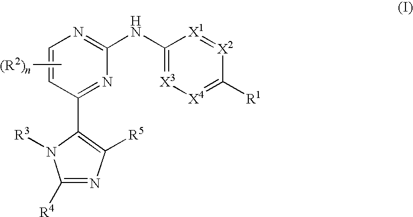
selected from
5-fluoro-4-(1-isopropyl-2-methyl-1H-imidazol-5-yl)-N-(6-morpholin-4-ylpyridin-3-yl)pyrimidin-2-amine;
5-{[5-fluoro-4-(1-isopropyl-2-methyl-1H-imidazol-5-yl)pyrimidin-2-yl]amino}-N-methylpyridine-2-carboxamide;
6-{[5-fluoro-4-(1-isopropyl-2-methyl-1H-imidazol-5-yl)pyrimidin-2-yl]amino}-N-methylnicotinamide;
6-{[5-fluoro-4-(1-isopropyl-2-methyl-1H-imidazol-5-yl)pyrimidin-2-yl]amino}-N,N-dimethylnicotinamide;
5-{[5-fluoro-4-(1-isopropyl-2-methyl-1H-imidazol-5-yl)pyrimidin-2-yl]amino}pyridine-2-carboxamide;
N-cyclopropyl-5-{[5-fluoro-4-(1-isopropyl-2-methyl-1H-imidazol-5-yl)pyrimidin-2-yl]amino}pyridine-2-carboxamide;
N-ethyl-5-{[5-fluoro-4-(1-isopropyl-2-methyl-1H-imidazol-5-yl)pyrimidin-2-yl]amino}pyridine-2-carboxamide;
N-(6-{[(3S)-3-(dimethylamino)pyrrolidin-1-yl]carbonyl}pyridin-3-yl)-4-(1-isopropyl-2-methyl-1H-imidazol-5-yl)pyrimidin-2-amine;
5-chloro-N-(6-{[(3S)-3-(dimethylamino)pyrrolidin-1-yl]carbonyl}pyridin-3-yl)-4-(1-isopropyl-2-methyl-1H-imidazol-5-yl)pyrimidin-2-amine; and
4-(2-cyclopropyl-1-isopropyl-1H-imidazol-5-yl)-N-(6-{[(3S)-3-(dimethylamino)pyrrolidin-1-yl]carbonyl}pyridin-3-yl)pyrimidin-2-amine;
or a pharmaceutically acceptable salt thereof.

11: A process for preparing a compound of formula (I) or a pharmaceutically acceptable salt or an in vivo hydrolysable ester thereof which process, wherein variable groups are, unless otherwise specified, as defined claim 1, comprises of:
Process a) reaction of a pyrimidine of formula (II): 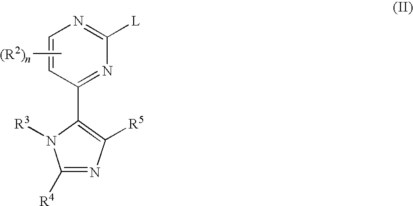
wherein L is a displaceable group; with an aniline of formula (III): 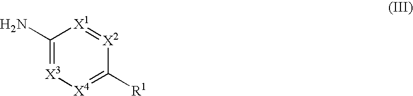
or
Process b) reacting a compound of formula (IV): 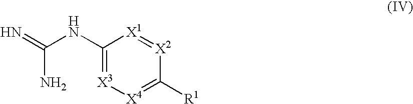
with a compound of formula (V): 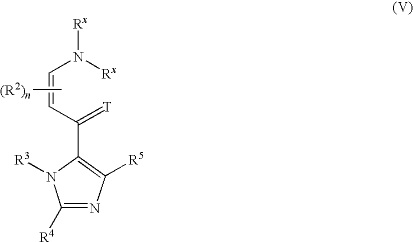
wherein T is O or S; R^{x} may be the same or different and is selected from C₁₋₆alkyl; or
Process c) for compounds of formula (I) wherein R¹ is carbamoyl or —C(O)N(R¹⁷)(R⁷) reacting an acid of formula (VI): 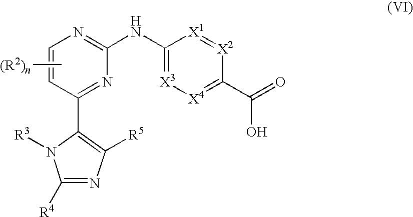
or an activated derivative thereof; with an amine of formula (VII):
HNR⁷′R¹⁷ (VII)
wherein R⁷′ is R⁷ or hydrogen; or
Process d) for compounds of formula (I); reacting a pyrimidine of formula (VIII): 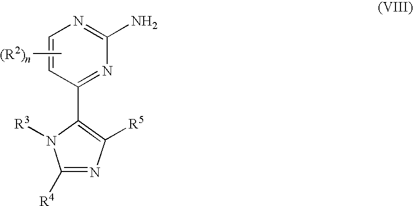
with a compound of formula (IX): 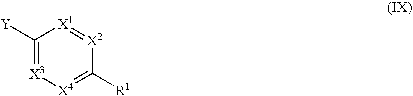
where Y is a displaceable group;
and thereafter optionally:
i) converting a compound of the formula (I) into another compound of the formula (I);
ii) removing any protecting groups; and/or
iii) forming a pharmaceutically acceptable salt or in vivo hydrolysable ester.

12: A pharmaceutical composition which comprises a compound of the formula (I), or a pharmaceutically acceptable salt or in vivo hydrolysable ester thereof, as claimed in claim 1, and a pharmaceutically-acceptable diluent or carrier.

13: A compound of the formula (I), or a pharmaceutically acceptable salt or in vivo hydrolysable ester thereof, as claimed in claim 1, for use as a medicament.

14-18. (canceled)

19: A method of producing an anti-cell-proliferation effect, in a warm-blooded animal in need of such treatment, which comprises administering to said animal an effective amount of a compound of formula (I) or a pharmaceutically acceptable salt or in vivo hydrolysable ester thereof, as claimed in claim 1.

20: A method of producing a CDK2 inhibitory effect, in a warm-blooded animal in need of such treatment, which comprises administering to said animal an effective amount of a compound of formula (I) or a pharmaceutically acceptable salt or in vivo hydrolysable ester thereof, as claimed in claim 1.

21: A method of treating cancer, in a warm-blooded animal in need of such treatment, which comprises administering to said animal an effective amount of a compound of formula (I) or a pharmaceutically acceptable salt or in vivo hydrolysable ester thereof, as claimed in claim 1.

22: A method of treating leukaemia or lymphoid malignancies or cancer of the breast, lung, colon, rectum, stomach, liver, kidney, prostate, bladder, pancreas, vulva, skin or ovary, in a warm-blooded animal in need of such treatment, which comprises administering to said animal an effective amount of a compound of formula (I) or a pharmaceutically acceptable salt or in vivo hydrolysable ester thereof, as claimed in claim 1.

23: A method of treating cancer, fibroproliferative disorders, differentiative disorders, psoriasis, rheumatoid arthritis, Kaposi's sarcoma, haemangioma, acute or chronic nephropathies, atheroma, atherosclerosis, arterial restenosis, autoimmune diseases, acute or chronic inflammation, bone diseases and ocular diseases with retinal vessel proliferation, in a warm-blooded animal in need of such treatment, which comprises administering to said animal an effective amount of a compound of formula (I) or a pharmaceutically acceptable salt or in vivo hydrolysable ester thereof, as claimed in claim 1.

24: The compound of formula (I), or a pharmaceutically acceptable salt or an in vivo hydroylsable ester thereof, as claimed in claim 1, wherein:
R¹ is carbamoyl, a group -R⁶-R⁷ or a nitrogen linked 4-7 membered saturated ring which optionally contains an additional nitrogen or oxygen atom; wherein if said ring contains an additional nitrogen atom that nitrogen may be optionally substituted by R⁹; wherein
R⁶ is —C(O)—, —C(O)N(R¹⁷)— or —S(O)ᵣ—; wherein R¹⁷ is hydrogen or C₁₋₆alkyl and R is 0 or 2;
R⁷ is selected from C₁₋₆alkyl, carbocyclyl or heterocyclyl; wherein R⁷ may be optionally substituted on carbon by one or more R²²; and wherein if said hetrocyclyl contains an —NH— moiety that nitrogen may be optionally substituted by a group selected from R²³;
R²² is N,N—(C₁₋₆alkyl)₂amino;
R⁹ and R²³ are independently selected from C₁₋₄alkyl or C₁₋₄alkanoyl; wherein R⁹ and R²³ independently of each other may be optionally substituted on carbon by one or more R³⁵;
R³⁵ is hydroxy;
R² is halo;
n is 0 or 1;
R³ is C₁₋₆alkyl or carbocyclyl;
R⁴ is methyl or cyclopropyl;
R⁵ is hydrogen; and
X⁴ is ═N— and X¹, X² and X³ are independently selected from ═C(R¹⁰)—; or
X¹ is ═N— and X³, X² and X⁴ are independently selected from ═C(R¹⁰)—; or
X¹ and X⁴ are ═N— and X² and X³ are independently selected from ═C(R¹⁰)—; wherein
R¹⁰ is selected from hydrogen, halo, or C₁₋₆alkyl.
